SEQUENCE LISTING (43 sequences)

SEQ ID NO: 1 - protein, 166 aa, Helicobacter pylori
  SITE 1..166: note = Exemplary H. pylori ferritin
FSKDIEKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 2 - protein, 7 aa, unidentified
  REGION 1..7: note = Exemplary bullfrog ferritin extension sequence
ESQVRSQ

SEQ ID NO: 3 - protein, 46 aa, synthetic construct
  REGION 1..46: note = Synthetic: 46 amino acid linker
GGSGSASSGASASGSSGGSGSGSGSGSSASSGASSGGASGGSGGSG

SEQ ID NO: 4 - protein, 32 aa, synthetic construct
  REGION 1..32: note = Synthetic: 32 amino acid linker
SGGGSGSASSGASASGSSGSGSGSGSSSASSG

SEQ ID NO: 5 - protein, 88 aa, synthetic construct
  REGION 1..88: note = Synthetic: 88 amino acid linker
GGSGSASSGASASGSSGSGSGSGSSSASSGASSGGASGGSGGSGGGSGSASSGASASGSSGSGSGSGSSSASSGASSGGASGGSGGSG

SEQ ID NO: 6 - protein, 44 aa, synthetic construct
  REGION 1..44: note = Synthetic: 44 amino acid linker
GGSGSASSGASASGSSGSGSGSGSSSASSGASSGGASGGSGGSG

SEQ ID NO: 7 - protein, 12 aa, synthetic construct
  REGION 1..12: note = Synthetic: 12 amino acid rigid linker
EPEPEPEPEPGG

SEQ ID NO: 8 - protein, 48 aa, synthetic construct
  REGION 1..48: note = Synthetic: 48 amino acid rigid linker
SGEPEPEPEPEPEPEPEPEPEPEPEPEPEPEPEPEPEPEPEPEPEPEP

SEQ ID NO: 9 - protein, 1299 aa, synthetic construct
  REGION 1..1299: note = Synthetic: C16 gL/gH/gp42/ferritin
NWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAILMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSIAPQEATLDQAAVSQAVDGFLGRLSLEREDRDAWHLPAYKCVDRLDKVLMIIPLINVTFIISSDREVRGSALYEASTTYLSSSLFLSPVILNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWQGCFQACAELYPCTYFYGPTPDILPVVTRSLQAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 10 - protein, 1299 aa, synthetic construct
  REGION 1..1299: note = Synthetic: C17 gL/gH/gp42/ferritin
NWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAILMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSIAPQEATLDQAAVSQAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWQGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 11 - protein, 1299 aa, synthetic construct
  REGION 1..1299: note = Synthetic: C18 gL/gH/gp42/ferritin
NWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 12 - protein, 22 aa, synthetic construct
  REGION 1..22: note = Synthetic: Exemplary mammalian leader sequence
MRAVGVFLAICLVTIFVLPTWG

SEQ ID NO: 13 - protein, 30 aa, synthetic construct
  REGION 1..30: note = Synthetic: Exemplary mammalian leader sequence
MDSKGSSQKGSRLLLLLVVSNLLLPQGVLA

SEQ ID NO: 14 - protein, 600 aa, synthetic construct
  REGION 1..600: note = Synthetic: gp220 ferritin Nanoparticle
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPEGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 15 - protein, 228 aa, synthetic construct
  REGION 1..228: note = Synthetic: Gp42
DSKGSSQKGSRLLLLLVVSNLLLPQGVLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 16 - protein, 115 aa, synthetic construct
  REGION 1..115: note = Synthetic: Exemplary gL polypeptide
NWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 17 - protein, 662 aa, synthetic construct
  REGION 1..662: note = Synthetic: Exemplary gH polypeptide
AASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERA

SEQ ID NO: 18 - protein, 425 aa, synthetic construct
  REGION 1..425: note = Synthetic: Exemplary gp220 polypeptide
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPE

SEQ ID NO: 19 - DNA, 3972 bp, synthetic construct
  misc_feature 1..3972: note = Synthetic: Exemplary polynucleotide encoding a gL/gH/gp42/ferritin nanoparticle (e.g., C16 of SEQ ID NO: 9)
  source 1..3972: mol_type = other DNA
gccaccatgagagctgtgggagtgttcctggccatctgcctggtcaccatcttcgtgctgcctacctggggcaactgggcttacccttgctgtcatgtgacccagctgagagcccagcatctgctggccctggaaaacatctccgacatctacctggtgtccaaccagacctgcgacgccttctctctggcctctctgaactctcctaagcagggctccaaccagctcgtgatctccagatgtgccaacggcctgaacgtggtgtccttcttcatctctatcctgaagcggtccagcagcgccctgaccggacatctgagagagctgctgaccacactggaaaccctgtacggctccttcagcgtggaagatctgtttggcgcccagctgaacagatacgcctggcatagaggtggcggcggatctggctctgcttcttctggtgcttctgcctctggatcctctggcggctctggttccggttctggaagcggatcttctgccagctctggcgcatcttctggcggagctagcggaggctctggtggtagcggagctgcttctctgtccgaagtgaagctgcacctggacatcgagggccacgccagccactatacaatcccttggactgagctgctggccaaggtgccaggactgtctcctgaagctctttggcgcgaggccaacgtgaccgaggatctggcatctatgctgaaccggtacaagctgatctacaagaccagcggcaccctgggaatcgctctggctgagcctgtggatatccctgctgtgtccgagggctctatgcaggtcgacgcttctaaagtgcaccccggcgtgatcagcggcctgaattctcctgcctgtatgctgagcgcccctctggaaaagcagctgttctactacatcggcaccatgctgcccaacaccagacctcactcctacgtgttctaccagctgcggtgccacctgtcttacgtggccctgtctatcaacggcgacaagttccagtacaccggcgctatgacctccaagttcctgatgggcacctacaagagagtgaccgagaagggcgacgagcatgtgctgtctctggtgttcggaaagaccaaggacctgcctgacctgagaggccccttcagctatccctctctgacctccgctcagtccggcgagtattccctggtcatcgtgaccaccttcgtgcactacgccaacttccacaactacttcgtgcccaacctgaaggacatgttctctcgggccgtgacaatgaccgccgcctcttacgctagatacgtgctgcagaaactggtgctgctggaaatgaagggcggctgcagagagcccgagctggaaacagagacactgaccaccatgttcgaggtgtccgtggccttcttcaaagtgggccatgctgtgggcgagacaggcaacggatgtgtggatctgagatggctggctaagagcttcttcgagctgaccgtgctgaaggatatcatcggcatctgctacggcgccaccgtgaagggcatgcagtcttacggactggaaagactggccgccatcctgatggctaccgtgaagatggaagaactgggccacctgaccaccgagaagcaagagtacgctctgagactggccaccgtgggctatcctaaagccggcgtttactccggactgatcggcggagcaacatccgtgctgctgtccgcctacaacagacaccctctgttccagcctctgcacaccgtgatgcgcgagacactgttcatcggctctcacgtggtgctgcgcgagctgagactgaacgtgacaacccagggacctaatctggccctgtatcagctgctgagcaccgctctgtgtagcgctctggaaatcggcgaggtgctgagaggactggctctgggaacagagtccggcctgttctccccttgctacctgtctctgagattcgacctgaccagagataagctgctgtctatcgcccctcaagaggctaccctggatcaggctgcagtgtctcaggccgtggatggctttctgggcagactgtccctggaaagagaggacagggacgcttggcatctgcccgcctataagtgcgtggacagactggacaaggtgctgatgatcattcccctgatcaatgtgaccttcatcatctcctccgaccgggaagtgcgaggctctgctctgtatgaggcctctactacctacctgtcctccagcctgtttctgtcccctgtgatcctgaacaagtgctcccaaggcgctgtggccggcgagcctagacagatccctaagatccagaacttcacccggactcagaagtcctgtatcttctgcggcttcgccctgctgtcctacgacgagaaagaaggcctggaaaccaccacctacatcaccagccaagaggtgcagaactccatcctgtccagcaattacttcgacttcgacaacctgcatgtgcactacctgctgctgactaccaacggcacagtgatggaaatcgccggcctgtacgaggaaagagcatccggcggaggttctggcagtgctagtagtggcgcttccgcttctggctcctccggctctggatctggaagtggctcctctagtgcttccagcggcgctatcacatgggtgcccaagcctaacgtggaagtgtggccagtggatcctcctccacctgtgaacttcaacaagaccgccgagcaagaatacggggacaaagaagtgaaactgccccactggacccctacactgcacacctttcaggtgccccagaactacaccaaggccaactgcacctactgcaatacccgcgagtacacctttagctacaagggctgctgcttctacttcaccaagaagaaacacacctggcagggctgctttcaggcctgtgctgagctgtacccctgtacctacttctacggccccacacctgacatcctgccagtggtcacaagatccctgcaggccatcgagtctctgtgggtcggagtgtacagagtcggcgaaggcaactggacctctctggatggcggaaccttcaaggtgtaccagatcttcggctcccattgcacctacgtgtccaagttctccaccgtgcctgtgtctcaccacgagtgcagctttctgaagccctgcctgtgcgtgtcccagagatctaatagcggcggcagtggctctgcatctagtggcgcaagtgcttccggatcttccggctcaggctccggtagtggatcttctagtgcctcttccggcgcttctagcggtggtgcatctggtggatctggcggaagcggcggaggaagtggatcagcaagttcaggcgcttctgctagcggctctagtggcagtggcagcggttccggatcctcttcagcatcatctggcgctagttctggtggcgccagtggcggctccggtggttctggcgaatctcaagttcggtcccagttcagcaaggatatcgaaaagctgctgaacgagcaagtgaacaaagagatgcagtccagcaacctgtatatgtccatgtccagctggtcctacacacactccctcgatggcgctggcctgttcctgtttgatcacgctgccgaggaatacgagcacgccaagaagctgatcatcttcctgaacgagaacaacgtgcccgtgcagctgacctccatctctgcccctgagcacaagttcgagggcctgacacagatctttcagaaggcctacgaacacgagcagcacatcagcgagtccatcaaccagatcgtggaccacgccatcaagtccaaggatcacgccaccttcaactttctgcagtggtacgtggccgagcagcatgaggaagaggtgctgttcaaggacatcctggacaagatcgagctgatcggccaagagaaccatggcctgtacctggccgaccagtacgtgaagggaatcgccaagtctcggaagtcctga

SEQ ID NO: 20 - DNA, 3972 bp, synthetic construct
  misc_feature 1..3972: note = Synthetic: Exemplary polynucleotide encoding a gL/gH/gp42/ferritin nanoparticle (e.g., C17 of SEQ ID NO: 10)
  source 1..3972: mol_type = other DNA
gccaccatgagagctgtgggagtgttcctggccatctgcctggtcaccatcttcgtgctgcctacctggggcaactgggcttacccttgctgtcatgtgacccagctgagagcccagcatctgctggccctggaaaacatctccgacatctacctggtgtccaaccagacctgcgacgccttctctctggcctctctgaactctcctaagcagggctccaaccagctcgtgatctccagatgtgccaacggcctgaacgtggtgtccttcttcatctctatcctgaagcggtccagcagcgccctgaccggacatctgagagagctgctgaccacactggaaaccctgtacggctccttcagcgtggaagatctgtttggcgcccagctgaacagatacgcctggcatagaggtggcggcggatctggctctgcttcttctggtgcttctgcctctggatcctctggcggctctggttccggttctggaagcggatcttctgccagctctggcgcatcttctggcggagctagcggaggctctggtggtagcggagctgcttctctgtccgaagtgaagctgcacctggacatcgagggccacgccagccactatacaatcccttggactgagctgctggccaaggtgccaggactgtctcctgaagctctttggcgcgaggccaacgtgaccgaggatctggcatctatgctgaaccggtacaagctgatctacaagaccagcggcaccctgggaatcgctctggctgagcctgtggatatccctgctgtgtccgagggctctatgcaggtcgacgcttctaaagtgcaccccggcgtgatcagcggcctgaattctcctgcctgtatgctgagcgcccctctggaaaagcagctgttctactacatcggcaccatgctgcccaacaccagacctcactcctacgtgttctaccagctgcggtgccacctgtcttacgtggccctgtctatcaacggcgacaagttccagtacaccggcgctatgacctccaagttcctgatgggcacctacaagagagtgaccgagaagggcgacgagcatgtgctgtctctggtgttcggaaagaccaaggacctgcctgacctgagaggccccttcagctatccctctctgacctccgctcagtccggcgagtattccctggtcatcgtgaccaccttcgtgcactacgccaacttccacaactacttcgtgcccaacctgaaggacatgttctctcgggccgtgacaatgaccgccgcctcttacgctagatacgtgctgcagaaactggtgctgctggaaatgaagggcggctgcagagagcccgagctggaaacagagacactgaccaccatgttcgaggtgtccgtggccttcttcaaagtgggccatgctgtgggcgagacaggcaacggatgtgtggatctgagatggctggctaagagcttcttcgagctgaccgtgctgaaggatatcatcggcatctgctacggcgccaccgtgaagggcatgcagtcttacggactggaaagactggccgccatcctgatggctaccgtgaagatggaagaactgggccacctgaccaccgagaagcaagagtacgctctgagactggccaccgtgggctatcctaaagccggcgtttactccggactgatcggcggagcaacatccgtgctgctgtccgcctacaacagacaccctctgttccagcctctgcacaccgtgatgcgcgagacactgttcatcggctctcacgtggtgctgcgcgagctgagactgaacgtgacaacccagggacctaatctggccctgtatcagctgctgagcaccgctctgtgtagcgctctggaaatcggcgaggtgctgagaggactggctctgggaacagagtccggcctgttctccccttgctacctgtctctgagattcgacctgaccagagataagctgctgtctatcgcccctcaagaggctaccctggatcaggctgcagtgtctcaggccgtggatggctttctgggcagactgtccctggaaagagaggacagggacgcttggcatctgcccgcctataagtgcgtggacagactggacaaggtgctgatgatcattcccctgatcaatgtgaccttcatcatctcctccgaccgggaagtgcgaggctctgctctgtatgaggcctctactacctacctgtcctccagcctgtttctgtcccctgtgatcatgaacaagtgctcccagggcgcagtggccggcgaacctagacagatccctaagatccagaacttcacccggactcagaagtcctgtatcttctgcggcttcgccctgctgtcctacgacgagaaagaaggcctggaaaccaccacctacatcaccagccaagaggtgcagaactccatcctgtccagcaattacttcgacttcgacaacctgcatgtgcactacctgctgctgactaccaacggcacagtgatggaaatcgccggcctgtacgaggaaagagcatccggcggaggttctggcagtgctagtagtggcgcttccgcttctggctcctccggctctggatctggaagtggctcctctagtgcttccagcggcgctatcacatgggtgcccaagcctaacgtggaagtgtggccagtggatcctcctccacctgtgaacttcaacaagaccgccgagcaagaatacggggacaaagaagtgaaactgccccactggacccctacactgcacacctttcaggtgccccagaactacaccaaggccaactgcacctactgcaatacccgcgagtacacctttagctacaagggctgctgcttctacttcaccaagaagaaacacacctggcagggctgctttcaggcctgtgctgagctgtacccctgtacctacttctacggccccacacctgacatcctgccagtggtcaccagaaacctgaacgccatcgagtccctgtgggtcggagtgtacagagtcggcgaaggcaactggacctctctggatggcggaaccttcaaggtgtaccagatcttcggctcccattgcacctacgtgtccaagttctccaccgtgcctgtgtctcaccacgagtgcagctttctgaagccctgcctgtgcgtgtcccagagatctaatagcggcggcagtggctctgcatctagtggcgcaagtgcttccggatcttccggctcaggctccggtagtggatcttctagtgcctcttccggcgcttctagcggtggtgcatctggtggatctggcggaagcggcggaggaagtggatcagcaagttcaggcgcttctgctagcggctctagtggcagtggcagcggttcaggatcttcctcagcaagcagcggagcatcaagtggtggtgccagcggaggatcaggcggttctggcgaatctcaagtgcggtcccagttcagcaaggatatcgaaaagctgctgaacgagcaagtgaacaaagagatgcagtccagcaacctgtatatgtccatgtccagctggtcctacacacactcccttgatggcgctggcctgttcctgtttgaccacgccgctgaggaatacgagcacgccaagaagctgatcatcttcctgaacgagaacaacgtgcccgtgcagctgacctccatctctgcccctgagcacaagttcgagggcctgacacagatctttcagaaggcctacgaacacgagcagcacatcagcgagtccatcaaccagatcgtggaccacgctatcaagtccaaggaccacgccaccttcaactttctgcagtggtacgtggccgagcagcatgaggaagaggtgctgttcaaggacatcctggacaagatcgagctgatcggccaagagaaccatggcctgtacctggccgaccagtacgtgaagggaatcgccaagtctcggaagtcctga

SEQ ID NO: 21 - DNA, 3972 bp, synthetic construct
  misc_feature 1..3972: note = Synthetic: Exemplary polynucleotide encoding a gL/gH/gp42/ferritin nanoparticle (e.g., C18 of SEQ ID NO: 11)
  source 1..3972: mol_type = other DNA
gccaccatgagagctgtgggagtgttcctggccatctgcctggtcaccatcttcgtgctgcctacctggggcaactgggcttacccttgctgtcatgtgacccagctgagagcccagcatctgctggccctggaaaacatctccgacatctacctggtgtccaaccagacctgcgacgccttctctctggcctctctgaactctcctaagcagggctccaaccagctcgtgatctccagatgtgccaacggcctgaacgtggtgtccttcttcatctctatcctgaagcggtccagcagcgccctgaccggacatctgagagagctgctgaccacactggaaaccctgtacggctccttcagcgtggaagatctgtttggcgcccagctgaacagatacgcctggcatagaggtggcggcggatctggctctgcttcttctggtgcttctgcctctggatcctctggcggctctggttccggttctggaagcggatcttctgccagctctggcgcatcttctggcggagctagcggaggctctggtggtagcggagctgcttctctgtccgaagtgaagctgcacctggacatcgagggccacgccagccactatacaatcccttggactgagctgctggccaaggtgccaggactgtctcctgaagctctttggcgcgaggccaacgtgaccgaggatctggcatctatgctgaaccggtacaagctgatctacaagaccagcggcaccctgggaatcgctctggctgagcctgtggatatccctgctgtgtccgagggctctatgcaggtcgacgcttctaaagtgcaccccggcgtgatcagcggcctgaattctcctgcctgtatgctgagcgcccctctggaaaagcagctgttctactacatcggcaccatgctgcccaacaccagacctcactcctacgtgttctaccagctgcggtgccacctgtcttacgtggccctgtctatcaacggcgacaagttccagtacaccggcgctatgacctccaagttcctgatgggcacctacaagagagtgaccgagaagggcgacgagcatgtgctgtctctggtgttcggaaagaccaaggacctgcctgacctgagaggccccttcagctatccctctctgacctccgctcagtccggcgagtattccctggtcatcgtgaccaccttcgtgcactacgccaacttccacaactacttcgtgcccaacctgaaggacatgttctctcgggccgtgacaatgaccgccgcctcttacgctagatacgtgctgcagaaactggtgctgctggaaatgaagggcggctgcagagagcccgagctggaaacagagacactgaccaccatgttcgaggtgtccgtggccttcttcaaagtgggccatgctgtgggcgagacaggcaacggatgtgtggatctgagatggctggctaagagcttcttcgagctgaccgtgctgaaggatatcatcggcatctgctacggcgccaccgtgaagggcatgcagtcttacggactggaaagactggccgccatgctgatggctaccgtgaagatggaagaactgggccacctgaccaccgagaagcaagagtacgctctgagactggccaccgtgggctatcctaaagccggcgtttactccggactgatcggcggagcaacatccgtgctgctgtccgcctacaacagacaccctctgttccagcctctgcacaccgtgatgcgcgagacactgttcatcggctctcacgtggtgctgcgcgagctgagactgaacgtgacaacccagggacctaatctggccctgtatcagctgctgagcaccgctctgtgtagcgctctggaaatcggcgaggtgctgagaggactggctctgggaacagagtccggcctgttctccccttgctacctgtctctgagattcgacctgaccagagataagctgctgtctatggcccctcaagaggctaccctggatcaggccgcagtgtctaatgccgtggatggctttctgggcagactgtccctggaaagagaggacagggacgcttggcatctgcccgcctataagtgcgtggacagactggacaaggtgctgatgatcattcccctgatcaatgtgaccttcatcatctcctccgaccgggaagtgcgaggctctgctctgtatgaggcctctactacctacctgtcctccagcctgtttctgtcccctgtgatcatgaacaagtgctcccagggcgcagtggccggcgaacctagacagatccctaagatccagaacttcacccggactcagaagtcctgtatcttctgcggcttcgccctgctgtcctacgacgagaaagaaggcctggaaaccaccacctacatcaccagccaagaggtgcagaactccatcctgtccagcaattacttcgacttcgacaacctgcatgtgcactacctgctgctgactaccaacggcacagtgatggaaatcgccggcctgtacgaggaaagagcatccggcggaggttctggcagtgctagtagtggcgcttccgcttctggctcctccggctctggatctggaagtggctcctctagtgcttccagcggcgctatcacatgggtgcccaagcctaacgtggaagtgtggccagtggatcctcctccacctgtgaacttcaacaagaccgccgagcaagaatacggggacaaagaagtgaaactgccccactggacccctacactgcacacctttcaggtgccccagaactacaccaaggccaactgcacctactgcaatacccgcgagtacacctttagctacaagggctgctgcttctacttcaccaagaagaaacacacctggaacggctgctttcaggcctgcgctgagctgtacccctgtacctacttctacggccccacacctgacatcctgccagtggtcaccagaaacctgaacgccatcgagtccctgtgggtcggagtgtacagagtcggcgaaggcaactggacctctctggatggcggcaccttcaaggtgtaccagatcttcggctcccattgcacctacgtgtccaagttctccaccgtgcctgtgtctcaccacgagtgcagctttctgaagccctgcctgtgcgtgtcccagagatctaatagcggcggcagtggctctgcatctagtggcgcaagtgcttccggatcttccggctcaggcagtggttctggctctagctcagcttcttccggcgcaagttcaggcggtgcatctggtggatctggcggaagcggcggaggaagtggatctgcttctagcggagcatccgcctccggatcatcaggatctggatccggtagcggctccagctctgcaagtagtggtgcctcaagcggaggtgccagcggtggcagcggaggatccggcgaatctcaagttcggtcccagttcagcaaggatatcgaaaagctgctgaacgagcaagtgaacaaagagatgcagtccagcaacctgtatatgtccatgtccagctggtcctacacacactcccttgatggcgctggcctgttcctgtttgaccacgccgctgaggaatacgagcacgccaagaaactgatcatcttcctgaacgagaacaacgtgcccgtgcagctgacctccatctctgcccctgagcacaagttcgagggcctgacacagatctttcagaaggcctacgaacacgagcagcacatcagcgagtccatcaaccagatcgtggaccacgctatcaagtccaaggaccacgccaccttcaactttctgcagtggtacgtggccgagcagcatgaggaagaggtgctgttcaaggacatcctggacaagatcgagctgatcggccaagagaaccatggcctgtacctggccgaccagtacgtgaagggaatcgccaagtctcggaagtcctga

SEQ ID NO: 22 - DNA, 1893 bp, synthetic construct
  misc_feature 1..1893: note = Synthetic: Exemplary polynucleotide encoding a gp220/ferritin nanoparticle (e.g., SEQ ID NO: 14)
  source 1..1893: mol_type = other DNA
atggattctaagggctccagccagaagggctctagactgctgctgctcctggtggtgtctaacctcctgttgcctcagggcgtgctggctgaagctgctctgctcgtgtgccagtacaccatccagagcctgatccatctgaccggcgaggaccctggcttcttcaacgtggaaatccctgagttccctttctaccctacctgcaacgtgtgcaccgccgacgtgaacgtgaccatcaactttgatgtcggcggcaagaagcaccagctggacctggattttggccagctgacccctcacaccaaggccgtgtatcaacctagaggcgcctttggcggctccgagaacgccaccaatctgtttctgctggaactgctcggagctggcgagctggctctgaccatgcggtctaagaaactgcccatcaatgtgaccacaggcgaggaacagcaggtttccctggaatccgtggacgtgtacttccaagacgtgttcggcaccatgtggtgccaccacgccgagatgcagaaccccgtgtatctgatccccgagacagtgccctacatcaagtgggacaactgcaactccaccaacatcaccgccgtcgtcagagcccaaggcctggatgttacactgcccctgagcctgcctacctccgctcaggactctaacttctccgtgaaaaccgagatgctgggcaacgagatcgacatcgagtgcatcatggaagatggcgagatctcccaggtgctgcccggcgacaacaagttcaatatcacctgttccggctacgagtctcacgtgccatctggcggcatcctgacctctacaagccctgtggctacccctattcctggcaccggctacgcttactccctgagactgacacctcggcctgtgtccagattcctgggcaacaactccatcctgtacgtgttctactccggcaacggccctaaggcttctggcggcgattactgcatccagtccaacatcgtgttctccgacgagatccccgccagccaggatatgcctaccaacaccaccgatatcacctacgtgggcgacaacgccacctactccgtgcctatggtcacctccgaggatgccaactctcccaacgtgaccgtgacagccttttgggcctggcctaacaacaccgagacagacttcaagtgcaagtggaccctgaccagcggcaccccatctggctgcgagaatatctctggcgccttcgcctctaaccggaccttcgacatcaccgtgtctggcctgggaaccgctcctaagacactgatcatcacccggaccgccacaaatgctaccaccaccactcacaaagtgatcttcagcaaggcccctgagggcagcgaatctcaagtgcggcagcagttctccaaggacatcgagaagctgctgaacgagcaagtgaacaaagagatgcagtcctccaacctgtatatgtccatgtcctcttggagctacacccacagcctggatggcgctggcctgttcctgtttgatcacgccgctgaggaatacgagcacgccaagaagctgatcatcttcctgaacgagaacaacgtgccagtgcagctgacctccatctctgccccagagcacaagttcgagggcctgacacagatcttccagaaggcctacgaacacgaacagcacatctccgagtccatcaacaatatcgtggaccacgccattaagtgcaaggatcacgccaccttcaactttctgcagtggtacgtggccgagcagcacgaggaagaggtgctgtttaaggacatcctggacaagatcgagctgatcggcaatgagaaccacggcctgtacctggccgaccagtacgtgaagggaatcgccaagagccggaagtcctga

SEQ ID NO: 23 - protein, 166 aa, synthetic construct
  REGION 1..166: note = Synthetic: Exemplary H. pylori ferritin
FSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 24 - protein, 173 aa, synthetic construct
  REGION 1..173: note = Synthetic: Exemplary Bullfrog H. pylori ferritin
ESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 25 - protein, 600 aa, synthetic construct
  REGION 1..600: note = Synthetic: Gp220 with ferritin of SEQ ID NO: 24
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPEGSESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 26 - protein, 4 aa, synthetic construct
  REGION 1..4: note = Synthetic: 4 amino acid linker
GGGS

SEQ ID NO: 27 - protein, 8 aa, synthetic construct
  REGION 1..8: note = Synthetic: 8 amino acid linker
GGGSGGGS

SEQ ID NO: 28 - protein, 20 aa, synthetic construct
  REGION 1..20: note = Synthetic: 20 amino acid linker
GGGSGGGSGGGSGGGSGGGS

SEQ ID NO: 29 - protein, 47 aa, synthetic construct
  REGION 1..47: note = Synthetic: 47 amino acid linker
SGGGSGSASSGASASGSSCSGSGSGSSSASSGASSGGASGGGSGGSG

SEQ ID NO: 30 - protein, 46 aa, synthetic construct
  REGION 1..46: note = Synthetic: 46 amino acid linker
GGSGSASSGASASGSSNGSGSGSGSNSSASSGASSGGASGGSGGSG

SEQ ID NO: 31 - protein, 16 aa, synthetic construct
  REGION 1..16: note = Synthetic: 16 amino acid linker
GGGGSGGGGSGGGGSG

SEQ ID NO: 32 - protein, 28 aa, synthetic construct
  REGION 1..28: note = Synthetic: 28 amino acid linker
GGSGSGSNSSASSGASSGGASGGSGGSG

SEQ ID NO: 33 - protein, 26 aa, synthetic construct
  REGION 1..26: note = Synthetic: 26 amino acid linker (FR1 linker)
GGSGSASAEAAAKEAAAKAGGSGGSG

SEQ ID NO: 34 - protein, 32 aa, synthetic construct
  REGION 1..32: note = Synthetic: 32 amino acid linker (FR2 linker)
GGSGSASAEAAAKEAAAKEAAAKASGGSGGSG

SEQ ID NO: 35 - protein, 630 aa, synthetic construct
  REGION 1..630: note = Synthetic: gp350D123(gp220 bfpFerr Nanoparticle N19Q/C31S/S111C)
MDSKGSSQKGSRLLLLLVVSNLLLPQGVLAEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPEGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 36 - protein, 1321 aa, synthetic construct
  REGION 1..1321: note = Synthetic: C16 gL/gH/gp42/ferritin including the leader of SEQ ID NO: 12
MRAVGVFLAICLVTIFVLPTWGNWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAILMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSIAPQEATLDQAAVSQAVDGFLGRLSLEREDRDAWHLPAYKCVDRLDKVLMIIPLINVTFIISSDREVRGSALYEASTTYLSSSLFLSPVILNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWQGCFQACAELYPCTYFYGPTPDILPVVTRSLQAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 37 - protein, 1329 aa, synthetic construct
  REGION 1..1329: note = Synthetic: C16 gL/gH/gp42/ferritin including the leader of SEQ ID NO: 13
MDSKGSSQKGSRLLLLLVVSNLLLPQGVLANWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAILMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSIAPQEATLDQAAVSQAVDGFLGRLSLEREDRDAWHLPAYKCVDRLDKVLMIIPLINVTFIISSDREVRGSALYEASTTYLSSSLFLSPVILNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWQGCFQACAELYPCTYFYGPTPDILPVVTRSLQAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 38 - protein, 1321 aa, synthetic construct
  REGION 1..1321: note = Synthetic: C17 gL/gH/gp42/ferritin including the leader of SEQ ID NO: 12
MRAVGVFLAICLVTIFVLPTWGNWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAILMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSIAPQEATLDQAAVSQAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWQGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 39 - protein, 1329 aa, synthetic construct
  REGION 1..1329: note = Synthetic: C17 gL/gH/gp42/ferritin including the leader of SEQ ID NO: 13
MDSKGSSQKGSRLLLLLVVSNLLLPQGVLANWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAILMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSIAPQEATLDQAAVSQAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWQGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 40 - protein, 1321 aa, synthetic construct
  REGION 1..1321: note = Synthetic: C18 gL/gH/gp42/ferritin including the leader of SEQ ID NO: 12
MRAVGVFLAICLVTIFVLPTWGNWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 41 - protein, 1329 aa, synthetic construct
  REGION 1..1329: note = Synthetic: C18 gL/gH/gp42/ferritin including the leader of SEQ ID NO: 13
MDSKGSSQKGSRLLLLLVVSNLLLPQGVLANWAYPCCHVTQLRAQHLLALENISDIYLVSNQTCDAFSLASLNSPKQGSNQLVISRCANGLNVVSFFISILKRSSSALTGHLRELLTTLETLYGSFSVEDLFGAQLNRYAWHRGGGGSGSASSGASASGSSGGSGSGSGSGSSASSGASSGGASGGSGGSGAASLSEVKLHLDIEGHASHYTIPWTELLAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGEYSLVIVTTFVHYANFHNYFVPNLKDMFSRAVTMTAASYARYVLQKLVLLEMKGGCREPELETETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERASGGGSGSASSGASASGSSGSGSGSGSSSASSGAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGGSGSASSGASASGSSGSGSGSGSSSASSGASSGGASGGSGGSGGGSGSASSGASASGSSGSGSGSGSSSASSGASSGGASGGSGGSGESQVRSQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 42 - protein, 166 aa, Helicobacter pylori
  SITE 1..166: note = Exemplary H. pylori ferritin
FSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

SEQ ID NO: 43 - protein, 166 aa, Helicobacter pylori
  SITE 1..166: note = Exemplary H. pylori ferritin
FSKDIEKLLNEQVNKEMNSSNLYMSMSSWSYTHSLDGAGLFLFDHAAEEYEHAKKLIIFLNENNVPVQLTSISAPEHKFEGLTQIFQKAYEHEQHISESINQIVDHAIKSKDHATFNFLQWYVAEQHEEEVLFKDILDKIELIGQENHGLYLADQYVKGIAKSRKS

(19) United States
(12) Patent Application Publication
(10) Pub. No.: US 2025/0281605 A1
(43) Pub. Date: Sep. 11, 2025

(54) Antigenic Epstein Barr Virus Polypeptides

(71) Applicant: Sanofi, Paris (FR)

(72) Inventors: Te-Hui Chou, Wayland, MA (US); Gary J. Nabel, Delray Beach, FL (US); Laura Nguyen, Medford, MA (US); Chih-Jen Wei, Weston, MA (US)

(73) Assignee: Sanofi, Paris (FR)

(21) Appl. No.: 19/073,654

(22) Filed: Mar. 7, 2025

(30) Foreign Application Priority Data
Mar. 8, 2024 (EP) EP24305361.8

(51) Int. Cl. A61K 39/245; A61K 38/00; A61K 39/00; A61K 47/64; A61P 31/22; C07K 14/005; C07K 14/205; C07K 14/46; C12N 5/071; C12N 7/00

(52) U.S. Cl. CPC A61K 39/245; A61K 47/64; A61P 31/22; C07K 14/005; C07K 14/205; C07K 14/463; C12N 5/0682; C12N 7/00; A61K 38/00; A61K 2039/55555; A61K 2039/70; C07K 2319/00; C12N 2501/06; C12N 2710/16222; C12N 2710/16234; C12N 2710/16251

(57) ABSTRACT

This disclosure relates to fusion proteins comprising one or more EBV polypeptides and a ferritin protein, and their use in eliciting antibodies against EBV.

### CRADA STATEMENT

This invention was created in the performance of a Cooperative Research and Development Agreement with the National Institutes of Health, an Agency of the Department of Health and Human Services. The Government of the United States has certain rights in this invention.

### CROSS-REFERENCE TO RELATED APPLICATIONS

This application claims the benefit of European Patent Application No. 24305361.8, filed Mar. 8, 2024, the entire contents of which are incorporated herein by reference.

### SEQUENCE LISTING

The instant application contains a Sequence Listing which has been submitted electronically in XML format and is hereby incorporated by reference in its entirety. Said XML copy, created on Feb. 26, 2025, is named “01121-0054-00PCT.xml” and is 85,801 bytes in size.

### INTRODUCTION

Even with many successes in the field of vaccinology, new breakthroughs are needed to protect humans against many life-threatening infectious diseases. Many currently licensed vaccines rely on decade-old technologies to produce live-attenuated or inactivated killed pathogens, which carry inherent safety concerns, and in many cases stimulate only short-lived, weak immune responses that require the administration of multiple doses. While advances in genetic and biochemical engineering have made it possible to develop therapeutic agents to challenging disease targets, these applications to the field of vaccinology have not been fully realized. Recombinant protein technologies now allow the design of optimal antigens. Additionally, nanoparticles have increasingly demonstrated the potential for optimal antigen presentation and targeted drug delivery. Nanoparticles with multiple attached antigens have been shown to have increased binding avidity afforded by the multivalent display of their molecular cargos, and an ability to cross biological barriers more efficiently due to their nanoscopic size. Helicobacter pylori (H. pylori) ferritin nanoparticles fused to influenza virus haemagglutinin (HA) protein has allowed improved antigen stability and increased immunogenicity in mouse influenza models (see Kanekiyo et al., Nature 499:102-106 (2013)). This fusion protein self-assembled into an octahedrally-symmetric nanoparticle and presented 8 trimeric HA spikes to give a robust immune response in various pre-clinical models when used with an adjuvant.

Epstein Barr virus (EBV) infects about 95% of the adult population worldwide and has been known to be associated with two B-cell lymphomas, Burkitt's and Hodgkin's lymphomas. The virus can also infect epithelial cells and is associated with nasopharyngeal cancer. Furthermore, EBV causes most cases of infectious mononucleosis in developed countries, affecting mainly children and young adults. Infectious mononucleosis can result in a long recovery period of up to one month. There are currently no approved vaccines on the market, so there is a strong need for a preventive vaccine.

### SUMMARY

It is an object of this disclosure to provide compositions, kits, methods, and uses that can provide one or more of the advantages discussed above, or at least provide the public with a useful choice. Accordingly, the following embodiments are disclosed herein.

Embodiment 1 is a fusion protein comprising:
(a) an Epstein Barr virus (EBV) polypeptide comprising: (i) an EBV gH polypeptide and/or (ii) an EBV gL polypeptide; and
(b) a ferritin of SEQ ID NO: 1 comprising at least one of the following substitutions: (i) a glutamine at a residue corresponding to position N102 of SEQ ID NO: 1; (ii) a glutamine at a residue corresponding to position N145 of SEQ ID NO: 1; or (iii) a combination thereof.

Embodiment 2 is the fusion protein of embodiment 1, wherein the EBV polypeptide further comprises an EBV gp42 polypeptide.

Embodiment 3 is a fusion protein comprising:
(a) an Epstein Barr virus (EBV) polypeptide comprising: (i) an EBV gp42 polypeptide and (ii) an EBV gH polypeptide and/or an EBV gL polypeptide; and
(b) a ferritin of SEQ ID NO: 1 comprising at least one of the following substitutions: (i) a glutamine at a residue corresponding to position N102 of SEQ ID NO: 1; (ii) a glutamine at a residue corresponding to position N145 of SEQ ID NO: 1; or (iii) a combination thereof.

Embodiment 4 is the fusion protein of any one of embodiments 1-3, wherein the ferritin comprises a glutamine at a residue corresponding to position N102 of SEQ ID NO: 1 and a glutamine at a residue corresponding to position N145 of SEQ ID NO: 1, optionally wherein the ferritin further comprises a glutamine at a residue corresponding to position N18 of SEQ ID NO: 1 and a serine at a residue corresponding to position C30 of SEQ ID NO: 1.

Embodiment 5 is the fusion protein of any one of embodiments 1-4, wherein a bullfrog ferritin extension sequence is linked at the N-terminus of the ferritin, wherein the bullfrog ferritin extension sequence comprises the amino acid sequence of SEQ ID NO: 2.

Embodiment 6 is the fusion protein of any one of embodiments 1-5, wherein the EBV polypeptide comprises: (i) an EBV gp42 polypeptide, (ii) an EBV gH polypeptide, and (iii) an EBV gL polypeptide.

Embodiment 7 is the fusion protein of embodiment 6, wherein the EBV gL polypeptide, the EBV gH polypeptide, the EBV gp42 polypeptide, and the ferritin are arranged in N-terminal to C-terminal order.

Embodiment 8 is the fusion protein of any one of embodiments 1-7, further comprising a linker between each EBV polypeptide.

Embodiment 9 is the fusion protein of embodiment 8, wherein the linker between the EBV gL polypeptide and the EBV gH polypeptide is 44 to 48 amino acids, and the linker between the EBV gH polypeptide and the EBV gp42 polypeptide is 32 amino acids.

Embodiment 10 is the fusion protein of embodiment 9, wherein the linker between the EBV gL polypeptide and the EBV gH polypeptide is 46 amino acids, and the linker between the EBV gH polypeptide and the EBV gp42 polypeptide is 32 amino acids.

Embodiment 11 is the fusion protein of embodiment 8, wherein a linker separates the EBV gL polypeptide and the EBV gH polypeptide and consists of an amino acid sequence that has at least 80% identity to SEQ ID NO: 3; and/or wherein a linker separates the EBV gH polypeptide and the EBV gp42 polypeptide and consists of an amino acid sequence that has at least 80% identity to SEQ ID NO: 4.

Embodiment 12 is the fusion protein of any one of embodiments 1-11, further comprising a further linker that separates the ferritin and the EBV gp42 polypeptide.

Embodiment 13 is the fusion protein of embodiment 12, wherein the further linker has a length of 88 amino acids.

Embodiment 14 is the fusion protein of embodiment 13, wherein the further linker consists of an amino acid sequence having at least 80% identity to SEQ ID NO: 5.

Embodiment 15 is the fusion protein of any one of embodiments 1-14, wherein the ferritin comprises the amino acid sequence of SEQ ID NO: 23.

Embodiment 16 is the fusion protein of any one of embodiments 1-15, wherein the EBV gp42 polypeptide comprises an amino acid sequence having at least 80%, 90%, 95%, 96%, 97%, 98%, or 99% identity to residues 856-1038 of any one of SEQ ID NOs: 9-11.

Embodiment 17 is the fusion protein of embodiment 16, wherein the EBV gH polypeptide comprises an amino acid sequence having at least 80%, 90%, 95%, 96%, 97%, 98%, or 99% identity to residues 162-823 of any one of SEQ ID NOs: 9-11; optionally wherein the EBV gH polypeptide comprises a leucine at a residue corresponding to position 189 of any one of SEQ ID NOs: 9-11; a glutamic acid at a residue corresponding to position 368 of any one of SEQ ID NOs:9-11; and a glutamic acid at a residue corresponding to position 429 of any one of SEQ ID NOs: 9-11.

Embodiment 18 is the fusion protein of embodiment 16 or 17, wherein the EBV gL polypeptide comprises an amino acid sequence having at least 80%, 90%, 95%, 96%, 97%, 98%, or 99% identity to residues 1-115 of any one of SEQ ID NOs: 9-11; optionally wherein the EBV gL polypeptide comprises an alanine at a residue corresponding to position 36 of any one of SEQ ID NOs: 9-11; a glutamine at a residue corresponding to position 47 of any one of SEQ ID NOs: 9-11; and a glutamine at a residue corresponding to position 105 of any one of SEQ ID NOs: 9-11.

Embodiment 19 is the fusion protein of any one of embodiments 1-18, comprising a sequence having at least 90%, 95%, 96%, 97%, 98%, or 99% identity to (a) amino acid residues 1-1133 of any one of SEQ ID NOs: 9-11, (b) amino acid residues 1-1148 of any one of SEQ ID NOs: 36, 38, or 40, or (c) amino acid residues 1-1156 of any one of SEQ ID NOs: 37, 39, or 41.

Embodiment 20 is the fusion protein of any one of embodiments 1-19, optionally comprising a mammalian leader sequence positioned at the N-terminus of the fusion protein, optionally wherein the fusion protein comprises an EBV gL polypeptide, an EBV gH polypeptide, an EBV gp42 polypeptide, and a ferritin, wherein the EBV gL polypeptide, the EBV gH polypeptide, the EBV gp42 polypeptide, and the ferritin are arranged in N-terminal to C-terminal order within the EBV polypeptide, and the mammalian leader sequence is positioned N-terminal to the EBV gL polypeptide.

Embodiment 21 is the fusion protein of embodiment 20, having the mammalian leader sequence, wherein the mammalian leader sequence is selected from SEQ ID NO: 12 or SEQ ID NO: 13.

Embodiment 22 is a fusion protein comprising:
(a) an Epstein Barr virus (EBV) polypeptide comprising an EBV gL polypeptide, and an EBV gH polypeptide, wherein the EBV gL polypeptide comprises the sequence of residues 1-115 of SEQ ID NO: 11 and the EBV gH polypeptide comprises the sequence of residues 162-823 of SEQ ID NO: 11;
(b) a ferritin polypeptide of SEQ ID NO: 1, or a ferritin polypeptide having SEQ ID NO: 1 and at least one mutation selected from one or more of a glutamine at a residue corresponding to position N18 of SEQ ID NO: 1; a serine at a residue corresponding to position C30 of SEQ ID NO: 1; glutamine at a residue corresponding to position N102 of SEQ ID NO: 1; and a glutamine at a residue corresponding to position N145 of SEQ ID NO: 1; and
(c) a bullfrog ferritin extension sequence that is linked at the N-terminus of the ferritin, wherein the bullfrog ferritin extension sequence comprises the amino acid sequence of SEQ ID NO: 2, and further optionally a mammalian leader sequence positioned at the N-terminus of the fusion protein, wherein the mammalian leader sequence comprises the amino acid sequence of SEQ ID NO: 12 or SEQ ID NO: 13;
wherein the EBV gL polypeptide, the EBV gH polypeptide, and the ferritin are arranged in N-terminal to C-terminal order within the EBV polypeptide, and the optional mammalian leader sequence is positioned N-terminal to the EBV gL polypeptide.

Embodiment 23 is a fusion protein comprising:
(a) an Epstein Barr virus (EBV) polypeptide comprising an EBV gL polypeptide, an EBV gH polypeptide, and an EBV gp42 polypeptide, wherein the EBV gL polypeptide comprises the amino acid sequence of residues 1-115 of SEQ ID NO: 11, the EBV gH polypeptide comprises the amino acid sequence of residues 162-823 of SEQ ID NO: 11, and the EBV gp42 polypeptide comprises the amino acid sequence of residues 856-1038 of SEQ ID NO: 11;
(b) a ferritin comprising SEQ ID NO: 24; and
(c) optionally a mammalian leader sequence positioned at the N-terminus of the fusion protein, wherein the mammalian leader sequence comprises the amino acid sequence of SEQ ID NO: 12 or SEQ ID NO: 13;
wherein the EBV gL polypeptide, the EBV gH polypeptide, the EBV gp42 polypeptide and the ferritin are arranged in N-terminal to C-terminal order within the EBV polypeptide, and the optional mammalian leader sequence is positioned N-terminal to the EBV gL polypeptide.

Embodiment 24 is the fusion protein of embodiment 22 or 23, wherein a linker separates the EBV gL polypeptide and the EBV gH polypeptide and comprises the amino acid sequence of SEQ ID NO: 3.

Embodiment 25 is the fusion protein of any one of embodiments 23-24, wherein a linker separates the EBV gH polypeptide and the EBV gp42 polypeptide and comprises the amino acid sequence of SEQ ID NO: 4.

Embodiment 26 is the fusion protein of any one of embodiments 23-25, wherein a linker separates the EBV gp42 polypeptide and the ferritin and comprises the amino acid sequence of SEQ ID NO: 5.

Embodiment 27 is the fusion protein of any one of embodiments 22-26, wherein the ferritin consists of the amino acid sequence of SEQ ID NO: 24.

Embodiment 28 is a fusion protein comprising the amino acid sequence of any one of SEQ ID NOs: 9-11 or 36-41.

Embodiment 29 is a fusion protein consisting of the amino acid sequence of any one of SEQ ID NOs: 9-11 or 36-41.

Embodiment 30 is a fusion protein comprising (i) an EBV gp220 polypeptide that comprises an amino acid sequence having at least 80%, at least 85%, at least 90%, at least 95%, at least 96%, at least 97%, at least 98%, at least 99%, or 100% sequence identity to residues 1-425 of SEQ ID NO: 14 and (ii) a ferritin comprising the amino acid sequence of any one of SEQ ID NOs: 23, 24, 42, and 43.

Embodiment 31 is the fusion protein of embodiment 30, wherein the amino acid sequence of the fusion protein consists of SEQ ID NO: 25 or 35.

Embodiment 32 is a ferritin nanoparticle comprising the fusion protein of any one of embodiments 1-29.

Embodiment 33 is a composition or kit comprising a first ferritin nanoparticle of embodiment 32 and optionally a second ferritin nanoparticle comprising a second fusion protein comprising an EBV gp220 polypeptide and a ferritin.

Embodiment 34 is the composition or kit of embodiment 33, wherein the EBV gp220 polypeptide comprises an amino acid sequence having at least 80%, at least 85%, at least 90%, at least 95%, at least 96%, at least 97%, at least 98%, at least 99%, or 100% sequence identity to residues 1-425 of SEQ ID NO: 14.

Embodiment 35 is the composition or kit of embodiment 33 or 34, wherein the ferritin in the second fusion protein comprises a serine or cysteine at a residue corresponding to position 110 of SEQ ID NO: 1; optionally wherein the ferritin comprises an amino acid sequence having at least 80% identity to residues 435-600 of SEQ ID NO:14.

Embodiment 36 is the composition or kit of embodiment 33, wherein the second fusion protein comprises an amino acid sequence having at least 80%, at least 85%, at least 90%, at least 95%, at least 96%, at least 97%, at least 98%, at least 99%, or 100% sequence identity to SEQ ID NO: 14, optionally wherein position C544 of SEQ ID NO: 14 is substituted to serine.

Embodiment 37 is a composition or kit comprising a first ferritin nanoparticle comprising a fusion protein consisting of the amino acid sequence of any one of SEQ ID NOs: 9-11 and (a) a second ferritin nanoparticle comprising a second fusion protein consisting of the amino acid sequence of SEQ ID NO: 14, or (b) a second ferritin nanoparticle comprising a second fusion protein consisting of the amino acid sequence of SEQ ID NO: 14 with a substitution to a serine at position C544 of SEQ ID NO: 14.

Embodiment 38 is a composition or kit comprising a first ferritin nanoparticle comprising a fusion protein consisting of the amino acid sequence of SEQ ID NO: 11 and a second ferritin nanoparticle comprising a second fusion protein consisting of the amino acid sequence of SEQ ID NO: 14.

Embodiment 39 is a pharmaceutical composition comprising the fusion protein of any one of embodiments 1-31, or ferritin nanoparticle of embodiment 32, or the composition of any one of embodiments 33-38; and a pharmaceutically acceptable carrier.

Embodiment 40 is the pharmaceutical composition of embodiment 39, wherein the pharmaceutically acceptable carrier comprises an adjuvant.

Embodiment 41 is a bivalent vaccine comprising the composition of any one of embodiments 33-38, wherein the fusion protein and second fusion protein are present in a 1:1 ratio by weight or wherein the fusion protein and second fusion protein are present in a 1:1 molar ratio.

Embodiment 42 is the fusion protein of any one of embodiments 1-31, the composition of any one of embodiments 33-38, the ferritin particle of embodiment 32, the pharmaceutical composition of embodiment 39 or embodiment 40, or the bivalent vaccine of embodiment 41 for use in a method of eliciting an immune response to EBV in a subject, in protecting a subject against infection with EBV, or in preventing an EBV-associated disease in a subject.

Embodiment 43 is a method of eliciting an immune response to EBV in a subject, protecting a subject against infection with EBV or preventing an EBV-associated disease in a subject, comprising administering to the subject the fusion protein of any one of embodiments 1-31, the composition of any one of embodiments 33-38, the ferritin particle of embodiment 32, the pharmaceutical composition of embodiment 39 or embodiment 40, or the bivalent vaccine of embodiment 41.

Embodiment 44 is a nucleic acid encoding the fusion protein of any one of embodiments 1-31.

Embodiment 45 is an expression vector comprising the nucleic acid of embodiment 44; and optionally further comprising a nucleic acid encoding the second fusion protein of the composition of any one of embodiments 32-37.

Embodiment 46 is a host cell comprising the nucleic acid of embodiment 44 or the expression vector of embodiment 45.

Embodiment 47 is a method of preparing the fusion protein according to any one of embodiments 1 to 31, comprising: culturing the host cell according to embodiment 46 under conditions whereby the fusion protein is expressed in the host cell, and recovering the fusion protein from the cultured host cell culture.

Embodiment 48 is the method of embodiment 47, wherein the host cells are Chinese hamster ovary cells.

Embodiment 49 is the method of embodiment 47 or embodiment 48, wherein the host cells are dihydrofolate reductase (DHFR) deficient; and the expression vector further comprises a polynucleotide sequence that encodes DHFR.

Embodiment 50 is the method of embodiment 49, comprising culturing the host cells in the presence of a DHFR-selection agent; optionally wherein the DHFR-selection agent comprises methotrexate.

Embodiment 51 is the method of any one of embodiments 47-50, wherein the expression vector comprises a polynucleotide sequence that encodes the amino acid sequence of any one of SEQ ID NOs: 9-11 or 36-41.

Embodiment 52 is the method of embodiment 51, wherein the expression vector comprises the polynucleotide sequence of any one of SEQ ID NOs: 19-22.

Embodiment 53 is a fusion protein obtainable by or produced by the method of any one of embodiments 47-52.

Embodiment 54 is a ferritin protein consisting of the amino acid sequence of SEQ ID NO: 24.

Additional objects and advantages will be set forth in the description which follows, and/or will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate certain embodiments and together with the description, serve to explain the principles described herein.

### BRIEF DESCRIPTION OF THE DRAWINGS

FIGS. 1A-B show clone titers and viable packaging cell densities (VCDs) for EBV Ferritin gp220 on day 5 of batch culture. Clones were seeded in 10 mL batch cultures in spin tubes at a seeding density of 1×10⁶ vc/mL. The batch culture medium was chemically-defined (CD) CHO/30% (v/v) Efficient Feed B, supplemented with 4 mM L-glutamine. Media harvests were collected at day 5 (or day 6 for the six clones shown as white bars in FIG. 1A) and analyzed using the Vi-Cell XR for viable cell density determination. A separate harvest aliquot was clarified by centrifugation and the clarified harvests were assayed for titer using the Octet method (Octet Red 96). The black bar shows the titer of the respective presort pool at day 5 of batch culture. FIG. 1A shows the A and H clone sets; FIG. 1B shows the B and I clone sets.

FIG. 2 shows fed batch clone screen productivity results. The fed batch clone screen was run using the Ambr® 15-mL system (Sartorius Stedim) using in-house basal and feed media. For each clone, cells were inoculated into an Ambr reactor to achieve a target seed density of 1×10⁶ viable cells/mL in a total working volume of 14.8 mL. Daily media feeds were initiated on day 2. From day 2 onward, the cultures were sampled for daily viable cell density and percent viability measurements (Vi-Cell XR). The cultures were sampled on days 5, 7, and 12 for titer analysis; these samples were clarified by centrifugation and frozen at −80° C. for storage. After the run was completed (Day 12), the clarified harvests were thawed and assayed for titer using the Octet method (Octet Red 96). For each clone, the average specific productivity (through day 7) was determined using the change in titer divided by the change in integrated viable cell concentration (IVCC) between days 0 and 7. Culture titers are shown as bars (Black—Day 5; White—Day 7; Striated—Day 12), and average specific productivity is shown as a single point for each clone.

FIGS. 3A-C show octet analysis of a C18 gL/gH/gp42 nanoparticle (SEQ ID NO: 11) binding to antibodies confirming antigen epitopes. FIGS. 3A-C show binding curves of the C18 gL/gH/gp42 nanoparticle using Ammol antibody (FIG. 3A), E1D1 antibody (FIG. 3B), and 1D8 antibody (FIG. 3C), respectively. Each antibody binds a different epitope on the C18 antigen.

FIGS. 4A-4B show ELISAs of serum from mice immunized at day 0 and day 21 with 1 μg of either C18 gL/gH/gp42 ferritin nanoparticle (FIG. 4A, SEQ ID NO: 11) or a gp350D123 (FIG. 4B, SEQ ID NO: 14) ferritin nanoparticle. The ELISA plate was coated with His-tagged antigen monomer. The control was the pre-bleed of the mice before immunization. There were 5 mice in each group.

FIGS. 5A-B show electron microscopy negative stain analysis of nanoparticles as assessed in Example 5. FIG. 5A shows the gL/gH/gp42 C18 ferritin nanoparticle (SEQ ID NO: 11). FIG. 5B shows the gp350D123 ferritin nanoparticle (SEQ ID NO: 14). Uniform particle formation can be seen, with few aggregates. Magnification: 73,000×. Scale bar: 200 nm.

### DETAILED DESCRIPTION

Fusion proteins comprising one or more EBV polypeptides and a ferritin protein of the invention, are provided, which can be antigenic when administered alone, with adjuvant as a separate molecule, and/or as part of a nanoparticle (e.g., ferritin particle). Such fusion proteins and compositions comprising such fusion proteins can be used to elicit antibody responses against Epstein Barr virus (EBV). The fusion proteins can comprise an EBV polypeptide (such as a gL, gH, gL/gH, gp220, or gp42 polypeptide, or combinations thereof) and a ferritin multimerization domain. In some embodiments, the ferritin comprises or consists of the amino acids of any one of SEQ ID NOs: 23, 24, 42, and 43. In some embodiments, the ferritin comprises or consists of the amino acids of SEQ ID NO: 23. In some embodiments, the ferritin comprises or consists of the amino acids of SEQ ID NO: 24. In some embodiments, the ferritin comprises or consists of the amino acids of SEQ ID NO: 42. In some embodiments, the ferritin comprises or consists of the amino acids of SEQ ID NO: 43. Nucleic acids that encode the fusion proteins described herein are also provided.

### A. Definitions

As used herein, an “EBV polypeptide” refers to a polypeptide comprising all or part of an amino acid sequence encoded by EBV. Similarly, gL, gH, gp42, and gp220 polypeptides refer to polypeptides comprising all or part of a gL, gH, gp42, or gp220 amino acid sequence, respectively, encoded by EBV. Polypeptides with, e.g., at least 80% identity to an EBV-encoded polypeptide will necessarily comprise part of the EBV-encoded polypeptide. The terms “gL polypeptide,” “gH polypeptide,” “gp42 polypeptide,” and “gp220 polypeptide” are used interchangeably with “EBV gL polypeptide,” “EBV gH polypeptide,” “EBV gp42 polypeptide,” and “EBV gp220 polypeptide,” respectively. Immunization with an EBV polypeptide as part or all of an antigenic polypeptide may confer protection from infection with EBV. Unless the context dictates otherwise, any polypeptide disclosed herein comprising an EBV polypeptide can comprise all or part of multiple sequences encoded by EBV (for example, all or part of gL and gH of EBV, or all or part of gL, gH, and gp42 of EBV).

As used herein, a “monomer,” or “monomer construct” refers to a construct expressed as a single-chain protein. A monomer may comprise gL and gH of EBV expressed in a single chain, or gL, gH, and gp42 of EBV expressed in a single chain.

“Ferritin” or “ferritin protein,” as used herein, refers to a protein with detectable sequence identity to H. pylori ferritin (e.g., SEQ ID NO: 1, 23, 24, 42, or 43) In particular embodiments, the ferritin comprises or consists of the amino acid sequence of SEQ ID NO: 23 or 24. In other particular embodiments, the ferritin comprises or consists of the amino acid sequence of SEQ ID NO: 42 or 43.

“Wild-type ferritin,” as used herein, refers to a ferritin whose sequence comprises a naturally occurring sequence.

As used herein, a “ferritin monomer” refers to a single ferritin molecule (or, where applicable, a single ferritin heavy or light chain) that has not assembled with other ferritin molecules. A “ferritin multimer” comprises multiple associated ferritin monomers. A “ferritin protein” includes monomeric ferritin and multimeric ferritin.

As used herein, “ferritin particle,” refers to ferritin that has self-assembled into a globular form. Ferritin particles are sometimes referred to as “ferritin nanoparticles” or simply “nanoparticles”. In some embodiments, a ferritin particle comprises 24 ferritin monomers (or, where applicable, 24 total heavy and light chains).

“Hybrid ferritin,” as used herein, refers to ferritin comprising H. pylori ferritin with an amino terminal extension of bullfrog ferritin. An exemplary sequence used as an amino terminal extension of bullfrog ferritin appears as SEQ ID NO: 2. In hybrid ferritin, the amino terminal extension of bullfrog ferritin can be fused to H. pylori ferritin (See SEQ ID NO: 24). “Bullfrog ferritin extension sequence,” as used herein, is a polypeptide comprising the sequence of SEQ ID NO: 2. Hybrid ferritin is also sometimes referred to as “bfpFerr” or “bfp ferritin.” Any of the constructs comprising a bullfrog ferritin extension sequence can be provided without the sequence. Exemplary bullfrog ferritin extension sequences are provided in Table 2. Where Table 2 shows a bullfrog ferritin extension sequence, the same construct may be made without the sequence.

“N-glycan,” as used herein, refers to a saccharide chain attached to a protein at the amide nitrogen of an N (asparagine) residue of the protein. As such, an N-glycan is formed by the process of N-glycosylation. This glycan may be a polysaccharide.

“Glycosylation,” as used herein, refers to the addition of a saccharide unit to a protein.

“Immune response,” as used herein, refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a “protective immune response” refers to an immune response that protects a subject from infection (e.g., prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production, and the like. An “antibody response” is an immune response in which antibodies are produced.

As used herein, an “antigen” refers to an agent that elicits an immune response, and/or an agent that is bound by a T cell receptor (e.g., when presented by an MIC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens can include antigenic ferritin proteins comprising ferritin (e.g., comprising one or more mutations) and a non-ferritin polypeptide as described herein.

“Adjuvant,” as used herein, refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules. An adjuvant may be administered as a separate molecule in a composition or covalently bound (conjugated) to ferritin or a fusion protein (such as an antigenic ferritin polypeptide) disclosed herein.

A “fusion protein,” as used herein, refers to a protein comprising two or more of the polypeptides disclosed herein (such as two or more of a gL, gH, gp220, gp42 polypeptide, ferritin, or combinations thereof). A fusion protein may be a monomer construct (i.e., a construct expressed as a single-chain protein) but may also be in multimer form. As disclosed herein, a fusion protein may comprise additional components, such as, but not limited to, one or more linkers separating two or more of the polypeptides. A fusion protein may comprise or consist of an “antigenic polypeptide,” an “antigenic EBV polypeptide,” or an “antigenic ferritin polypeptide” as disclosed herein.

An “antigenic EBV polypeptide” is used herein to refer to a polypeptide comprising all or part of an EBV amino acid sequence of sufficient length that the molecule is antigenic with respect to EBV. Antigenicity may be a feature of the EBV sequence as part of a construct further comprising a heterologous sequence, such as a ferritin. That is, if an EBV sequence is part of a construct further comprising a heterologous sequence, then it is sufficient that the construct can serve as an antigen that generates anti-EBV antibodies, regardless of whether the EBV sequence without the heterologous sequence could do so.

“Antigenic ferritin polypeptide” and “antigenic ferritin protein” are used interchangeably herein to refer to a polypeptide comprising a ferritin and a non-ferritin polypeptide (e.g., an EBV polypeptide) of sufficient length that the molecule is antigenic with respect to the non-ferritin polypeptide. Antigenicity may be a feature of the non-ferritin sequence as part of the larger construct. That is, it is sufficient that the construct can serve as an antigen against the non-ferritin polypeptide, regardless of whether the non-ferritin polypeptide without the ferritin could do so. In some embodiments, the non-ferritin polypeptide is an EBV polypeptide, in which case the antigenic ferritin polypeptide is also an “antigenic EBV polypeptide.”

“Antigenic polypeptide” is used herein to refer to a polypeptide which is either or both of an antigenic ferritin polypeptide and an antigenic EBV polypeptide. In some embodiments, the ferritin of an antigenic ferritin polypeptide comprises or consists of the amino acids of any one of SEQ ID NOs: 23, 24, 42, and 43.

A “surface-exposed” amino acid, as used herein, refers to an amino acid residue in a protein (e.g., a ferritin) with a side chain that can be contacted by solvent molecules when the protein is in its native three-dimensional conformation after multimerization, if applicable. Thus, for example, in the case of ferritin that forms a 24-mer, a surface-exposed amino acid residue is one whose side chain can be contacted by solvent when the ferritin is assembled as a 24-mer, e.g., as a ferritin multimer or ferritin particle.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. The terms “individual” or “patient” are used and are intended to be interchangeable with a human subject.

As used herein, the term “vaccination” or “vaccinate” refers to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

The disclosure describes nucleic acid sequences and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

As used herein, a “host cell” (or a “population of host cells”) refers to any cell or population of cells of any organism that is used for the purpose of producing a recombinant protein encoded by an expression vector or propagating the expression vector introduced into the host cell. A “recombinant cell” refers to a host cell that comprises a heterologous expression vector, which may or may not be integrated into the host cell chromosome. In some embodiments, a host cell has been transfected with an expression vector comprising a polynucleotide sequence that encodes a fusion protein disclosed herein, and is capable of producing a fusion protein as disclosed herein. Accordingly, a host cell useful in the present disclosure can include the progeny of the original cell if such cell has been transformed by the nucleic acid. A variety of suitable host cells (such as host cells disclosed herein) are useful herein.

The term “vector” or “expression vector” refers to a recipient nucleic acid molecule modified to comprise or incorporate a provided nucleic acid sequence, such as a nucleic acid sequence encoding a fusion protein disclosed herein. One exemplary type of vector of use herein is a “plasmid,” which refers to a circular double stranded DNA molecule into which additional DNA may be ligated. Another exemplary type of vector of use herein is a viral vector (such as an adeno associated viral vector), wherein additional DNA segments may be ligated into the viral genome.

As used herein, a cell (such as a population of host cells) has been “transfected” with a nucleic acid, e.g., with an expression vector comprising a polynucleotide sequence that encodes a fusion protein disclosed herein, when such nucleic acids have been introduced inside the cell. A cell may be transfected, e.g., with one or more (such as one, two, three, or four) expression vectors or other nucleic acids through any process known in the art, including but not limited to electroporation, calcium phosphate precipitation, or contacting with a polynucleotide-liposome complex. The term “transfection,” as used herein, encompasses any means of introducing one or more (such as one, two, three, or four) nucleic acids inside a cell.

### B. Fusion Proteins Comprising an EBV Polypeptide and Ferritin

In some embodiments, a fusion protein is provided, comprising an EBV polypeptide and ferritin, optionally wherein the ferritin comprises or consists of the sequence of any one of SEQ ID NOs: 23, 24, 42, and 43, optionally comprising a bullfrog ferritin extension sequence at the N-terminus of the ferritin (such as the bullfrog ferritin extension sequence of SEQ ID NO: 2). The EBV polypeptide can be any of the EBV polypeptides described herein, such as a gL, gH, gL/gH, gL/gH/gp42, gp220, gp42 polypeptide, or combinations thereof. In such embodiments, a fusion protein may be a monomer construct (i.e., a construct expressed as a single-chain protein). A monomer construct may comprise any one or more of the polypeptides disclosed herein (such as gL, gH, gL/gH, gL/gH/gp42, gp220, gp42 polypeptide) and a ferritin of the invention expressed in a single chain. As disclosed elsewhere herein, a fusion protein may comprise additional components, such as, but not limited to, one or more linkers disclosed herein, separating two or more of the polypeptides. In some embodiments, a fusion protein of the present disclosure comprises or consists of an antigenic polypeptide, an antigenic EBV polypeptide, and/or an antigenic ferritin polypeptide.

In some embodiments, the fusion protein comprises an amino acid sequence having at least 80%, at least 85%, at least 90%, at least 95%, at least 96%, at least 97%, at least 98%, at least 99%, or 100% identity to residues 1-1133 of any one of SEQ ID NOs: 9-11, and a ferritin of the present invention. In some embodiments, the fusion protein comprises an amino acid sequence having at least 80%, at least 85%, at least 90%, at least 95%, at least 96%, at least 97%, at least 98%, at least 99%, or 100% sequence identity to of any one of SEQ ID NOS: 9-11, 14, 25, or 35-41, wherein the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 9, wherein the ferritin comprises or consists of the amino acids of SEQ ID NO: 23 or 24. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 10, wherein the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 11, wherein the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 25, wherein the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 36, and the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 37, and the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 38, and the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 39, and the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 40, and the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 41, and the ferritin comprises or consists of the amino acids of any one of SEQ ID NOs: 23, 24, 42, and 43. In some embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 14 or 35, wherein the ferritin comprises or consists of the amino acids of any one of SEQ ID NOs: 23, 24, 42, and 43. In some embodiments, the fusion protein comprises the amino acid sequence of any one of SEQ ID NOs: 9-11, 25, or 35. In particular embodiments, the fusion protein comprises an amino acid sequence having at least 80%, at least 85%, at least 90%, at least 95%, at least 96%, at least 97%, at least 98%, at least 99%, or 100% sequence identity to SEQ ID NO: 14 or 35, optionally wherein the ferritin is the ferritin of SEQ ID NO: 14 with a substitution to serine at residue C544 or is the ferritin of SEQ ID NO: 35 with a substitution to serine at residue C574. Additional fusion proteins and components thereof are disclosed in International Application No. PCT/US2019/025419, which is incorporated by reference herein in its entirety.

In some embodiments, the ferritin is H. pylori ferritin (see SEQ ID NOs: 1, 23, 24, 42, or 43 for an exemplary H. pylori ferritin sequence).

In some embodiments, a ferritin nanoparticle is provided comprising a fusion protein as disclosed herein comprising an EBV polypeptide and a ferritin, wherein the ferritin comprises or consists of the amino acids of SEQ ID NO: 23 or 24. In some embodiments, a ferritin nanoparticle is provided comprising a fusion protein as disclosed herein comprising an EBV polypeptide and a ferritin, wherein the ferritin comprises or consists of the amino acids of SEQ ID NO: 42 or 43.

In some embodiments, a bullfrog ferritin extension sequence is linked at the N-terminus of the ferritin. In some embodiments, the bullfrog ferritin extension sequence that is linked at the N-terminus of the ferritin comprises an amino acid sequence having at least 80%, at least 85%, at least 90%, at least 95%, at least 96%, at least 97%, at least 98%, at least 99%, or 100% to SEQ ID NO: 2. In some embodiments, a bullfrog ferritin extension sequence that is linked at the N-terminus of the ferritin comprises the amino acid sequence of SEQ ID NO: 2.

In particular embodiments, the fusion protein comprises an EBV polypeptide comprising: (i) an EBV gH polypeptide and/or (ii) an EBV gL polypeptide; and a ferritin comprising the amino acid sequence of SEQ ID NO: 1; and further comprising at least one of: (i) a glutamine at a residue corresponding to position N102 of SEQ ID NO: 1; (ii) a glutamine at a residue corresponding to position N145 of SEQ ID NO: 1; and/or (iii) a bullfrog ferritin extension sequence that is linked at the N-terminus of the ferritin, wherein the bullfrog ferritin extension comprises the amino acid sequence of SEQ ID NO: 2. In particular embodiments, the fusion protein comprises (a) an Epstein Barr virus (EBV) polypeptide comprising: (i) an EBV gH polypeptide and/or (ii) an EBV gL polypeptide; and (b) a ferritin of SEQ ID NO: 1 comprising at least one of the following substitutions: (i) a glutamine at a residue corresponding to position N102 of SEQ ID NO: 1; (ii) a glutamine at a residue corresponding to position N145 of SEQ ID NO: 1; or (iii) a combination thereof. In some such embodiments, the fusion protein further comprises an EBV gp42 polypeptide. In particular embodiments, a fusion protein comprises (a) an Epstein Barr virus (EBV) polypeptide comprising: (i) an EBV gp42 polypeptide and (ii) an EBV gH polypeptide and/or an EBV gL polypeptide; and (b) a ferritin of SEQ ID NO: 1 comprising at least one of the following substitutions: (i) a glutamine at a residue corresponding to position N102 of SEQ ID NO: 1; (ii) a glutamine at a residue corresponding to position N145 of SEQ ID NO: 1; and (iii) a bullfrog ferritin extension sequence that is linked at the N-terminus of the ferritin, wherein the bullfrog ferritin extension sequence comprises the amino acid sequence of SEQ ID NO: 2.

In some embodiments, the ferritin comprises a glutamine at a residue corresponding to position N102 of SEQ ID NO: 1 and a glutamine at a residue corresponding to position N145 of SEQ ID NO: 1, optionally wherein the ferritin further comprises a glutamine at a residue corresponding to position N18 of SEQ ID NO: 1 and a serine at a residue corresponding to position C30 of SEQ ID NO: 1.

In other particular embodiments, the ferritin comprises a glutamine at a residue corresponding to position N102 of SEQ ID NO: 1, a glutamine at a residue corresponding to position N145 of SEQ ID NO: 1, and a glutamine at a residue corresponding to position N18 of SEQ ID NO: 1.

In some embodiments, a fusion protein, comprises (i) an EBV gp42 polypeptide, (ii) an EBV gH polypeptide, and (iii) an EBV gL polypeptide, optionally wherein the EBV gL polypeptide, the EBV gH polypeptide, the EBV gp42 polypeptide, and the ferritin are arranged in N-terminal to C-terminal order within the fusion protein. In some such embodiments, the fusion protein comprises one or more, or each, of a linker between the EBV gL polypeptide and the EBV gH polypeptide, between the EBV gH polypeptide and the EBV gp42 polypeptide, and between the EBV gp42 polypeptide and the ferritin. In some embodiments, the linker has a length of at least 15 amino acids, as disclosed elsewhere herein. In such embodiments, each linker may be independently selected from any linker disclosed herein, such as a linker having at least 80%, at least 85%, at least 90%, at least 95%, at least 96%, at least 97%, at least 98%, at least 99%, or 100% sequence identity to any one of SEQ ID Nos: 3-8 or 26-34. In a particular embodiment, the linker between the EBV gL polypeptide and the EBV gH polypeptide is 44 to 48 amino acids in length, and the linker between the EBV gH polypeptide and the EBV gp42 polypeptide is 32 amino acids in length. In another particular embodiment, the linker between the EBV gL polypeptide and the EBV gH polypeptide is 46 amino acids in length, and the linker between the EBV gH polypeptide and the EBV gp42 polypeptide is 32 amino acids in length. In some embodiments, the fusion protein lacks one or more of the linkers (such as one or more of a linker between the EBV gL polypeptide and the EBV gH polypeptide, between the EBV gH polypeptide and the EBV gp42 polypeptide, and/or between the EBV gp42 polypeptide and the ferritin). In particular embodiments, a linker separates the EBV gL polypeptide and the EBV gH polypeptide and comprises an amino acid sequence having at least 80% identity to SEQ ID NO: 3; and/or a linker separates the EBV gH polypeptide and the EBV gp42 polypeptide and comprises an amino acid sequence having at least 80% identity to SEQ ID NO: 4. In particular embodiments, a linker separates the EBV gL polypeptide and the EBV gH polypeptide and consists of an amino acid sequence that has at least 80% identity to SEQ ID NO: 3; and/or a linker separates the EBV gH polypeptide and the EBV gp42 polypeptide and consists of an amino acid sequence that has at least 80% identity to SEQ ID NO: 4. In some embodiments, a linker separates the EBV gp42 polypeptide and the ferritin and comprises an amino acid sequence having at least 80% identity to SEQ ID NO: 5. In some embodiments, the linker separating the EBV gp42 polypeptide and the ferritin comprises the amino acid sequence of SEQ ID NO: 5. In some embodiments, the linker separating the EBV gp42 polypeptide and the ferritin consists of the amino acid sequence of SEQ ID NO: 5.

In particular embodiments, the fusion protein comprises the amino acid sequence of any one of SEQ ID Nos: 9-11, 14, 25, or 35-41. In other particular embodiments, the fusion protein consists of the amino acid sequence of any one of SEQ ID Nos: 9-11, 14, 25, or 35-41.

In some embodiments, the fusion protein comprises a mammalian leader sequence positioned at the N-terminus of the fusion protein, optionally wherein the fusion protein comprises an EBV gL polypeptide, an EBV gH polypeptide, an EBV gp42 polypeptide, and a ferritin of the present invention, wherein the EBV gL polypeptide, the EBV gH polypeptide, the EBV gp42 polypeptide, and the ferritin are arranged in N-terminal to C-terminal order within the EBV polypeptide, and the mammalian leader sequence is positioned N-terminal to the EBV gL polypeptide. Any of the disclosed constructs comprising a mammalian leader sequence can be provided without the sequence. Exemplary mammalian leader sequences are provided in Table 2. Where Table 2 shows a mammalian leader sequence, the same construct may be made without the sequence.

In some embodiments, a composition comprises (a) a first fusion protein comprising an EBV gL polypeptide, an EBV gH polypeptide, and/or an EBV gp42 polypeptide, and a ferritin of the present invention, and (b) a second fusion protein comprising an EBV gp220 polypeptide and optionally a ferritin. In some embodiments, the first fusion protein comprises an amino acid sequence having at least 80%, at least 85%, at least 90%, at least 95%, at least 96%, at least 97%, at least 98%, at least 99%, or 100% identity to residues 1-1133 of any one of SEQ ID NOs: 9-11, and a ferritin of the present invention. In some embodiments, the fusion protein comprises an amino acid sequence having at least 80%, at least 85%, at least 90%, at least 95%, at least 96%, at least 97%, at least 98%, at least 99%, or 100% sequence identity to of any one of SEQ ID NOS: 9-11 or 36-41, wherein the ferritin comprises or consists of the amino acids of any one of SEQ ID NOs: 23, 24, 42, and 43. In some embodiments, the EBV gp220 polypeptide of the second fusion protein comprises an amino acid sequence having at least 80%, at least 85%, at least 90%, at least 95%, at least 96%, at least 97%, at least 98%, at least 99%, or 100% sequence identity to residues 1-425 of SEQ ID NO: 14, 25, or 35, and optionally a ferritin. In some embodiments, the gp220 polypeptide further comprises a mammalian leader sequence linked at the N-terminus of the gp220 polypeptide, optionally wherein the leader sequence comprises the amino acid sequence of SEQ ID NO: 12 or SEQ ID NO: 13. In particular embodiments, the second fusion protein further comprises a ferritin. In particular embodiments, the ferritin comprises a serine or cysteine at a residue corresponding to position 110 of SEQ ID NO: 1; optionally wherein the ferritin comprises an amino acid sequence having at least 80%, at least 85%, at least 90%, at least 95%, at least 96%, at least 97%, at least 98%, at least 99%, or 100% sequence identity to residues 435-600 of SEQ ID NO: 14. In particular embodiments, the second fusion protein comprises an amino acid sequence having at least 80%, at least 85%, at least 90%, at least 95%, at least 96%, at least 97%, at least 98%, at least 99%, or 100% sequence identity to SEQ ID NO: 14, 25, or 35. In some embodiments, the ferritin in the second fusion protein comprises a serine or cysteine at a residue corresponding to position 110 of SEQ ID NO: 1. In some such embodiments, the ferritin comprises an amino acid sequence having at least 80% identity to residues 435-600 of SEQ ID NO: 14. In a particular embodiment, the composition comprises a first fusion protein consisting of the amino acid sequence of SEQ ID NO: 11 and a second fusion protein consisting of the amino acid sequence of SEQ ID NO: 14.

In some embodiments comprising a second fusion protein, the first fusion protein and second fusion protein are present in a 1:1, 1:2, 1:3, 1:4, 1:5, 1:6, 1:7, 1:8, 1:9, 1:10, 2:1, 3:1, 4:1, 5:1, 6:1, 7:1, 8:1, 9:1, or 10:1 ratio by weight or wherein the first fusion protein and second fusion protein are present in a 1:1, 1:2, 1:3, 1:4, 1:5, 1:6, 1:7, 1:8, 1:9, 1:10, 2:1, 3:1, 4:1, 5:1, 6:1, 7:1, 8:1, 9:1, or 10:1 molar ratio.

In particular embodiments, the fusion protein comprises an EBV polypeptide comprising an EBV gL polypeptide, and an EBV gH polypeptide, wherein the EBV gL polypeptide comprises the sequence of residues 1-115 of SEQ ID NO: 11 and the EBV gH polypeptide comprises the sequence of residues 162-823 of SEQ ID NO: 11; a ferritin polypeptide of SEQ ID NO: 1, or a ferritin polypeptide of SEQ ID NO: 1 and at least one mutation selected from any one or more of a glutamine at a residue corresponding to position N18 of SEQ ID NO: 1; a serine at a residue corresponding to position C30 of SEQ ID NO: 1; glutamine at a residue corresponding to position N102 of SEQ ID NO: 1; a glutamine at a residue corresponding to position N145 of SEQ ID NO: 1; and optionally a bullfrog ferritin extension sequence that is linked at the N-terminus of the ferritin, wherein the bullfrog ferritin extension sequence comprises the amino acid sequence of SEQ ID NO: 2, and further optionally a mammalian leader sequence positioned at the N-terminus of the fusion protein, wherein the mammalian leader sequence comprises the amino acid sequence of SEQ ID NO: 12 or 13. In such embodiments, the EBV gL polypeptide, the EBV gH polypeptide, and the ferritin are arranged in N-terminal to C-terminal order within the fusion protein, and the optional mammalian leader sequence is positioned N-terminal to the EBV gL polypeptide. In some such embodiments, a linker separates the EBV gL polypeptide and the EBV gH polypeptide and comprises the amino acid sequence of SEQ ID NO: 3. Some such embodiments further comprise a second fusion protein comprising an EBV gp220 polypeptide and ferritin, and the second fusion protein comprises the amino acid sequence of SEQ ID NO: 14 or 35, optionally wherein the ferritin comprises or consists of the amino acid sequence of any one of SEQ ID NOs: 23, 24, 42, and 43. In a particular embodiment, the second fusion protein comprises an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 35. In some embodiments wherein the second fusion protein comprises an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 14 or 35, the second fusion protein further comprises a serine at residue C544 or SEQ ID NO: 14 or at residue C574 of SEQ ID NO: 35.

In other particular embodiments, the fusion protein comprises an EBV polypeptide comprising an EBV gL polypeptide, an EBV gH polypeptide, and an EBV gp42 polypeptide, wherein the EBV gL polypeptide comprises the amino acid sequence of residues 1-115 of SEQ ID NO: 11, the EBV gH polypeptide comprises the amino acid sequence of residues 162-823 of SEQ ID NO: 11, and the EBV gp42 polypeptide comprises the amino acid sequence of residues 856-1038 of SEQ ID NO: 11; a ferritin comprising SEQ ID NO: 24; and optionally a mammalian leader sequence positioned at the N-terminus of the fusion protein, wherein the mammalian leader sequence comprises the amino acid sequence of SEQ ID NO: 12 or 13. In such embodiments, the EBV gL polypeptide, the EBV gH polypeptide, the EBV gp42 polypeptide, and the ferritin are arranged in N-terminal to C-terminal order within the fusion protein, and the optional mammalian leader sequence is positioned N-terminal to the EBV gL polypeptide. In some such embodiments, a linker separates the EBV gL polypeptide and the EBV gH polypeptide and comprises the amino acid sequence of SEQ ID NO: 3. In some such embodiments, a linker separates the EBV gH polypeptide and the EBV gp42 polypeptide and comprises the amino acid sequence of SEQ ID NO: 4. Some such embodiments further comprise a second fusion protein comprising an EBV gp220 polypeptide and ferritin, and the second fusion protein comprises the amino acid sequence of SEQ ID NO: 14, 25 or 35, optionally wherein the ferritin comprises or consists of the amino acid sequence of any one of SEQ ID NOs: 23, 24, 42, and 43.

### 1. Ferritin Mutations

The present disclosure provides a H. Pylori ferritin sequence of SEQ ID NO: 1 comprising at least one of the following substitutions: (i) a glutamine at a residue corresponding to position N102 of SEQ ID NO: 1; (ii) a glutamine at a residue corresponding to position N145 of SEQ ID NO: 1; or (iii) a combination thereof. In some embodiments, a ferritin of the present disclosure comprises a mammalian leader sequence that is linked at the N-terminus of the ferritin. In some such embodiments, a ferritin of the present disclosure comprises a bullfrog ferritin extension sequence that is linked at the N-terminus of the ferritin, the bullfrog ferritin extension sequence optionally comprising the amino acid sequence of SEQ ID NO: 2. In some embodiments, a ferritin comprises a glutamine at a residue corresponding to position N102 of SEQ ID NO: 1 and a glutamine at a residue corresponding to position N145 of SEQ ID NO: 1. In some such embodiments, the ferritin further comprises a glutamine at a residue corresponding to position N18 of SEQ ID NO: 1 and a serine at a residue corresponding to position C30 of SEQ ID NO: 1 In some embodiments, the ferritin comprises or consists of the amino acids of any one of SEQ ID NOs: 23, 23, 42, or 43.

### 2. Removal of Internal Cysteine

In some embodiments, the ferritin comprises a mutation replacing an internal cysteine with a non-cysteine amino acid. Removal of a native internal cysteine residue can ensure that there is only one unpaired cysteine per ferritin monomer and avoid undesired reactions such as disulfide formation. In some embodiments, C30 of the H. pylori ferritin of SEQ ID NO: 1 is replaced with a non-cysteine amino acid. In some embodiments, C30 of the H. pylori ferritin of SEQ ID NO: 1 is replaced with a serine (C30S), although any non-cysteine residue may be used, e.g., alanine, glycine, threonine, or asparagine. Analogous amino acids can be found in non-H. pylori ferritin by pair-wise or structural alignment. Thus, in some embodiments, the internal cysteine that is replaced in favor of non-cysteine is an amino acid residue that aligns with C30 of the H. pylori ferritin of SEQ ID NO: 1. An exemplary ferritin sequence showing a C30S mutation are shown in SEQ ID NO: 23. In some embodiments, when more than one internal cysteine is present in ferritin, two or more (e.g., each) internal cysteine is replaced with a non-cysteine amino acid, such as serine or an amino acid selected from serine, alanine, glycine, threonine, or asparagine.

### 3. Glycosylation

Human-compatible glycosylation can contribute to safety and efficacy in recombinant drug products. Regulatory approval may be contingent on demonstrating appropriate glycosylation as a critical quality attribute (see Zhang et al., Drug Discovery Today 21(5):740-765 (2016)). N-glycans can result from glycosylation of asparagine side chains and can differ in structure between humans and other organisms such as bacteria and yeast. Thus, it may be desirable to reduce or eliminate non-human glycosylation and/or N-glycan formation in ferritin according to the disclosure. In some embodiments, controlling glycosylation of ferritin improves the efficacy and/or safety of the composition, especially when used for human vaccination.

In some embodiments, ferritin is mutated to inhibit formation of an N-glycan. In some embodiments, a mutated ferritin has reduced glycosylation as compared to its corresponding wild type ferritin.

In some embodiments, the ferritin comprises a mutation replacing a surface-exposed asparagine with a non-asparagine amino acid. In some embodiments, the surface-exposed asparagine is N18 of the H. pylori ferritin of SEQ ID NO: 1 or a position that corresponds to position 18 of the H. pylori ferritin of SEQ ID NO: 1 as determined by pair-wise or structural alignment. In some embodiments, mutating such an asparagine, e.g., N18 of H. pylori ferritin, decreases glycosylation of ferritin. In some embodiments, the mutation replaces the asparagine with a glutamine. In some embodiments, the ferritin is an H. pylori ferritin comprising an N18Q mutation. Thus, in some embodiments, the ferritin comprises a glutamine at a residue corresponding to position 18 of SEQ ID NO: 1.

A mammal exposed to a glycosylated protein produced in bacteria or yeast may generate an immune response to the glycosylated protein, because the pattern of glycosylation of a given protein in bacteria or yeast could be different from the pattern of glycosylation of the same protein in a mammal. Thus, some glycosylated therapeutic proteins may not be appropriate for production in bacteria or yeast.

In some embodiments, decreased glycosylation of ferritin by amino acid mutation facilitates protein production in bacteria or yeast. In some embodiments, decreased glycosylation of ferritin reduces the potential for adverse effects in mammals upon administration of mutated ferritin that is expressed in bacteria or yeast. In some embodiments, the reactogenicity in a human subject of a mutated ferritin produced in bacteria or yeast is lower because glycosylation is decreased. In some embodiments, the incidence of hypersensitivity responses in human subjects is lower following treatment with a mutated ferritin with reduced glycosylation compared to wildtype ferritin.

In some embodiments, degradation in a subject of a composition comprising a mutated ferritin with reduced glycosylation is slower compared with a composition comprising a wild-type ferritin, or a composition comprising a corresponding ferritin with wild-type glycosylation. In some embodiments, a composition comprising a mutated ferritin with reduced glycosylation has reduced clearance in a subject compared with a composition comprising a wild-type ferritin, or a composition comprising a corresponding ferritin with wild-type glycosylation. In some embodiments, a composition comprising a mutated ferritin with reduced glycosylation has a longer-serum half-life compared to wild-type ferritin, or a composition comprising a corresponding ferritin with wild-type glycosylation.

### 4. Combinations of Mutations

In some embodiments, a ferritin comprises more than one type of mutation described herein. In some embodiments, the ferritin comprises one or more mutations independently selected from: a mutation to decrease glycosylation, a mutation to remove an internal cysteine, and a mutation to generate a surface-exposed cysteine. In some embodiments, the ferritin comprises a mutation to decrease glycosylation, a mutation to remove an internal cysteine, and a mutation to generate a surface-exposed cysteine. In particular embodiments, the ferritin comprises a serine at position 110 corresponding to the amino acid of SEQ ID NO: 1. In some embodiments, the ferritin comprises or consists of the amino acids of SEQ ID NO: 23. In some embodiments, the ferritin comprises or consists of the amino acids of SEQ ID NO: 24. In some embodiments, the ferritin comprises or consists of the amino acids of SEQ ID NO: 42. In some embodiments, the ferritin comprises or consists of the amino acids of SEQ ID NO: 43.

Positions of ferritin mutations in this paragraph refer to positions corresponding to a position of SEQ ID NO: 1 unless otherwise stated. A position in an amino acid sequence “corresponds” to a given position in SEQ ID NO: 1 if it aligns to that position according to a standard sequence alignment algorithm such as the Smith-Waterman algorithm using default parameters. In some embodiments, the ferritin comprises an N18Q mutation and a C30S mutation. In some embodiments, the ferritin comprises an N18Q mutation, a C30S mutation, and an E11C mutation. In some embodiments, the ferritin comprises an N18Q mutation, a C30S mutation, and an S71C mutation. In some embodiments, the ferritin comprises an N18Q mutation, a C30S mutation, and an A74C mutation. In some embodiments, the ferritin comprises an N18Q mutation, a C30S mutation, and a K79C mutation. In some embodiments, the ferritin comprises an N18Q mutation, a C30S mutation, and an S99C mutation. In some embodiments, the ferritin comprises an N18Q mutation, a C30S mutation, and an S110C mutation. In some embodiments, the ferritin does not comprise an S110C mutation. In some embodiments, the ferritin comprises an N18Q mutation, a C30S mutation, an N102Q mutation, and an N145Q mutation. In some embodiments, the ferritin comprises mutations corresponding to any of the foregoing sets of mutations, wherein the corresponding mutations change an N to a Q or a C to an S at positions determined by pair-wise alignment of the ferritin amino acid sequence to an H. pylori ferritin amino acid sequence (SEQ ID NOs: 1, 23, 24, 42, or 43). An exemplary ferritin comprising more than one type of mutation (e.g., an N18Q mutation, a C30S mutation, an N102Q mutation, and an N145Q mutation) is provided in SEQ ID NOs: 23, 24, 42, and 43.

In some particular embodiments, the ferritin comprises or consists of the amino acid sequence of SEQ ID NO: 23. In some particular embodiments, the ferritin comprises or consists of the amino acid sequence of residues 1134-1299 of any one of SEQ ID Nos: 9-11. In some embodiments, the ferritin comprises a glutamine at a residue corresponding to position 1151 of SEQ ID Nos: 9-11; a serine at a residue corresponding to position 1163 of any one of SEQ ID Nos: 9-11; a glutamine at a residue corresponding to position 1235 of any one of SEQ ID Nos: 9-11; and a glutamine at a residue corresponding to position 1278 of any one of SEQ ID Nos: 9-11.

### C. Fusion Proteins Comprising gL and gH Polypeptides and a Ferritin

EBV has three glycoproteins, glycoprotein B (gB), gH, and gL, that form the core membrane fusion machinery to allow viral penetration into a cell. gL and gH have been previously described, for example, in Matsuura et al., Proc Natl Acad Sci USA. 2010 Dec. 28; 107(52):22641-6. Monomers and trimers of gL and gH for use as vaccines have been described, for example, in Cui et al., Vaccine. 2016 Jul. 25; 34(34):4050-5. The gH and gL proteins associate to form a heterodimeric complex considered necessary for efficient membrane fusion and binding to epithelial cell receptors required for viral entry.

The fusion proteins disclosed herein can comprise EBV gL and EBV gH, and a ferritin disclosed herein (such as a ferritin of any one of SEQ ID NOs: 23, 24, 42, and 43). In some embodiments, the fusion protein forms a nanoparticle (e.g., ferritin particle), e.g., through multimerization of a ferritin. In some embodiments, a fusion protein according to this disclosure comprises an EBV gL polypeptide and an EBV gH polypeptide, and a linker having a length of at least 15 amino acids separating the EBV gL polypeptide and the EBV gH polypeptide. It has been found that a relatively long linker can provide benefits such as improved expression and/or immunogenicity. In some embodiments, a linker separates the EBV gL polypeptide and the EBV gH polypeptide and has a length of at least 15 amino acids, such as 15-100 amino acids, such as 15-90, 15-80, 15-70, 15-60, 15-50, 15-40, 15-30, or 15-20 amino acids. Such a linker may have a length of 15 to 60 amino acids, 20 to 60 amino acids, 30 to 60 amino acids, 40 to 60 amino acids, 30 to 50 amino acids, 30 to 40 amino acids, 44 to 48 amino acids, 44 amino acids, 46 amino acids, 48 amino acids, or 40 to 50 amino acids. In some embodiments, a linker separating the EBV gL polypeptide and the EBV gH polypeptide has at least 80%, at least 85%, at least 90%, at least 95%, at least 96%, at least 97%, at least 98%, at least 99%, or 100% identity to any one of SEQ ID Nos: 3-8 and 29-30. In a particular embodiment, a linker separating the EBV gL polypeptide and the EBV gH polypeptide has at least 80%, at least 85%, at least 90%, at least 95%, at least 96%, at least 97%, at least 98%, at least 99%, or 100% identity to SEQ ID NO: 3.

In some embodiments, the EBV gH and/or gL polypeptides are full-length gH and/or gL (for exemplary full-length sequences, see GenBank Accession Nos. CEQ35765.1 and YP_001129472.1, respectively). In some embodiments, the EBV gH and/or gL polypeptides are fragments of gH and/or gL. In some embodiments, the gL polypeptide is a gL(D7) construct with a 7-amino acid deletion at the end of the gL C-terminus. In some embodiments, the gH polypeptide comprises a mutation at C137, such as a C137A mutation. In some embodiments, the C137 mutation removes a native, unpaired cysteine to avoid non-specific conjugation. In some embodiments, the gH polypeptide comprises a mutation to remove a cysteine corresponding to cysteine 137 of SEQ ID NO: 17, such as a C137A mutation. In some embodiments, the C137 mutation removes a native, unpaired cysteine to avoid non-specific conjugation.

In some embodiments, the EBV gL polypeptide comprises an amino acid sequence with at least 80%, at least 85%, at least 90%, at least 95%, at least 96%, at least 97%, at least 98%, at least 99%, or 100% identity to SEQ ID NO: 16. In some embodiments, the EBV gH polypeptide comprises an amino acid sequence with at least 80%, at least 85%, at least 90%, at least 95%, at least 96%, at least 97%, at least 98%, at least 99%, or 100% identity to SEQ ID NO: 17. In some embodiments, the EBV gL polypeptide comprises an amino acid sequence having at least 80%, at least 85%, at least 90%, at least 95%, at least 96%, at least 97%, at least 98%, at least 99%, or 100% identity to residues 1-115 of any one of SEQ ID NOs: 9-11. In some embodiments, the EBV gH polypeptide comprises an amino acid sequence having at least 80%, at least 85%, at least 90%, at least 95%, at least 96%, at least 97%, at least 98%, at least 99%, or 100% identity to residues 162-823 of any one of SEQ ID NOs: 9-11.

In some embodiments, a fusion protein may comprise, in N- to C-terminal order, a gL polypeptide, a gH polypeptide, and a ferritin of the present invention. Stated differently, in some embodiments, an EBV gL polypeptide, an EBV gH polypeptide, and a ferritin of the present invention are arranged in N-terminal to C-terminal order within the fusion protein as a single chain. In some embodiments, the ferritin comprises or consists of the amino acids of SEQ ID NO: 23 or 24. In some embodiments, the ferritin comprises or consists of the amino acids of SEQ ID NO: 42 or 43.

In some embodiments, a mammalian leader sequence (also known as a signal sequence) is appended N-terminally to an EBV polypeptide, such as a gH or gL polypeptide, e.g., at the N-terminus of the EBV polypeptide (e.g., at the N-terminus of a fusion protein comprising the EBV polypeptide). In some embodiments, a mammalian leader sequence results in secretion of a disclosed fusion protein when expressed in mammalian cells. In some embodiments, the mammalian leader sequence comprises an amino acid sequence with at least 80%, at least 85%, at least 90%, at least 95%, at least 96%, at least 97%, at least 98%, at least 99%, or 100% identity to SEQ ID NOs: 12 or 13.

Native EBV gH and/or gL sequences are shown in GenBank Accession No. NC_009334.1 (Human herpesvirus 4, complete genome, dated 26 Mar. 2010). For some of the constructs disclosed herein, amino acids 23-137 of the gL amino acid sequence in NC_009334.1 was used as the gL polypeptide, and the native signal peptide (amino acids 1-22 of the NCBI sequence) was replaced with an IgG κ leader sequence. For some of the constructs, amino acids 19-678 of the gH amino acid sequence in NC_009334.1 was used as the gH polypeptide. In some embodiments, the gL and gH were linked via a linker as shown in the table of sequences herein. In some embodiments, the linker comprises an amino acid sequence having at least 80%, at least 85%, at least 90%, at least 95%, at least 96%, at least 97%, at least 98%, at least 99%, or 100% identity to any one of SEQ ID Nos: 3-8. In a particular embodiment, a linker separates the EBV gL polypeptide and the EBV gH polypeptide and has at least 80%, at least 85%, at least 90%, at least 95%, at least 96%, at least 97%, at least 98%, at least 99%, or 100% identity to SEQ ID NO: 3.

In some embodiments, the EBV gH polypeptide comprises an amino acid sequence having at least 80% identity to residues 162-823 of any one of SEQ ID Nos: 9-11; optionally wherein the EBV gH polypeptide comprises a leucine at a residue corresponding to position 189 of any one of SEQ ID Nos: 9-11; a glutamic acid at a residue corresponding to position 368 of any one of SEQ ID Nos: 9-11; and a glutamic acid at a residue corresponding to position 429 of any one of SEQ ID Nos: 9-11. In some embodiments, the EBV gL polypeptide comprises an amino acid sequence having at least 80% identity to residues 1-115 of any one of SEQ ID Nos: 9-11; optionally wherein the EBV gL polypeptide comprises an alanine at a residue corresponding to position 36 of any one of SEQ ID Nos: 9-11; a glutamine at a residue corresponding to position 47 of any one of SEQ ID Nos: 9-11; and a glutamine at a residue corresponding to position 105 of any one of SEQ ID Nos: 9-11.

In some embodiments, gL and gH polypeptides are expressed as a single-chain monomer. In some embodiments, the monomer composition comprises or consists of a sequence shown in the Sequence Table and denoted in the description as “monomer”. A single-chain comprising gL and gH polypeptides may be referred to as “gL/gH,” which can be used interchangeably with “gH_gL,” “gL_gH,” or “gL/gH.”

The gL/gH polypeptide can be combined with any of the ferritins discussed herein. For example, in some embodiments, the fusion protein comprises a monomer or trimer gL/gH polypeptide (+/−gp42 and/or gp220) and a ferritin of the present invention, optionally wherein the ferritin comprises or consists of the amino acids of any one of SEQ ID NOs: 23, 24, 42, and 43.

Additionally, in some embodiments, any fusion protein comprising an EBV gL/gH polypeptide and a ferritin of the invention can be present in a composition comprising another polypeptide disclosed herein.

### D. Fusion Proteins Comprising a gp220 Polypeptide and a Ferritin

In some embodiments, a fusion protein disclosed herein can comprise a gp220 polypeptide and a ferritin disclosed herein (such as a ferritin of any one of SEQ ID NOs: 23, 24, 42, and 43, or residues 435-600 of SEQ ID NO: 14). A gp220-hybrid bullfrog/H. pylori ferritin nanoparticle has been previously described in Kanekiyo Cell. 2015 Aug. 27; 162(5):1090-100.

In some embodiments, the gp220 polypeptide comprises an amino acid sequence having at least 80%, at least 85%, at least 90%, at least 95%, at least 96%, at least 97%, at least 98%, at least 99%, or 100% identity to SEQ ID NO: 18. In some embodiments, the EBV gp220 polypeptide comprises an amino acid sequence having at least 80%, at least 85%, at least 90%, at least 95%, at least 96%, at least 97%, at least 98%, at least 99%, or 100% sequence identity to residues 1-425 of SEQ ID NO: 14.

In some embodiments, a mammalian leader sequence (also known as a signal sequence) is N-terminally appended to a gp220 polypeptide. In some embodiments, a mammalian leader sequence results in secretion of a protein when expressed in mammalian cells. In some embodiments, the mammalian leader sequence comprises an amino acid sequence having at least 80%, at least 85%, at least 90%, at least 95%, at least 96%, at least 97%, at least 98%, at least 99%, or 100% identity to SEQ ID NOs: 12 or 13.

The gp220 polypeptide can be combined with any of the ferritins discussed herein. For example, in some embodiments, a composition comprises a single chain fusion protein comprising a gp220 polypeptide and optionally a ferritin and a single chain fusion protein comprising gL/gH and/or gp42 and a ferritin, wherein the ferritin comprises or consists of the amino acids of any one of SEQ ID NOs: 23, 24, 42, and 43. In some embodiments, a fusion protein comprises a gp220 polypeptide disclosed herein and a ferritin, and the ferritin comprises a serine or cysteine (such as a serine) at a residue corresponding to position 110 of SEQ ID NO: 1; optionally wherein the ferritin comprises an amino acid sequence having at least 80%, at least 85%, at least 90%, at least 95%, at least 96%, at least 97%, at least 98%, at least 99%, or 100% identity to residues 435-600 of SEQ ID NO: 14, or wherein the ferritin comprises or consists of any one of SEQ ID NOs: 23, 24, 42, and 43.

Additionally, in some embodiments, any fusion protein comprising a gp220 polypeptide and a ferritin can be present in a composition comprising another fusion protein and/or another polypeptide disclosed herein.

In some embodiments, the fusion protein comprises a sequence having at least 80%, at least 85%, at least 90%, at least 95%, at least 96%, at least 97%, at least 98%, at least 99%, or 100% identity to SEQ ID NO: 14, optionally further comprising a mammalian leader sequence (such as the mammalian leader sequence of SEQ ID NOs: 12 or 13), and optionally lacking the bullfrog ferritin extension sequence (e.g., of SEQ ID NO: 2) linked to the N-terminus of the ferritin. In some embodiments, the fusion protein comprises a sequence having at least 80%, at least 85%, at least 90%, at least 95%, at least 96%, at least 97%, at least 98%, at least 99%, or 100% identity to SEQ ID NO: 35, optionally lacking the bullfrog ferritin extension sequence (e.g., of SEQ ID NO: 2) linked to the N-terminus of the ferritin. In some embodiments, a fusion protein comprising a gp220 polypeptide and a ferritin comprises a ferritin wherein at least one, or all, surface exposed cysteines are replaced with a different amino acid, such as a serine. In some embodiments, the fusion protein comprises a sequence having at least 80%, at least 85%, at least 90%, at least 95%, at least 96%, at least 97%, at least 98%, at least 99%, or 100% identity to SEQ ID NO: 25, 14 or 35, and the ferritin of the fusion protein comprises a serine at a residue corresponding to position 110 of SEQ ID NO: 1 (i.e., a serine at position C544 of SEQ ID NO: 14 or at position C574 of SEQ ID NO: 35). In one embodiment, the fusion protein comprises or consists of the amino acid sequence of SEQ ID NO: 25.

### E. Fusion Proteins Comprising a gp42 Polypeptide and a Ferritin

In some embodiments, a fusion protein disclosed herein can comprise a gp42 polypeptide and a ferritin disclosed herein (such as a ferritin of any one of SEQ ID NOs: 23, 24, 42, and 43). An exemplary gp42 sequence is provided as SEQ ID NO: 15. A further exemplary gp42 sequence, suitable for inclusion in fusions, e.g., with gL and gH polypeptides, is provided as residues 856-1038 of any one of SEQ ID Nos: 9-11.

In some embodiments, the gp42 polypeptide comprises an amino acid sequence with at least 80%, at least 85%, at least 90%, at least 95%, at least 96%, at least 97%, at least 98%, at least 99%, or 100% identity to SEQ ID NO: 15. In some embodiments, the gp42 polypeptide comprises an amino acid sequence with at least 80%, at least 85%, at least 90%, at least 95%, at least 96%, at least 97%, at least 98%, at least 99%, or 100% identity to residues 856-1038 of any one of SEQ ID Nos: 9-11.

In some embodiments, a mammalian leader sequence (also known as a signal sequence) is N-terminally appended to a gp42 polypeptide. In some embodiments, a mammalian leader sequence results in secretion of a protein when expressed in mammalian cells. In some embodiments, the mammalian leader sequence comprises an amino acid sequence with at least 80%, at least 85%, at least 90%, at least 95%, at least 96%, at least 97%, at least 98%, at least 99%, or 100% identity to SEQ ID NOs: 12 or 13.

In some embodiments, a fusion protein comprising a gH and/or gL polypeptide further comprises a gp42 polypeptide. Any of the fusion proteins comprising a gH and/or gL polypeptide described above can further comprise a gp42 polypeptide. In some embodiments, the gp42 polypeptide is located C-terminal to the gH and/or gL polypeptide(s), as exemplified in SEQ ID Nos: 9-11 and 36-41. In some embodiments, the gp42 polypeptide is located N-terminal to a ferritin, also as exemplified in SEQ ID Nos: 9-11 and 36-41. Thus, for example, a fusion protein may comprise, in N- to C-terminal order, a gL polypeptide, a gH polypeptide, a gp42 polypeptide, and a ferritin of the present invention. Stated differently, in some embodiments, an EBV gL polypeptide, an EBV gH polypeptide, an EBV gp42 polypeptide, and a ferritin of the present invention are arranged in N-terminal to C-terminal order within the fusion protein. In some embodiments, the ferritin comprises or consists of the amino acids of any one of SEQ ID NOs: 23, 24, 42, and 43.

Linkers such as those described herein can separate the gp42 polypeptide from one or more EBV polypeptides and/or ferritins located N-terminal and/or C-terminal thereto. In some embodiments, a linker separates each EBV polypeptide in a fusion protein (e.g., a gL polypeptide, a gH polypeptide, and a gp42 polypeptide), and a further linker may be present between the ferritin and the EBV polypeptide (e.g., a gp42 polypeptide).

In some embodiments, a linker separates the EBV gH polypeptide and the EBV gp42 polypeptide and has a length of at least 15 amino acids, such as 15-100 amino acids, such as 15-90, 15-80, 15-70, 15-60, 15-50, 15-40, 15-30, or 15-20 amino acids. Such a linker may have a length of 15 to 60 amino acids, 20 to 60 amino acids, 30 to 60 amino acids, 40 to 60 amino acids, 30 to 50 amino acids, 30 to 40 amino acids, 32 amino acids, or 40 to 50 amino acids. In some embodiments, a linker separating the EBV gH polypeptide and the EBV gp42 polypeptide has at least 80%, at least 85%, at least 90%, at least 95%, at least 96%, at least 97%, at least 98%, at least 99%, or 100% identity to any one of SEQ ID Nos: 3-8. In a particular embodiment, a linker separating the EBV gH polypeptide and the EBV gp42 polypeptide has at least 80%, at least 85%, at least 90%, at least 95%, at least 96%, at least 97%, at least 98%, at least 99%, or 100% identity to SEQ ID NO: 4.

In some embodiments, a linker separates the EBV gp42 polypeptide and the ferritin. Such a linker may have a length of at least 15 amino acids, such as 15-100 amino acids, such as 15-90, 15-80, 15-70, 15-60, 15-50, 15-40, 15-30, or 15-20 amino acids, or has a length of 15 to 60 amino acids, 20 to 60 amino acids, 30 to 60 amino acids, 40 to 60 amino acids, 30 to 50 amino acids, or 40 to 50 amino acids, 70 to 90 amino acids, 80 to 90 amino acids, or 88 amino acids. In some embodiments, such a linker comprises an amino acid sequence having at least 80%, at least 85%, at least 90%, at least 95%, at least 96%, at least 97%, at least 98%, at least 99%, or 100% identity to any one of SEQ ID NOs: 3-8. In particular embodiments, the further linker comprises an amino acid sequence having at least 80%, at least 85%, at least 90%, at least 95%, at least 96%, at least 97%, at least 98%, at least 99%, or 100% identity to SEQ ID NO: 5.

The gp42 polypeptide can be made into a fusion protein with any of the ferritins discussed herein. For example, in some embodiments, a fusion protein comprises a gp42 polypeptide (+/−gL/gH and/or gp220) and ferritin. In some embodiments, the ferritin comprises or consists of the amino acids of any one of SEQ ID NOs: 23, 24, 42, and 43.

In some embodiments, the fusion protein comprises a sequence having at least 80%, at least 85%, at least 90%, at least 95%, at least 96%, at least 97%, at least 98%, at least 99%, or 100% identity to any one of SEQ ID NOs: 9-11, optionally further comprising a mammalian leader sequence (such as the mammalian leader sequence of SEQ ID NOs: 12 or 13, such as shown in SEQ ID NOs: 36-41), and optionally lacking the bullfrog ferritin extension sequence (of SEQ ID NO: 2) linked to the N-terminus of the ferritin. In certain embodiments, the ferritin comprises or consists of the amino acids of any one of SEQ ID NOs: 23, 24, 42, and 43.

Additionally, in some embodiments, any fusion protein comprising a gp42 polypeptide and a ferritin of the invention can be present in a composition comprising another fusion protein and/or polypeptide disclosed herein.

### F. Linkers

In some embodiments, the fusion protein in Sections B or C comprises a linker between gL and gH polypeptides. In some embodiments, the fusion protein in Section B or E comprises a linker between gH and gp42 polypeptides. In some embodiments, the fusion protein in Sections B, C, D or E comprises a linker between an EBV polypeptide and a ferritin of the present invention. The present invention provides that a relatively long linker between the gL and gH sequences may provide an increase in immunogenicity. In some embodiments, the linker is a peptide linker, which can facilitate expression of a fusion protein (e.g., from a single open reading frame). In some embodiments, the linker is a glycine-serine linker. Exemplary linkers are provided in Table 2.

In some embodiments, the linker is at least 15 amino acids in length. In some embodiments, the linker is at least 25 amino acids in length. In some embodiments, the linker is at least 30 amino acids in length. In some embodiments, the linker is at least 35 amino acids in length. In some embodiments, the linker is at least 40 amino acids in length. In some embodiments, the linker is less than or equal to 60 amino acids in length. In some embodiments, the linker is less than or equal to 50 amino acids in length. In some embodiments, the linker is about 16, 28, 40, 46, or 47 amino acids in length. In some embodiments, the linker is flexible. In some embodiments, the linker comprises a cysteine, e.g., for use as a site for conjugation of, e.g., adjuvant; an exemplary linker comprising a cysteine is provided as SEQ ID NO: 29 (SGGGSGSASSGASASGSSCSGSGSGSSSASSGASSGGASGGGSGGSG). In some embodiments, the linker comprises a sequence with at least 80%, at least 85%, at least 90%, at least 95%, at least 96%, at least 97%, at least 98%, at least 99%, or 100% identity to SEQ ID NO: 29, and further comprises a cysteine corresponding to the cysteine in SEQ ID NO: 29. In some embodiments, the linker comprises at least 25 amino acids (e.g., 25 to 60 amino acids), wherein a cysteine is located at a position ranging from the 8^{th} amino acid from the N-terminus to the 8^{th} amino acid from the C-terminus, or within 10 amino acids of the central residue or bond of the linker.

In some embodiments, the linker comprises glycine (G) and/or serine (S) amino acids. In some embodiments, the linker comprises or consists of glycine (G), serine (S), asparagine (N), and/or alanine (A) amino acids, and optionally a cysteine as discussed above. In some embodiments, the linker comprises an amino acid sequence with at least 80%, at least 85%, at least 90%, at least 95%, at least 96%, at least 97%, at least 98%, at least 99%, or 100% identity to GGSGSASSGASASGSSNGSGSGSGSNSSASSGASSGGASGGSGGSG (SEQ ID NO: 30). In some embodiments, the linker comprises GGGGSGGGGSGGGGSG (SEQ ID NO: 31), GGSGSGSNSSASSGASSGGASGGSGGSG (SEQ ID NO: 32), SEQ ID NO: 30, or GS. In some embodiments, the linker comprises FR1 (GGSGSASAEAAAKEAAAKAGGSGGSG; SEQ ID NO: 33) or FR2 (GGSGSASAEAAAKEAAAKEAAAKASGGSGGSG; SEQ ID NO: 34). In some embodiments, the linker comprises or consists of SEQ ID NO: 3, 6 or 8. In some embodiments, the linker comprises an amino acid sequence with at least 80%, at least 85%, at least 90%, at least 95%, at least 96%, at least 97%, at least 98%, at least 99%, or 100% identity to SEQ ID NO: 3. In some embodiments, the linker comprises an amino acid sequence with at least 80%, at least 85%, at least 90%, at least 95%, at least 96%, at least 97%, at least 98%, at least 99%, or 100% identity to SEQ ID NO: 6. In some embodiments, the linker comprises an amino acid sequence with at least 80%, at least 85%, at least 90%, at least 95%, at least 96%, at least 97%, at least 98%, at least 99%, or 100% identity to SEQ ID NO: 8.

In some embodiments, a linker separates the EBV gL polypeptide and the EBV gH polypeptide and has a length of at least 15 amino acids, such as 15-100 amino acids, such as 15-90, 15-80, 15-70, 15-60, 15-50, 15-40, 15-30, or 15-20 amino acids. Such a linker may have a length of 15 to 60 amino acids, 20 to 60 amino acids, 30 to 60 amino acids, 40 to 60 amino acids, 30 to 50 amino acids, 30 to 40 amino acids, 44 to 48 amino acids, 44 amino acids, 46 amino acids, 48 amino acids, or 40 to 50 amino acids. In some embodiments, a linker separating the EBV gL polypeptide and the EBV gH polypeptide has at least 80%, at least 85%, at least 90%, at least 95%, at least 96%, at least 97%, at least 98%, at least 99%, or 100% identity to any one of SEQ ID Nos: 3-8 and 29-30. In a particular embodiment, a linker separating the EBV gL polypeptide and the EBV gH polypeptide has at least 80%, at least 85%, at least 90%, at least 95%, at least 96%, at least 97%, at least 98%, at least 99%, or 100% identity to SEQ ID NO: 3. In a particular embodiment, a linker separating the EBV gL polypeptide and the EBV gH polypeptide comprises or consists of SEQ ID NO: 3.

In some embodiments, a linker separates the EBV gH polypeptide and the EBV gp42 polypeptide and has a length of at least 15 amino acids, such as 15-100 amino acids, such as 15-90, 15-80, 15-70, 15-60, 15-50, 15-40, 15-30, or 15-20 amino acids. Such a linker may have a length of 15 to 60 amino acids, 20 to 60 amino acids, 30 to 60 amino acids, 40 to 60 amino acids, 30 to 50 amino acids, 30 to 40 amino acids, 32 amino acids, or 40 to 50 amino acids. In some embodiments, a linker separating the EBV gH polypeptide and the EBV gp42 polypeptide has at least 80%, at least 85%, at least 90%, at least 95%, at least 96%, at least 97%, at least 98%, at least 99%, or 100% identity to any one of SEQ ID Nos: 3-8. In a particular embodiment, a linker separating the EBV gH polypeptide and the EBV gp42 polypeptide has at least 80%, at least 85%, at least 90%, at least 95%, at least 96%, at least 97%, at least 98%, at least 99%, or 100% identity to SEQ ID NO: 4. In a particular embodiment, a linker separating the EBV gH polypeptide and the EBV gp42 polypeptide comprises or consists of SEQ ID NO: 4.

In some embodiments, a linker separates the EBV gp42 polypeptide and the ferritin. Such a linker may have a length of at least 15 amino acids, such as 15-100 amino acids, such as 15-90, 15-80, 15-70, 15-60, 15-50, 15-40, 15-30, or 15-20 amino acids, or has a length of 15 to 60 amino acids, 20 to 60 amino acids, 30 to 60 amino acids, 40 to 60 amino acids, 30 to 50 amino acids, or 40 to 50 amino acids, 70 to 90 amino acids, 80 to 90 amino acids, or 88 amino acids. In some embodiments, such a linker comprises an amino acid sequence having at least 80%, at least 85%, at least 90%, at least 95%, at least 96%, at least 97%, at least 98%, at least 99%, or 100% identity to any one of SEQ ID NOs: 3-8. In particular embodiments, a linker separating the EBV gp42 polypeptide and the ferritin comprises an amino acid sequence having at least 80%, at least 85%, at least 90%, at least 95%, at least 96%, at least 97%, at least 98%, at least 99%, or 100% identity to SEQ ID NO: 5. In particular embodiments, a linker separating the EBV gp42 polypeptide and the ferritin comprises or consists of SEQ ID NO: 5.

In some embodiments, a fusion protein that comprises gL, gH and gp42 and a ferritin of the invention comprises a linker between gL and gH, gH and gp42, and gp42 and the ferritin. In one embodiment, the linker that separates the EBV gL polypeptide and the EBV gH polypeptide comprises or consists of the amino acid sequence of SEQ ID NO: 3. In one embodiment, the linker that separates the EBV gL polypeptide and the EBV gH polypeptide has 40 to 50 amino acids. In one embodiment, the linker that separates the EBV gL polypeptide and the EBV gH polypeptide has 44, 46, or 47 amino acids. In one embodiment, the linker that separates the EBV gH polypeptide and the EBV gp42 polypeptide comprises or consists of the amino acid sequence of SEQ ID NO: 4. In one embodiment, the linker that separates the EBV gH polypeptide and the EBV gp42 polypeptide has 30 to 40 amino acids. In one embodiment, the linker that separates the EBV gH polypeptide and the EBV gp42 polypeptide has 32 amino acids. In one embodiment, the linker that separates the EBV gp42 polypeptide and the ferritin comprises or consists of the amino acid sequence of SEQ ID NO: 5. In one embodiment, the linker that separates the EBV gp42 polypeptide and the ferritin has 80 to 90 amino acids. In one embodiment, the linker that separates the EBV gp42 polypeptide and the ferritin has 88 amino acids.

In a particular embodiment, the linker between the EBV gL polypeptide and the EBV gH polypeptide is 44 to 48 amino acids, and the linker between the EBV gH polypeptide and the EBV gp42 polypeptide is 32 amino acids. In another particular embodiment, the linker between the EBV gL polypeptide and the EBV gH polypeptide is 46 amino acids, and the linker between the EBV gH polypeptide and the EBV gp42 polypeptide is 32 amino acids. In another particular embodiment, a linker separates the EBV gL polypeptide and the EBV gH polypeptide and consists of an amino acid sequence that has at least 80% identity to SEQ ID NO: 3; and/or a linker separates the EBV gH polypeptide and the EBV gp42 polypeptide and consists of an amino acid sequence that has at least 80% identity to SEQ ID NO: 4.

In some particular embodiments, a fusion protein described above further comprises a further linker that separates the ferritin and the EBV gp42 polypeptide. In particular embodiments, the further linker has a length of 88 amino acids. In particular embodiments, the further linker consists of an amino acid sequence having at least 80% identity to SEQ ID NO: 5.

### G. Mutations in gL, gH, gp42, Linker, and/or Ferritin Sequences to Eliminate Potential Oxidation, Deamidation, or Isoaspartate Formation Sites

In some embodiments, the fusion protein of Sections B, C or E comprises one or more mutations to eliminate potential oxidation, deamidation, or isoaspartate formation sites, such as the exemplary mutations set forth in Table 1 below.

For example, in some embodiments, a gL sequence comprises one or more mutations to eliminate a potential succinimide/isoaspartate or deamidation site. For example, a gL sequence can comprise a G to A mutation at a position corresponding to position 36 of SEQ ID NO: 16; an N to Q mutation at a position corresponding to position 47 of SEQ ID NO: 16; and/or an N to Q mutation at a position corresponding to position 105 of SEQ ID NO: 16. In some embodiments, a gL sequence comprises each of a G to A mutation at a position corresponding to position 36 of SEQ ID NO: 16; an N to Q mutation at a position corresponding to position 47 of SEQ ID NO: 16; and an N to Q mutation at a position corresponding to position 105 of SEQ ID NO: 16. A position in an amino acid sequence “corresponds” to a given position in SEQ ID NO: 16 if it aligns to that position according to a standard sequence alignment algorithm such as the Smith-Waterman algorithm using default parameters.

In some embodiments, a linker comprises one or more mutations to eliminate a potential deamidation site, for example, an N to G mutation.

In some embodiments, a gH sequence comprises one or more mutations to eliminate a potential succinimide/isoaspartate or oxidation site. For example, a gH sequence can comprise one or more of an M to L mutation at a position corresponding to position 28, 240, or 568 of SEQ ID NO: 17; a D to E mutation at a position corresponding to position 207 or 268 of SEQ ID NO: 17; an M to I mutation at a position corresponding to position 338 or 478 of SEQ ID NO: 17; or an N to Q mutation at a position corresponding to position 492 of SEQ ID NO: 17. In some embodiments, a gH sequence comprises each of an M to L mutation at a position corresponding to position 28 of SEQ ID NO: 17, and a D to E mutation at positions corresponding to positions 207 and 268 of SEQ ID NO: 17.

In some embodiments, a gp42 sequence comprises one or more mutations to eliminate a potential deamidation site. For example, a gp42 sequence can comprise one or more of an N to Q mutation at a position corresponding to position 131 or 162 of SEQ ID NO: 15; or an N to S mutation at a position corresponding to position 160 of SEQ ID NO: 15. In some embodiments, a gp42 sequence comprises each of an N to Q mutation at positions corresponding to positions 131 and 162 of SEQ ID NO: 15, and an N to S mutation at a position corresponding to position 160 of SEQ ID NO: 15. In some embodiments, a gp42 sequence does not comprise any of these mutations.

In some embodiments, a ferritin sequence comprises one or more mutations to eliminate a potential deamidation, oxidation, or isoaspartate formation site. For example, a ferritin sequence can an M to I mutation at a position corresponding to position 17 of SEQ ID NO: 1; an M to L mutation at a position corresponding to position 26 of SEQ ID NO: 1; a G to A mutation at a position corresponding to position 37 of SEQ ID NO: 1; or an N to Q mutation at a position corresponding to position 102 or 145 of SEQ ID NO: 1.

Exemplary mutations are shown below in Table 1.

TABLE 1
Exemplary mutations.

| | | | | | | | Reference |
|---|---|---|---|---|---|---|---|
| Location | Modification | START | END | MOTIF | solvent exposure | Mutation | SEQ ID NO |
| gL | Succinimide/IsoAsp | 35 | 36 | DG | Exposed | G36A | 16 |
| gL | deamidation | 47 | 47 | N | likely exposed | N47Q | 16 |
| gL | deamidation | 105 | 105 | N | exposed | N105Q | 16 |
| gH | oxidation | 28 | 28 | M | exposed | M28L | 17 |
| gH | Succinimide/IsoAsp | 207 | 207 | DY | exposed | D207E | 17 |
| gH | oxidation | 240 | 240 | M | buried | M240L | 17 |
| gH | Succinimide/IsoAsp | 268 | 269 | DT | exposed | D268E | 17 |
| gH | oxidation | 338 | 338 | M | exposed | M338I | 17 |
| gH | oxidation | 478 | 478 | M | exposed | M478I | 17 |
| gH | oxidation | 492 | 492 | N | exposed | N492Q | 17 |
| gH | oxidation | 568 | 568 | M | exposed | M568L | 17 |
| gp42 | deamidation | 131 | 131 | N | exposed | N131Q | 15 |
| gp42 | deamidation | 160 | 160 | N | exposed | N160S | 15 |
| gp42 | deamidation | 162 | 162 | N | exposed | N162Q | 15 |
| ferritin | oxidation | 17 | 17 | M | buried | M17I | 1 |
| ferritin | deamidation | 26 | 26 | M | buried | M26L | 1 |
| ferritin | IsoAsp | 36 | 37 | DG | buried | G37A | 1 |
| ferritin | deamidation | 102 | 102 | N | exposed | N102Q | 1 |
| ferritin | deamidation | 145 | 145 | N | exposed | N145Q | 1 |

### H. Exemplary Compositions, Kits, Nucleic Acids, Uses, and Methods

In some embodiments, the present invention provides methods of immunizing or vaccinating a subject against infection with EBV. The present invention further provides methods of eliciting an immune response against EBV in a subject. In some embodiments, the present methods comprise administering to the subject an effective amount of a pharmaceutical composition described herein to a subject. In some embodiments, the present methods comprise administering to the subject an effective amount of a fusion protein or nanoparticle described herein to a subject.

In some embodiments, a composition comprising any one or more of the fusion proteins described herein and a pharmaceutically acceptable vehicle, adjuvant, or excipient is provided.

In some embodiments, a fusion protein, nanoparticle, or composition described herein is administered to a subject, such as a human or any of the subjects discussed below, to immunize against infection caused by EBV. In some embodiments, a fusion protein or nanoparticle described herein is administered to a subject, such as a human, to produce a protective immune response to future infection with EBV. In some embodiments, a fusion protein is administered. In some embodiments, a fusion protein comprising an EBV polypeptide and ferritin is administered, wherein the ferritin can have one or more mutations described herein. In some embodiments, a fusion protein or nanoparticle having at least 80%, at least 85%, at least 90%, at least 95%, at least 96%, at least 97%, at least 98%, at least 99%, or 100% sequence identity to any one of SEQ ID NOS: 9-11, 14, 25, or 36-41 is administered. In some embodiments, a fusion protein or nanoparticle having at least 80%, at least 85%, at least 90%, at least 95%, at least 96%, at least 97%, at least 98%, at least 99%, or 100% sequence identity to any one of SEQ ID NOS: 9-11 or 36-41 and a fusion protein having at least 80%, at least 85%, at least 90%, at least 95%, at least 96%, at least 97%, at least 98%, at least 99%, or 100% sequence identity to SEQ ID NO: 14, 25, or 35 is administered. In some embodiments, a ferritin of the fusion protein or nanoparticle comprises or consists of the amino acids of SEQ ID NO: 23. In some embodiments, a ferritin of the fusion protein or nanoparticle comprises or consists of the amino acids of SEQ ID NO: 24. In some embodiments, a ferritin of the fusion protein or nanoparticle comprises or consists of the amino acids of SEQ ID NO: 42. In some embodiments, a ferritin of the fusion protein or nanoparticle comprises or consists of the amino acids of SEQ ID NO: 43. In some embodiments, a fusion protein or nanoparticle comprising any one of SEQ ID NOS: 9-11 or 36-41 is administered. In some embodiments, a fusion protein or nanoparticle comprising any one of SEQ ID NOS: 9-11 or 36-41 and a fusion protein comprising SEQ ID NO: 14, 25, or 35 is administered.

In some embodiments, the protective immune response decreases the incidence of hospitalization. In some embodiments, the protective immune response decreases the incidence of EBV infection, mononucleosis, complications caused by mononucleosis (e.g., hepatitis, encephalitis, severe hemolytic anemia, or splenomegaly), nasopharyngeal cancer, gastric cancer, or B lymphoma (e.g., Burkitt's or Hodgkin's lymphoma), and multiple sclerosis.

In some embodiments, a composition comprises one fusion protein. In some embodiments, a composition comprises a fusion protein comprising a gH polypeptide and a ferritin of the present invention. In some embodiments, a composition comprises a fusion protein comprising a gL polypeptide and a ferritin of the present invention. In some embodiments, a composition comprises a fusion protein comprising a gp42 polypeptide and a ferritin of the present invention. In some embodiments, a composition comprises a fusion protein comprising a gp220 polypeptide, and optionally a ferritin.

In some embodiments, a composition comprises more than one fusion protein. In some embodiments, a composition comprises one or more fusion proteins comprising more than one EBV polypeptide and at least one ferritin of the present invention. In some embodiments, an EBV vaccine comprises nanoparticles comprising a gp220 polypeptide and optionally a ferritin and, separately, nanoparticles comprising gH and gL polypeptides, and optionally gp42 polypeptides, and a ferritin of the present invention.

In some embodiments, any one or more of the fusion proteins, nanoparticles, or compositions described herein are provided for use in immunizing against infection caused by EBV. In some embodiments, any one or more of the fusion proteins, nanoparticles, or compositions described herein, such as in Sections B, C, D or E, are provided for use in producing a protective immune response to future infection with EBV.

In some embodiments, a composition of the present disclosure comprises a first ferritin nanoparticle comprising a fusion protein disclosed herein (such as a fusion protein comprising an amino acid sequence having at least 80%, at least 85%, at least 90%, at least 95%, at least 96%, at least 97%, at least 98%, at least 99%, or 100% identity to any one of SEQ ID NOs: 9-11, 14, 25, or 35-41) and optionally a second ferritin nanoparticle comprising a second fusion protein comprising an EBV gp220 polypeptide and a ferritin (such as a fusion protein comprising an amino acid sequence having at least 80%, at least 85%, at least 90%, at least 95%, at least 96%, at least 97%, at least 98%, at least 99%, or 100% identity to any one of SEQ ID NOs: 14, 25, or 35). In some embodiments, the EBV gp220 polypeptide of the composition comprises an amino acid sequence having at least 80%, at least 85%, at least 90%, at least 95%, at least 96%, at least 97%, at least 98%, at least 99%, or 100% sequence identity to residues 1-425 of SEQ ID NO: 14. In some embodiments, the ferritin of the second fusion protein comprises a serine or cysteine at a residue corresponding to position 110 of SEQ ID NO: 1; optionally wherein the ferritin comprises an amino acid sequence having at least 80% identity to residues 435-600 of SEQ ID NO:14. In some embodiments, the second fusion protein comprises an amino acid sequence having at least 80%, at least 85%, at least 90%, at least 95%, at least 96%, at least 97%, at least 98%, at least 99%, or 100% sequence identity to SEQ ID NO: 14, optionally wherein position C544 of SEQ ID NO: 14 is substituted to serine.

In some embodiments, a composition of the present disclosure comprises a first ferritin nanoparticle comprising a fusion protein consisting of the amino acid sequence of any one of SEQ ID NOs: 9-11 and (a) a second ferritin nanoparticle comprising a second fusion protein consisting of the amino acid sequence of SEQ ID NO: 14, or (b) a second ferritin nanoparticle comprising a second fusion protein consisting of the amino acid sequence of SEQ ID NO: 14 with a substitution to a serine at position C544 of SEQ ID NO: 14.

In a particular embodiment, a composition of the present disclosure comprises a first ferritin nanoparticle comprising a fusion protein consisting of the amino acid sequence of SEQ ID NO: 11 and (a) a second ferritin nanoparticle comprising a second fusion protein consisting of the amino acid sequence of SEQ ID NO: 14.

### 1. Subjects

In some embodiments, the subject is a mammal. In some embodiments, the subject is a human.

In some embodiments, the subject is an adult (greater than or equal to 18 years of age). In some embodiments, the subject is a child or adolescent (less than 18 years of age). In some embodiments, the subject is elderly (greater than 60 years of age). In some embodiments, the subject is a non-elderly adult (greater than or equal to 18 years of age and less than or equal to 60 years of age).

In some embodiments, the composition is suitably formulated for an intended route of administration. Examples of suitable routes of administration include intramuscular, transcutaneous, subcutaneous, intranasal, oral, or transdermal.

In some embodiments, more than one administration of the composition is administered to the subject. In some embodiments, a booster administration improves the immune response.

In some embodiments, any one or more of the fusion proteins or compositions described herein are for use in a mammal, such as a primate (e.g., non-human primate, such as a monkey (e.g., a macaque, such as rhesus or cynomolgus) or ape), rodent (e.g., mouse or rat), or domesticated mammal (e.g., dog, rabbit, cat, horse, sheep, cow, goat, camel, or donkey).

### 2. Adjuvants

An adjuvant may be administered together with the fusion proteins and/or nanoparticles described herein to a subject, wherein administration of such a combination may produce a higher titer of antibodies against the fusion protein(s) in the subject as compared to administration of the fusion protein(s) without the adjuvant. An adjuvant may promote earlier, more potent, or more persistent immune response to the fusion protein(s).

In some embodiments, a composition comprises one adjuvant. In some embodiments, a composition comprises more than one adjuvant. In some embodiments, a composition does not comprise an adjuvant.

In some embodiments, an adjuvant comprises aluminum. In some embodiments, an adjuvant is aluminum phosphate. In some embodiments, an adjuvant is Alum (Alyhydrogel '85 2%; Brenntag—Cat #21645-51-2).

In some embodiments, an adjuvant is an organic adjuvant. In some embodiments, an adjuvant is an oil-based adjuvant. In some embodiments, an adjuvant comprises an oil-in-water nanoemulsion.

In some embodiments, an adjuvant comprises squalene. In some embodiments, the adjuvant comprising squalene is Ribi (Sigma adjuvant system Cat #S6322-lvl), Addavax™ MF59, AS03, or AF03 (see U.S. Pat. No. 9,703,095). In some embodiments, the adjuvant comprising squalene is a nanoemulsion.

In some embodiments, an adjuvant comprises a polyacrylic acid polymer (PAA). In some embodiments, the adjuvant comprising PAA is SPA09 (see WO 2017218819).

In some embodiments, an adjuvant comprises non-metabolizable oils. In some embodiments, the adjuvant is Incomplete Freund's Adjuvant (IFA).

In some embodiments, an adjuvant comprises non-metabolizable oils and killed Mycobacterium tuberculosis. In some embodiments, the adjuvant is Complete Freund's Adjuvant (CFA).

In some embodiments, an adjuvant is a lipopolysaccharide. In some embodiments, an adjuvant is monophosphoryl A (MPL or MPLA).

### 3. Pharmaceutical Compositions

In various embodiments, a pharmaceutical composition comprising a fusion protein described herein and/or related entities is provided. In some embodiments, the pharmaceutical composition is an immunogenic composition (e.g., a vaccine) capable of eliciting an immune response such as a protective immune response against a pathogen.

For example, in some embodiments, the pharmaceutical compositions may comprise one or more of the following: (1) a fusion protein comprising (a) an Epstein Barr virus (EBV) polypeptide comprising: (i) an EBV gH polypeptide and/or (ii) an EBV gL polypeptide; and (b) a ferritin of SEQ ID NO: 1, comprising at least one of the following substitutions: (i) a glutamine at a residue corresponding to position N102 of SEQ ID NO: 1; (ii) a glutamine at a residue corresponding to position N145 of SEQ ID NO: 1; or (iii) a combination thereof, (2) a fusion protein comprising an Epstein Barr virus (EBV) polypeptide comprising: (i) an EBV gH polypeptide and/or (ii) an EBV gL polypeptide; and a ferritin comprising the amino acid sequence of SEQ ID NO: 1 with at least one of the following amino acid substitutions: (i) a glutamine at a residue corresponding to position N102 of SEQ ID NO: 1; (ii) a glutamine at a residue corresponding to position N145 of SEQ ID NO: 1; and/or (iii) a bullfrog ferritin extension sequence that is linked at the N-terminus of the ferritin, wherein the bullfrog ferritin extension sequence comprises the amino acid sequence of SEQ ID NO: 2; (3) a fusion protein comprising an Epstein Barr virus (EBV) polypeptide comprising: (i) an EBV gp42 polypeptide and (ii) an EBV gH polypeptide and/or an EBV gL polypeptide; and a ferritin comprising the amino acid sequence of SEQ ID NO: 1 with at least one of the following amino acid substitutions: (i) a glutamine at a residue corresponding to position 102 of SEQ ID NO: 1; (ii) a glutamine at a residue corresponding to position 145 of SEQ ID NO: 1; and/or (iii) a bullfrog ferritin extension sequence that is linked at the N-terminus of the ferritin, wherein the bullfrog ferritin extension sequence comprises the amino acid sequence of SEQ ID NO: 2; (4) a fusion protein comprising an Epstein Barr virus (EBV) polypeptide comprising an EBV gL polypeptide, and an EBV gH polypeptide, wherein the EBV gL polypeptide comprises the sequence of residues 1-115 of SEQ ID NO: 11 and the EBV gH polypeptide comprises the sequence of residues 162-823 of SEQ ID NO: 11; a ferritin polypeptide of SEQ ID NO: 1, or a ferritin polypeptide having SEQ ID NO: 1 and at least one mutation selected from any one or more of a glutamine at a residue corresponding to position N18 of SEQ ID NO: 1; a serine at a residue corresponding to position C30 of SEQ ID NO: 1; glutamine at a residue corresponding to position N102 of SEQ ID NO: 1; a glutamine at a residue corresponding to position N145 of SEQ ID NO: 1; and optionally a bullfrog ferritin extension sequence that is linked at the N-terminus of the ferritin, wherein the bullfrog ferritin extension sequence comprises the amino acid sequence of SEQ ID NO: 2, and further optionally a mammalian leader sequence positioned at the N-terminus of the fusion protein, wherein the mammalian leader sequence comprises the amino acid sequence of SEQ ID NO: 12 or 13; wherein the EBV gL polypeptide, the EBV gH polypeptide, and the ferritin are arranged in N-terminal to C-terminal order within the EBV polypeptide, and the optional mammalian leader sequence is positioned N-terminal to the EBV gL polypeptide; (5) a fusion protein comprising an Epstein Barr virus (EBV) polypeptide comprising an EBV gL polypeptide, an EBV gH polypeptide, and an EBV gp42 polypeptide, wherein the EBV gL polypeptide comprises the amino acid sequence of residues 1-115 of SEQ ID NO: 11, the EBV gH polypeptide comprises the amino acid sequence of residues 162-823 of SEQ ID NO: 11, and the EBV gp42 polypeptide comprises the amino acid sequence of residues 856-1038 of SEQ ID NO: 11; a ferritin comprising SEQ ID NO: 24; and optionally a mammalian leader sequence positioned at the N-terminus of the fusion protein, wherein the mammalian leader sequence comprises the amino acid sequence of SEQ ID NO: 12 or 13; wherein the EBV gL polypeptide, the EBV gH polypeptide, the EBV gp42 polypeptide, and the ferritin are arranged in N-terminal to C-terminal order within the EBV polypeptide, and the optional mammalian leader sequence is positioned N-terminal to the EBV gL polypeptide; or (6) a ferritin particle comprising any of the foregoing polypeptides. In some embodiments, the pharmaceutical compositions may comprise fusion protein comprising an antigenic EBV gL/gH polypeptide, a fusion protein comprising an antigenic EBV gL/gH/gp42 polypeptide, and/or a fusion protein comprising an antigenic EBV pg220 polypeptide, e.g., wherein the fusion protein comprises a linker of at least 15 amino acids between the gL and gH polypeptide sequences, and/or between the gH and gp42 sequences, and optionally wherein the fusion protein further comprises a ferritin. In some embodiments, the ferritin comprises or consists of the amino acids of any one of SEQ ID NOs: 23, 24, 42, and 43.

In some embodiments, any one of the pharmaceutical compositions described herein is administered alone or in combination with one or more agents to enhance an immune response, e.g., an adjuvant described above. In some embodiments, a pharmaceutical composition further comprises an adjuvant described above.

In some embodiments, the pharmaceutical composition further comprises a pharmaceutically acceptable carrier or excipient. As used herein, the term “carrier” refers to a diluent, adjuvant, excipient, or vehicle with which a pharmaceutical composition is administered. In exemplary embodiments, carriers can include sterile liquids, such as, for example, water and oils, including oils of petroleum, animal, vegetable, or synthetic origin, such as, for example, peanut oil, soybean oil, mineral oil, sesame oil, and the like. In some embodiments, carriers are or include one or more solid components. Pharmaceutically acceptable carriers can also include, but are not limited to, saline, buffered saline, dextrose, glycerol, ethanol, and combinations thereof. As used herein, an excipient is any non-therapeutic agent that may be included in a pharmaceutical composition, for example, to provide or contribute to a desired consistency or stabilizing effect. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In various embodiments, the pharmaceutical composition is sterile.

In some embodiments, the pharmaceutical composition contains minor amounts of wetting or emulsifying agents, or pH buffering agents. In some embodiments, the pharmaceutical compositions may include any of a variety of additives, such as stabilizers, buffers, or preservatives. In addition, auxiliary, stabilizing, thickening, lubricating, and coloring agents can be included.

The described pharmaceutical composition may be administered in any dose appropriate to achieve a desired outcome. In some embodiments, the desired outcome is the induction of a long-lasting adaptive immune response against a pathogen, due to a non-ferritin polypeptide present in a fusion protein in the composition. In some embodiments, the desired outcome is a reduction in the intensity, severity, frequency, and/or delay of onset of one or more symptoms of infection. In some embodiments, the desired outcome is the inhibition or prevention of infection. The dose required will vary from subject to subject depending on the species, age, weight, and general condition of the subject, the severity of the infection being prevented or treated, the particular composition being used, and its mode of administration.

In some embodiments, pharmaceutical compositions in accordance with the invention are administered in single or multiple doses. In some embodiments, the pharmaceutical compositions are administered in multiple doses administered on different days (e.g., prime-boost vaccination strategies). In some embodiments, the pharmaceutical composition is administered as part of a booster regimen.

In various embodiments, the pharmaceutical composition is co-administered with one or more additional therapeutic agents. Co-administration does not require the therapeutic agents to be administered simultaneously, if the timing of their administration is such that the pharmacological activities of the additional therapeutic agent and the active ingredient(s) in the pharmaceutical composition overlap in time, thereby exerting a combined therapeutic effect. In general, each agent will be administered at a dose and on a time schedule determined for that agent.

### 4. Nucleic Acid/mRNA

Also provided is a nucleic acid encoding a fusion protein described herein and any one of Sections B-E. In some embodiments, a nucleic acid of use herein encodes a fusion protein comprising the amino acid sequence of any one of SEQ ID NOs: 9-11, 14, 25, or 35-41. In some embodiments, a nucleic acid of use herein encodes a fusion protein consisting of the amino acid sequence of any one of SEQ ID NOs: 9-11, 14, 25, or 35-41. In some embodiments, a nucleic acid of use herein encodes a ferritin comprising or consisting of the amino acids of any one of SEQ ID NOs: 23, 24, 42, and 43. In some embodiments, the nucleic acid is an mRNA. Any nucleic acid capable of undergoing translation resulting in a polypeptide is considered an mRNA for purposes of this disclosure. In some embodiments, the nucleic acid comprises a sequence having at least 80%, at least 85%, at least 90%, at least 95%, at least 96%, at least 97%, at least 98%, at least 99%, or 100% sequence identity to any one of SEQ ID NOs: 19-22. In some embodiments, the nucleic acid comprises or consists of the sequence of any one of SEQ ID NOs: 19-22.

### 5. Expression Vectors

Also provided herein are expression vectors and cells comprising (such as encoding) a fusion protein described herein. Vectors of use herein can be any suitable recipient nucleic acid molecule modified to comprise or incorporate a provided nucleic acid sequence, such as a nucleic acid sequence encoding a fusion protein disclosed herein. Such suitable vectors include, but are not limited to, nucleic acid vectors (such as plasmids), viral vectors (such as AAV vectors), bacterial vectors, and/or yeast vectors as appropriate. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome.

Vectors may include at least one restriction endonuclease recognition sequence, e.g., for insertion of a transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules, and can be linear or circular nucleic acid molecules. A donor nucleic acid used for gene editing methods employing zinc finger nuclease, TALEN, or CRISPR/Cas can be a type of vector. One type of vector is a plasmid, which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral, and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. A vector may be a transfer vector, i.e., a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

A vector may be (alternatively or additionally) an expression vector, i.e., a vector comprising a recombinant polynucleotide comprising expression control sequences operatively linked to a nucleotide sequence to be expressed. Such expression control sequences may direct expression of inserted genes to which they are operatively linked. An expression vector can comprise sufficient cis-acting elements for expression; other elements for expression can be supplied by the host cell or in an in vitro expression system. Further examples of regulatory sequences and other elements for expression are described in, for example, Goeddel, 1990, Gene Expression Technology: Methods in Enzymology 185, Academic Press, San Diego, Calif. and Baron et al., 1995, Nucleic Acids Res. 23:3605-3606. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference for any purpose.

A vector of use herein may comprise a selection marker, such as any suitable selection marker known in the art. Examples of suitable selectable markers for mammalian cells include dihydrofolate reductase (DHFR). Transfected host cells can be placed under selection pressure wherein only the transformants are uniquely adapted to survive by virtue of the selectable gene present in the vector. Selection pressure is imposed by culturing the transfected cells under conditions in which the concentration of selection agent in the medium is successively increased, thereby leading to the amplification of both the selectable gene and the DNA that encodes another nucleic acid sequence of interest (such as a fusion protein disclosed herein). Accordingly, a host cell of use herein may be dihydrofolate reductase (DHFR) deficient, and the expression vector may comprise a polynucleotide sequence that encodes DHFR. In such embodiments, recombinant cells can be cultured in the presence of a DHFR-selection agent, such as methotrexate, to select for cells that have been successfully transfected and express the nucleic acid sequence of interest (such as a fusion protein disclosed herein).

In some embodiments, an expression vector of use herein comprises a nucleic acid disclosed herein, such as a nucleic acid encoding a fusion protein having at least 80%, at least 85%, at least 90%, at least 95%, at least 96%, at least 97%, at least 98%, at least 99%, or 100% sequence identity to any one of SEQ ID NOs: 9-11, 14, 25, or 35-41, and wherein the ferritin of the fusion protein comprises or consists of the amino acids of any one of SEQ ID NOs: 23, 24, 42, and 43. In some embodiments, the expression vector comprises a nucleic acid comprising a sequence having at least 80%, at least 85%, at least 90%, at least 95%, at least 96%, at least 97%, at least 98%, at least 99%, or 100% sequence identity to any one of SEQ ID NOs: 19-21. In some embodiments, the expression vector comprises a nucleic acid comprising the sequence of any one of SEQ ID NOs: 19-21. In some embodiments, the expression vector comprises a nucleic acid consisting of the sequence of any one of SEQ ID NOs: 19-21.

In some embodiments, an expression vector of use herein comprises a nucleic acid encoding a second fusion protein as disclosed herein, such as a nucleic acid encoding a second fusion protein comprising an EBV gp220 polypeptide, and optionally a ferritin, such as a second fusion protein having at least 80%, at least 85%, at least 90%, at least 95%, at least 96%, at least 97%, at least 98%, at least 99%, or 100% sequence identity to SEQ ID NO: 14, 25, or 35. In some embodiments, a ferritin of the second fusion protein comprises or consists of the amino acids of any one of SEQ ID NOs: 23, 24, 42, and 43. In some embodiments, the expression vector comprises a nucleic acid comprising a sequence having at least 80%, at least 85%, at least 90%, at least 95%, at least 96%, at least 97%, at least 98%, at least 99%, or 100% sequence identity to SEQ ID NO: 22. In some embodiments, the expression vector comprises a nucleic acid comprising the sequence of SEQ ID NO: 22. In some embodiments, the expression vector comprises a nucleic acid consisting of the sequence of SEQ ID NO: 22. Such a second fusion protein may be encoded on the same or on a different expression vector as a fusion protein disclosed herein.

### 6. Cells, Methods of Preparing Cells

Also provided herein are cells (such as recombinant cells) comprising a nucleic acid as disclosed herein, and methods of preparing such cells. Host cells for use herein include any suitable cell that may be used for the purpose of producing a recombinant protein (such as a fusion protein disclosed herein) encoded by an expression vector (such as an expression vector disclosed herein) or propagating the expression vector introduced into the host cell. Recombinant cells comprise a heterologous expression vector (such as an expression vector disclosed herein), which may or may not be integrated into the host cell chromosome. In some embodiments, a host cell has been transfected with an expression vector comprising a polynucleotide sequence that encodes a fusion protein disclosed herein, and such a recombinant cell is capable of producing a fusion protein as disclosed herein.

A host cell useful in the present disclosure can be a eukaryotic cell, a fungal cell, an insect cell, a prokaryotic cell (e.g., bacterial or archaeal cell), or a cell from a multicellular organism (e.g., a cell line) cultured as a unicellular entity. In some embodiments, the cell can be a eukaryotic cell, such as a 293T cell (such as a 293 expi cell or a HEK293T cell). Exemplary mammalian cells include without limitation, Chinese hamster ovary (CHO) cells, CHO-K1 cells, CHO-derived cells, human embryonic kidney (HEK) cells (such as HEK293 cells, which express functional adenoviral E1, and HEK-derived cells such as HEK293T or HEK293F), Madin-Darby canine kidney (MDCK) cells, Vero cells, EB66 cells, chicken embryo cells, RK cells, RAF cells, PK15 cells, MRC-5 cells, A549 cells, WEHI cells, 3T3 cells, 10T1/2 cells, BHK cells, COS 1 cells, COS 7 cells, BSC 1 cells, BSC 40 cells, BMT 10 cells, WI38 cells, HeLa cells, Saos cells, C2C12 cells, L cells, HT1080 cells, HepG2 cells, NS-1 cells, and primary fibroblast, hepatocyte, and myoblast cells derived from mammals including human, monkey, mouse, rat, rabbit, and hamster. The selection of the species (e.g., mammalian species) providing the host cells is not a limitation of this invention; nor is a type of mammalian cell, i.e., fibroblast, hepatocyte, tumor cell, etc. It will be understood by the skilled person that the conditions used in the methods disclosed herein will be dependent upon the host cell used. Typical conditions, for example, the culture medium or temperature to be used, are well known in the art. In one embodiment, culturing is performed by incubating a mammalian packaging cell line (e.g., HEK293 or CHO cells) under humidified conditions. In a particular embodiment, the humidified conditions comprise incubating the transfected cells at about 37° C. and about 5% CO₂. In some embodiments, the cell culture comprises a culture medium suitable for culturing CHO cells, such as a chemically defined CHO cell culture medium. In some embodiments, the cell culture is serum-free. Exemplary commercially available cell culture media include, but are not limited to, Dulbecco's Modified Eagle Media (e.g., including serum, such as 10% fetal bovine serum) (Gibco), BalanCD medium (e.g., BalanCD CHO medium; Fujifilm and Irvine Scientific), CD CHO AGT medium (Gibco), or ExpiCHO™ Stable Production Medium (Gibco).

A cell may be transfected, e.g., with one or more (such as one, two, three, or four) expression vectors (such as one, two, three, four, or more expression vectors as disclosed herein) or other nucleic acids through any process known in the art. Transfection methods have been described in the art and include, for example, calcium phosphate co-precipitation, direct micro-injection into cultured cells, electroporation, liposome mediated gene transfer, lipid-mediated transfection, or nucleic acid delivery using high-velocity microprojectiles. Other suitable transfection media include strontium phosphate, polycationic polymers, e.g., Superfect™ (Qiagen), liposomes, and cationic polymers such as polyethylenimine (PEI). In the disclosed embodiments, the term transfection encompasses any means of introducing a nucleic acid inside a host cell, including, but not limited to, transduction or infection with a DNA or RNA virus or viral vector. The resulting cell can be transiently transfected with the exogenous nucleic acid molecule, i.e., the exogenous DNA will not be integrated into the genome of a transfected cell, but rather can exist episomally. Alternatively, the resulting cell can be stably transfected, i.e., the nucleic acid molecule becomes covalently linked with the host cell genome or is maintained and replicated as an episomal unit that can be passed on to progeny cells (e.g., is capable of extra-chromosomal replication at a sufficient rate).

In some embodiments, a cell (such as a recombinant cell) comprises any nucleic acid disclosed herein, and/or any expression vector disclosed herein. In some embodiments, a cell (such as a recombinant cell) expresses a fusion protein as disclosed herein, and optionally a second fusion protein. In some embodiments, a recombinant cell expresses a fusion protein having at least 80%, at least 85%, at least 90%, at least 95%, at least 96%, at least 97%, at least 98%, at least 99%, or 100% sequence identity to any one of SEQ ID NOs: 9-11, 14, 25, or 35-41, wherein a ferritin of the fusion protein comprises or consists of the amino acids of any one of SEQ ID NOs: 23, 24, 42, and 43. In some embodiments, a recombinant cell further expresses a second fusion protein having at least 80%, at least 85%, at least 90%, at least 95%, at least 96%, at least 97%, at least 98%, at least 99%, or 100% sequence identity to SEQ ID NO: 14, 25, or 35, wherein a ferritin of the fusion protein comprises or consists of the amino acids of any one of SEQ ID NOs: 23, 24, 42, and 43.

Also provided herein are methods of preparing a recombinant cell that expresses a fusion protein as disclosed herein, and optionally a second fusion protein as disclosed herein. In some embodiments, a method of preparing such a cell comprises transfecting a population of host cells with one or more (such as one, two, three, four, or more) expression vectors comprising a polynucleotide sequence that encodes a disclosed fusion protein and optionally a second fusion protein; culturing the transfected host cells; and obtaining a recombinant cell that expresses the fusion protein. In a particular embodiment, the host cells are CHO cells. In another particular embodiment, the host cells (such as the CHO cells) are DHFR deficient. In some such embodiments, the expression vector further comprises a polynucleotide sequence that encodes DHFR. In some such embodiments, the host cells (such as the CHO cells) may be cultured in the presence of a DHFR-selection agent, optionally wherein the DHFR-selection agent comprises methotrexate.

Also provided herein are methods of preparing a disclosed fusion protein, such as a fusion protein comprising an amino acid sequence having at least 80%, at least 85%, at least 90%, at least 95%, at least 96%, at least 97%, at least 98%, at least 99%, or 100% identity to any one of SEQ ID NOs: 9-11, 14, 25, or 35-41. Further provided are fusion proteins obtainable or produced by a disclosed method. In some embodiments, a method of preparing a disclosed fusion protein comprises culturing a host cell as disclosed herein (such as a host cell comprising a nucleic acid encoding a disclosed fusion protein, or a host cell comprising an expression vector comprising a nucleic acid encoding a disclosed fusion protein; and optionally further comprising a nucleic acid encoding a second fusion protein as disclosed herein) under conditions whereby the fusion protein (and optionally the second fusion protein) is expressed in the host cell, and recovering the fusion protein (and optionally the second fusion protein) from the cultured host cell culture. In some embodiments, the host cells are CHO cells. In some embodiments, the host cells are DHFR deficient; and the expression vector further comprises a polynucleotide sequence that encodes DHFR. Thus, some embodiments of the disclosed methods comprise culturing the host cells in the presence of a DHFR-selection agent. In a particular embodiment, the DHFR-selection agent comprises methotrexate. In a particular embodiment, the expression vector comprises a polynucleotide sequence that encodes the amino acid sequence of any one of SEQ ID NOs: 9-11 or 36-41. In another particular embodiment, the expression vector comprises a second polynucleotide sequence that encodes the amino acid sequence of any one of SEQ ID NOs: 14, 25, or 35. In a particular embodiment, the expression vector comprises the polynucleotide sequence of any one of SEQ ID NOs: 19-22.

### 7. Kits

Also provided herein are kits comprising one or more fusion proteins, nucleic acids, antigenic ferritin particles, compositions, or pharmaceutical compositions described herein. In some embodiments, a kit further comprises one or more of a solvent, solution, buffer, instructions, or desiccant.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed considering the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items. The term “or” is used in the inclusive sense, i.e., equivalent to “and/or,” unless the context dictates otherwise.

TABLE 2
(SEQUENCE TABLE): DESCRIPTION OF THE SEQUENCES

| | | SEQ ID |
|---|---|---|
| Description | Sequences | NO |
| Exemplary H. | FSKDIEKLLNEQVNKEMNSSNLYMSMSSWCYTHSLDGAGLFLFDHAAEEYEHAKKLIIFLNENNVPVQLTSISAPEHKFE | 1 |
| pylori | GLTQIFQKAYEHEQHISESINNIVDHAIKSKDHATFNFLQWYVAEQHEEEVLFKDILDKIELIGNENHGLYLADQYVKGI | |
| ferritin | AKSRKS | |
| Exemplary | ESQVRSQ | 2 |
| bullfrog | | |
| ferritin | | |
| extension | | |
| sequence | | |
| 46 amino acid | GGSGSASSGASASGSSGGSGSGSGSGSSASSGASSGGASGGSGGSG | 3 |
| linker | | |
| 32 amino acid | SGGGSGSASSGASASGSSGSGSGSGSSSASSG | 4 |
| linker | | |
| 88 amino acid | GGSGSASSGASASGSSGSGSGSGSSSASSGASSGGASGGSGGSGGGSGSASSGASASGSSGSGSGSGSSSASSGASSGGA | 5 |
| linker | SGGSGGSG | |
| 44 amino acid | GGSGSASSGASASGSSGSGSGSGSSSASSGASSGGASGGSGGSG | 6 |
| linker | | |
| 12 amino acid | EPEPEPEPEPGG | 7 |
| rigid linker | | |
| 48 amino acid | SGEPEPEPEPEPEPEPEPEPEPEPEPEPEPEPEPEPEPEPEPEPEPEP | 8 |
| rigid linker | | |
| C16 gL/gH/gp42/ ferritin | 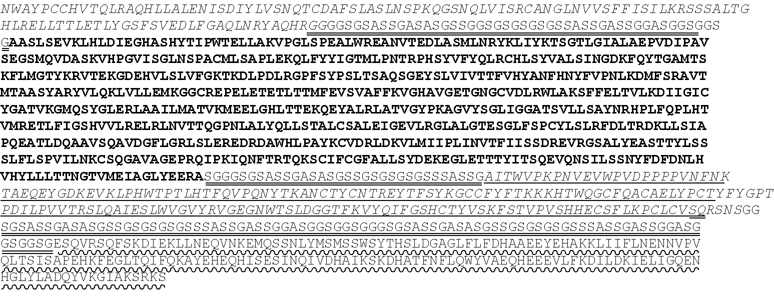 | 9 |
| C17 | NWAYPCCHVTQLRAQHLLALENISDIYLVSNQTCDAFSLASLNSPKQGSNQLVISRCANGLNVVSFFISILKRSSSALTG | 10 |
| gL/gH/gp42/ | HLRELLTTLETLYGSFSVEDLFGAQLNRYAQHRGGGGSGSASSGASASGSSGGSGSGSGSGSSASSGASSGGASGGSGGS | |
| ferritin | GAASLSEVKLHLDIEGHASHYTIPWTELLAKVPGLSPEALWREANVTEDLASMLNRYKLIYKTSGTLGIALAEPVDIPAV | |
| | SEGSMQVDASKVHPGVISGLNSPACMLSAPLEKQLFYYIGTMLPNTRPHSYVFYQLRCHLSYVALSINGDKFQYTGAMTS | |
| | KFLMGTYKRVTEKGDEHVLSLVFGKTKDLPDLRGPFSYPSLTSAQSGEYSLVIVTTFVHYANFHNYFVPNLKDMFSRAVT | |
| | MTAASYARYVLQKLVLLEMKGGCREPELETETLTTMFEVSVAFFKVGHAVGETGNGCVDLRWLAKSFFELTVLKDIIGIC | |
| | YGATVKGMQSYGLERLAAILMATVKMEELGHLTTEKQEYALRLATVGYPKAGVYSGLIGGATSVLLSAYNRHPLFQPLHT | |
| | VMRETLFIGSHVVLRELRLNVTTQGPNLALYQLLSTALCSALEIGEVLRGLALGTESGLFSPCYLSLRFDLTRDKLLSIA | |
| | PQEATLDQAAVSQAVDGFLGRLSLEREDRDAWHLPAYKCVDRLDKVLMIIPLINVTFIISSDREVRGSALYEASTTYLSS | |
| | SLFLSPVILNKCSQGAVAGEPRQIPKIQNFTRTQKSCIFCGFALLSYDEKEGLETTTYITSQEVQNSILSSNYFDFDNLH | |
| | VHYLLLTTNGTVMEIAGLYEERASGGGSGSASSGASASGSSGSGSGSGSSSASSGAITWVPKPNVEVWPVDPPPPVNFNK | |
| | TAEQEYGDKEVKLPHWTPTLHTFQVPQNYTKANCTYCNTREYTFSYKGCCFYFTKKKHTWQGCFQACAELYPCTYFYGPT | |
| | PDILPVVTRSLQAIESLWVGVYRVGEGNWTSLDGGTFKVYQIFGSHCTYVSKFSTVPVSHHECSFLKPCLCVSQRSNSGG | |
| | SGSASSGASASGSSGSGSGSGSSSASSGASSGGASGGSGGSGGGSGSASSGASASGSSGSGSGSGSSSASSGASSGGASG | |
| | GSGGSGESQVRSQFSKDIEKLLNEQVNKEMQSSNLYMSMSSWSYTHSLDGAGLFLFDHAAEEYEHAKKLIIFLNENNVPV | |
| | QLTSISAPEHKFEGLTQIFQKAYEHEQHISESINQIVDHAIKSKDHATFNFLQWYVAEQHEEEVLFKDILDKIELIGQEN | |
| | HGLYLADQYVKGIAKSRKS | |
| C18 gL/gH/gp42/ ferritin | 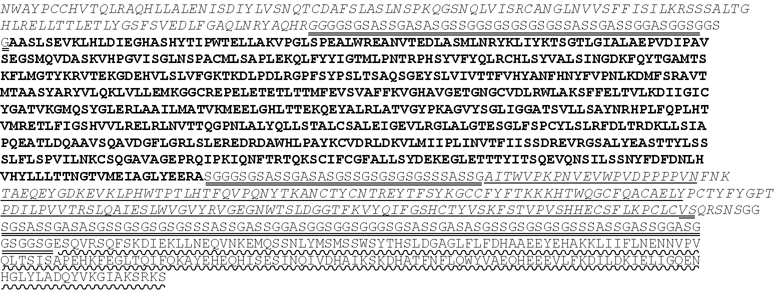 | 11 |
| Exemplary | MRAVGVFLAICLVTIFVLPTWG | 12 |
| mammalian | | |
| leader | | |
| sequence | | |
| Exemplary | MDSKGSSQKGSRLLLLLVVSNLLLPQGVLA | 13 |
| mammalian | | |
| leader | | |
| sequence | | |
| 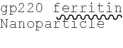 | 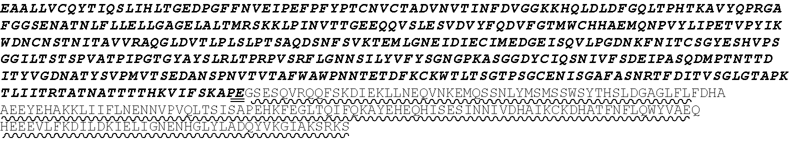 | 14 |
| Gp42 | DSKGSSQKGSRLLLLLLVVSNLLLPQGVLAYFLPPRVRGGGRVAAAAITWVPKNVEVWPVDPPPPVNFNKTAEQEYGDKE | 15 |
| | VKLPHWTPTLHTFQVPQNYTKANCTYCNTREYTFSYKGCCFYFTKKKHTWNGCFQACAELYPCTYFYGPTPDILPVVTRN | |
| | LNAIESLWVGVYRVGEGNWTSLDGGTFKVYQIFGSHCTYVSKFSTVPVSHHECSFLKPCLCVSQRSNS | |
| Exemplary gL | NWAYPCCHVTQLRAQHLLALENISDIYLVSNQTCDGFSLASLNSPKNGSNQLVISRCANGLNVVSFFISILKRSSSALTG | 16 |
| polypeptide | HLRELLTTLETLYGSFSVEDLFGANLNRYAWHRGG | |
| Exemplary gH | AASLSEVKLHLDIEGHASHYTIPWTELMAKVPGLSPEALWREANVTEDLASMLNRYKLIYKTSGTLGIALAEPVDIPAVS | 17 |
| polypeptide | EGSMQVDASKVHPGVISGLNSPACMLSAPLEKQLFYYIGTMLPNTRPHSYVFYQLRCHLSYVALSINGDKFQYTGAMTSK | |
| | FLMGTYKRVTEKGDEHVLSLVFGKTKDLPDLRGPFSYPSLTSAQSGDYSLVIVTTFVHYANFHNYFVPNLKDMFSRAVTM | |
| | TAASYARYVLQKLVLLEMKGGCREPELDTETLTTMFEVSVAFFKVGHAVGETGNGCVDLRWLAKSFFELTVLKDIIGICY | |
| | GATVKGMQSYGLERLAAMLMATVKMEELGHLTTEKQEYALRLATVGYPKAGVYSGLIGGATSVLLSAYNRHPLFQPLHTV | |
| | MRETLFIGSHVVLRELRLNVTTQGPNLALYQLLSTALCSALEIGEVLRGLALGTESGLFSPCYLSLRFDLTRDKLLSMAP | |
| | QEATLDQAAVSNAVDGFLGRLSLEREDRDAWHLPAYKCVDRLDKVLMIIPLINVTFIISSDREVRGSALYEASTTYLSSS | |
| | LFLSPVIMNKCSQGAVAGEPRQIPKIQNFTRTQKSCIFCGFALLSYDEKEGLETTTYITSQEVQNSILSSNYFDFDNLHV | |
| | HYLLLTTNGTVMEIAGLYEERA | |
| Exemplary | EAALLVCQYTIQSLIHLTGEDPGFFNVEIPEFPFYPTCNVCTADVNVTINFDVGGKKHQLKLKFGQLTPHTKAVYQPRGA | 18 |
| gp220 | FGGSENATNLFLLELLGAGELALTMRSKKLPINVTTGEEQQVSLESVDVYFQDVFGTMWCHHAEMQNPVYLIPETVPYIK | |
| polypeptide | WDNCNSTNITAVVRAQGLDVTLPLSLPTSAQDSNFSVKTEMLGNEIDIECIMEDGEISQVLPGDNKFNITCSGYESHVPS | |
| | GGILTSTSPVATPIPGTGYAYSLRLTPRPVSRFLGNNSILYVFYSGNGPKASGGDYCIQSNIVFSDEIPASQDMPTNTTD | |
| | ITYVGDNATYSVPMVTSEDANSPNVTVTAFWAWPNNTETDFKCKWTLTSGTPSGCENISGAFASNRTFDITVSGLGTAPK | |
| | TLIITRTATNATTTTHKVIFSKAPE | |
| Exemplary | gccaccatgagagctgtgggagtgttcctggccatctgcctggtcaccatcttcgtgctgcctacctggggcaactgggc | 19 |
| polynucleotide | ttacccttgctgtcatgtgacccagctgagagcccagcatctgctggccctggaaaacatctccgacatctacctggtgt | |
| encoding a | ccaaccagacctgcgacgccttctctctggcctctctgaactctcctaagcagggctccaaccagctcgtgatctccaga | |
| gL/gH/gp42/ | tgtgccaacggcctgaacgtggtgtccttcttcatctctatcctgaagcggtccagcagcgccctgaccggacatctgag | |
| ferritin | agagctgctgaccacactggaaaccctgtacggctccttcagcgtggaagatctgtttggcgcccagctgaacagatacg | |
| nanoparticle | cctggcatagaggtggcggcggatctggctctgcttcttctggtgcttctgcctctggatcctctggcggctctggttcc | |
| (C16 of SEQ ID | ggttctggaagcggatcttctgccagctctggcgcatcttctggcggagctagcggaggctctggtggtagcggagctgc | |
| NO: 9) | ttctctgtccgaagtgaagctgcacctggacatcgagggccacgccagccactatacaatcccttggactgagctgctgg | |
| | ccaaggtgccaggactgtctcctgaagctctttggcgcgaggccaacgtgaccgaggatctggcatctatgctgaaccgg | |
| | tacaagctgatctacaagaccagcggcaccctgggaatcgctctggctgagcctgtggatatccctgctgtgtccgaggg | |
| | ctctatgcaggtcgacgcttctaaagtgcaccccggcgtgatcagcggcctgaattctcctgcctgtatgctgagcgccc | |
| | ctctggaaaagcagctgttctactacatcggcaccatgctgcccaacaccagacctcactcctacgtgttctaccagctg | |
| | cggtgccacctgtcttacgtggccctgtctatcaacggcgacaagttccagtacaccggcgctatgacctccaagttcct | |
| | gatgggcacctacaagagagtgaccgagaagggcgacgagcatgtgctgtctctggtgttcggaaagaccaaggacctgc | |
| | ctgacctgagaggccccttcagctatccctctctgacctccgctcagtccggcgagtattccctggtcatcgtgaccacc | |
| | ttcgtgcactacgccaacttccacaactacttcgtgcccaacctgaaggacatgttctctcgggccgtgacaatgaccgc | |
| | cgcctcttacgctagatacgtgctgcagaaactggtgctgctggaaatgaagggcggctgcagagagcccgagctggaaa | |
| | cagagacactgaccaccatgttcgaggtgtccgtggccttcttcaaagtgggccatgctgtgggcgagacaggcaacgga | |
| | tgtgtggatctgagatggctggctaagagcttcttcgagctgaccgtgctgaaggatatcatcggcatctgctacggcgc | |
| | caccgtgaagggcatgcagtcttacggactggaaagactggccgccatcctgatggctaccgtgaagatggaagaactgg | |
| | gccacctgaccaccgagaagcaagagtacgctctgagactggccaccgtgggctatcctaaagccggcgtttactccgga | |
| | ctgatcggcggagcaacatccgtgctgctgtccgcctacaacagacaccctctgttccagcctctgcacaccgtgatgcg | |
| | cgagacactgttcatcggctctcacgtggtgctgcgcgagctgagactgaacgtgacaacccagggacctaatctggccc | |
| | tgtatcagctgctgagcaccgctctgtgtagcgctctggaaatcggcgaggtgctgagaggactggctctgggaacagag | |
| | tccggcctgttctccccttgctacctgtctctgagattcgacctgaccagagataagctgctgtctatcgcccctcaaga | |
| | ggctaccctggatcaggctgcagtgtctcaggccgtggatggctttctgggcagactgtccctggaaagagaggacaggg | |
| | acgcttggcatctgcccgcctataagtgcgtggacagactggacaaggtgctgatgatcattcccctgatcaatgtgacc | |
| | ttcatcatctcctccgaccgggaagtgcgaggctctgctctgtatgaggcctctactacctacctgtcctccagcctgtt | |
| | tctgtcccctgtgatcctgaacaagtgctcccaaggcgctgtggccggcgagcctagacagatccctaagatccagaact | |
| | tcacccggactcagaagtcctgtatcttctgcggcttcgccctgctgtcctacgacgagaaagaaggcctggaaaccacc | |
| | acctacatcaccagccaagaggtgcagaactccatcctgtccagcaattacttcgacttcgacaacctgcatgtgcacta | |
| | cctgctgctgactaccaacggcacagtgatggaaatcgccggcctgtacgaggaaagagcatccggcggaggttctggca | |
| | gtgctagtagtggcgcttccgcttctggctcctccggctctggatctggaagtggctcctctagtgcttccagcggcgct | |
| | atcacatgggtgcccaagcctaacgtggaagtgtggccagtggatcctcctccacctgtgaacttcaacaagaccgccga | |
| | gcaagaatacggggacaaagaagtgaaactgccccactggacccctacactgcacacctttcaggtgccccagaactaca | |
| | ccaaggccaactgcacctactgcaatacccgcgagtacacctttagctacaagggctgctgcttctacttcaccaagaag | |
| | aaacacacctggcagggctgctttcaggcctgtgctgagctgtacccctgtacctacttctacggccccacacctgacat | |
| | cctgccagtggtcacaagatccctgcaggccatcgagtctctgtgggtcggagtgtacagagtcggcgaaggcaactgga | |
| | cctctctggatggcggaaccttcaaggtgtaccagatcttcggctcccattgcacctacgtgtccaagttctccaccgtg | |
| | cctgtgtctcaccacgagtgcagctttctgaagccctgcctgtgcgtgtcccagagatctaatagcggcggcagtggctc | |
| | tgcatctagtggcgcaagtgcttccggatcttccggctcaggctccggtagtggatcttctagtgcctcttccggcgctt | |
| | ctagcggtggtgcatctggtggatctggcggaagcggcggaggaagtggatcagcaagttcaggcgcttctgctagcggc | |
| | tctagtggcagtggcagcggttccggatcctcttcagcatcatctggcgctagttctggtggcgccagtggcggctccgg | |
| | tggttctggcgaatctcaagttcggtcccagttcagcaaggatatcgaaaagctgctgaacgagcaagtgaacaaagaga | |
| | tgcagtccagcaacctgtatatgtccatgtccagctggtcctacacacactccctcgatggcgctggcctgttcctgttt | |
| | gatcacgctgccgaggaatacgagcacgccaagaagctgatcatcttcctgaacgagaacaacgtgcccgtgcagctgac | |
| | ctccatctctgcccctgagcacaagttcgagggcctgacacagatctttcagaaggcctacgaacacgagcagcacatca | |
| | gcgagtccatcaaccagatcgtggaccacgccatcaagtccaaggatcacgccaccttcaactttctgcagtggtacgtg | |
| | gccgagcagcatgaggaagaggtgctgttcaaggacatcctggacaagatcgagctgatcggccaagagaaccatggcct | |
| | gtacctggccgaccagtacgtgaagggaatcgccaagtctcggaagtcctga | |
| Exemplary | gccaccatgagagctgtgggagtgttcctggccatctgcctggtcaccatcttcgtgctgcctacctggggcaactgggc | 20 |
| polynucleotide | ttacccttgctgtcatgtgacccagctgagagcccagcatctgctggccctggaaaacatctccgacatctacctggtgt | |
| encoding a | ccaaccagacctgcgacgccttctctctggcctctctgaactctcctaagcagggctccaaccagctcgtgatctccaga | |
| gL/gH/gp42/ | tgtgccaacggcctgaacgtggtgtccttcttcatctctatcctgaagcggtccagcagcgccctgaccggacatctgag | |
| ferritin | agagctgctgaccacactggaaaccctgtacggctccttcagcgtggaagatctgtttggcgcccagctgaacagatacg | |
| nanoparticle | cctggcatagaggtggcggcggatctggctctgcttcttctggtgcttctgcctctggatcctctggcggctctggttcc | |
| (C17 of SEQ ID | ggttctggaagcggatcttctgccagctctggcgcatcttctggcggagctagcggaggctctggtggtagcggagctgc | |
| NO: 10) | ttctctgtccgaagtgaagctgcacctggacatcgagggccacgccagccactatacaatcccttggactgagctgctgg | |
| | ccaaggtgccaggactgtctcctgaagctctttggcgcgaggccaacgtgaccgaggatctggcatctatgctgaaccgg | |
| | tacaagctgatctacaagaccagcggcaccctgggaatcgctctggctgagcctgtggatatccctgctgtgtccgaggg | |
| | ctctatgcaggtcgacgcttctaaagtgcaccccggcgtgatcagcggcctgaattctcctgcctgtatgctgagcgccc | |
| | ctctggaaaagcagctgttctactacatcggcaccatgctgcccaacaccagacctcactcctacgtgttctaccagctg | |
| | cggtgccacctgtcttacgtggccctgtctatcaacggcgacaagttccagtacaccggcgctatgacctccaagttcct | |
| | gatgggcacctacaagagagtgaccgagaagggcgacgagcatgtgctgtctctggtgttcggaaagaccaaggacctgc | |
| | ctgacctgagaggccccttcagctatccctctctgacctccgctcagtccggcgagtattccctggtcatcgtgaccacc | |
| | ttcgtgcactacgccaacttccacaactacttcgtgcccaacctgaaggacatgttctctcgggccgtgacaatgaccgc | |
| | cgcctcttacgctagatacgtgctgcagaaactggtgctgctggaaatgaagggcggctgcagagagcccgagctggaaa | |
| | cagagacactgaccaccatgttcgaggtgtccgtggccttcttcaaagtgggccatgctgtgggcgagacaggcaacgga | |
| | tgtgtggatctgagatggctggctaagagcttcttcgagctgaccgtgctgaaggatatcatcggcatctgctacggcgc | |
| | caccgtgaagggcatgcagtcttacggactggaaagactggccgccatcctgatggctaccgtgaagatggaagaactgg | |
| | gccacctgaccaccgagaagcaagagtacgctctgagactggccaccgtgggctatcctaaagccggcgtttactccgga | |
| | ctgatcggcggagcaacatccgtgctgctgtccgcctacaacagacaccctctgttccagcctctgcacaccgtgatgcg | |
| | cgagacactgttcatcggctctcacgtggtgctgcgcgagctgagactgaacgtgacaacccagggacctaatctggccc | |
| | tgtatcagctgctgagcaccgctctgtgtagcgctctggaaatcggcgaggtgctgagaggactggctctgggaacagag | |
| | tccggcctgttctccccttgctacctgtctctgagattcgacctgaccagagataagctgctgtctatcgcccctcaaga | |
| | ggctaccctggatcaggctgcagtgtctcaggccgtggatggctttctgggcagactgtccctggaaagagaggacaggg | |
| | acgcttggcatctgcccgcctataagtgcgtggacagactggacaaggtgctgatgatcattcccctgatcaatgtgacc | |
| | ttcatcatctcctccgaccgggaagtgcgaggctctgctctgtatgaggcctctactacctacctgtcctccagcctgtt | |
| | tctgtcccctgtgatcatgaacaagtgctcccagggcgcagtggccggcgaacctagacagatccctaagatccagaact | |
| | tcacccggactcagaagtcctgtatcttctgcggcttcgccctgctgtcctacgacgagaaagaaggcctggaaaccacc | |
| | acctacatcaccagccaagaggtgcagaactccatcctgtccagcaattacttcgacttcgacaacctgcatgtgcacta | |
| | cctgctgctgactaccaacggcacagtgatggaaatcgccggcctgtacgaggaaagagcatccggcggaggttctggca | |
| | gtgctagtagtggcgcttccgcttctggctcctccggctctggatctggaagtggctcctctagtgcttccagcggcgct | |
| | atcacatgggtgcccaagcctaacgtggaagtgtggccagtggatcctcctccacctgtgaacttcaacaagaccgccga | |
| | gcaagaatacggggacaaagaagtgaaactgccccactggacccctacactgcacacctttcaggtgccccagaactaca | |
| | ccaaggccaactgcacctactgcaatacccgcgagtacacctttagctacaagggctgctgcttctacttcaccaagaag | |
| | aaacacacctggcagggctgctttcaggcctgtgctgagctgtacccctgtacctacttctacggccccacacctgacat | |
| | cctgccagtggtcaccagaaacctgaacgccatcgagtccctgtgggtcggagtgtacagagtcggcgaaggcaactgga | |
| | cctctctggatggcggaaccttcaaggtgtaccagatcttcggctcccattgcacctacgtgtccaagttctccaccgtg | |
| | cctgtgtctcaccacgagtgcagctttctgaagccctgcctgtgcgtgtcccagagatctaatagcggcggcagtggctc | |
| | tgcatctagtggcgcaagtgcttccggatcttccggctcaggctccggtagtggatcttctagtgcctcttccggcgctt | |
| | ctagcggtggtgcatctggtggatctggcggaagcggcggaggaagtggatcagcaagttcaggcgcttctgctagcggc | |
| | tctagtggcagtggcagcggttcaggatcttcctcagcaagcagcggagcatcaagtggtggtgccagcggaggatcagg | |
| | cggttctggcgaatctcaagtgcggtcccagttcagcaaggatatcgaaaagctgctgaacgagcaagtgaacaaagaga | |
| | tgcagtccagcaacctgtatatgtccatgtccagctggtcctacacacactcccttgatggcgctggcctgttcctgttt | |
| | gaccacgccgctgaggaatacgagcacgccaagaagctgatcatcttcctgaacgagaacaacgtgcccgtgcagctgac | |
| | ctccatctctgcccctgagcacaagttcgagggcctgacacagatctttcagaaggcctacgaacacgagcagcacatca | |
| | gcgagtccatcaaccagatcgtggaccacgctatcaagtccaaggaccacgccaccttcaactttctgcagtggtacgtg | |
| | gccgagcagcatgaggaagaggtgctgttcaaggacatcctggacaagatcgagctgatcggccaagagaaccatggcct | |
| | gtacctggccgaccagtacgtgaagggaatcgccaagtctcggaagtcctga | |
| Exemplary | gccaccatgagagctgtgggagtgttcctggccatctgcctggtcaccatcttcgtgctgcctacctggggcaactgggc | 21 |
| polynucleotide | ttacccttgctgtcatgtgacccagctgagagcccagcatctgctggccctggaaaacatctccgacatctacctggtgt | |
| encoding a | ccaaccagacctgcgacgccttctctctggcctctctgaactctcctaagcagggctccaaccagctcgtgatctccaga | |
| gL/gH/gp42/ | tgtgccaacggcctgaacgtggtgtccttcttcatctctatcctgaagcggtccagcagcgccctgaccggacatctgag | |
| ferritin | agagctgctgaccacactggaaaccctgtacggctccttcagcgtggaagatctgtttggcgcccagctgaacagatacg | |
| nanoparticle | cctggcatagaggtggcggcggatctggctctgcttcttctggtgcttctgcctctggatcctctggcggctctggttcc | |
| (C18 of SEQ ID | ggttctggaagcggatcttctgccagctctggcgcatcttctggcggagctagcggaggctctggtggtagcggagctgc | |
| NO: 11) | ttctctgtccgaagtgaagctgcacctggacatcgagggccacgccagccactatacaatcccttggactgagctgctgg | |
| | ccaaggtgccaggactgtctcctgaagctctttggcgcgaggccaacgtgaccgaggatctggcatctatgctgaaccgg | |
| | tacaagctgatctacaagaccagcggcaccctgggaatcgctctggctgagcctgtggatatccctgctgtgtccgaggg | |
| | ctctatgcaggtcgacgcttctaaagtgcaccccggcgtgatcagcggcctgaattctcctgcctgtatgctgagcgccc | |
| | ctctggaaaagcagctgttctactacatcggcaccatgctgcccaacaccagacctcactcctacgtgttctaccagctg | |
| | cggtgccacctgtcttacgtggccctgtctatcaacggcgacaagttccagtacaccggcgctatgacctccaagttcct | |
| | gatgggcacctacaagagagtgaccgagaagggcgacgagcatgtgctgtctctggtgttcggaaagaccaaggacctgc | |
| | ctgacctgagaggccccttcagctatccctctctgacctccgctcagtccggcgagtattccctggtcatcgtgaccacc | |
| | ttcgtgcactacgccaacttccacaactacttcgtgcccaacctgaaggacatgttctctcgggccgtgacaatgaccgc | |
| | cgcctcttacgctagatacgtgctgcagaaactggtgctgctggaaatgaagggcggctgcagagagcccgagctggaaa | |
| | cagagacactgaccaccatgttcgaggtgtccgtggccttcttcaaagtgggccatgctgtgggcgagacaggcaacgga | |
| | tgtgtggatctgagatggctggctaagagcttcttcgagctgaccgtgctgaaggatatcatcggcatctgctacggcgc | |
| | caccgtgaagggcatgcagtcttacggactggaaagactggccgccatgctgatggctaccgtgaagatggaagaactgg | |
| | gccacctgaccaccgagaagcaagagtacgctctgagactggccaccgtgggctatcctaaagccggcgtttactccgga | |
| | ctgatcggcggagcaacatccgtgctgctgtccgcctacaacagacaccctctgttccagcctctgcacaccgtgatgcg | |
| | cgagacactgttcatcggctctcacgtggtgctgcgcgagctgagactgaacgtgacaacccagggacctaatctggccc | |
| | tgtatcagctgctgagcaccgctctgtgtagcgctctggaaatcggcgaggtgctgagaggactggctctgggaacagag | |
| | tccggcctgttctccccttgctacctgtctctgagattcgacctgaccagagataagctgctgtctatggcccctcaaga | |
| | ggctaccctggatcaggccgcagtgtctaatgccgtggatggctttctgggcagactgtccctggaaagagaggacaggg | |
| | acgcttggcatctgcccgcctataagtgcgtggacagactggacaaggtgctgatgatcattcccctgatcaatgtgacc | |
| | ttcatcatctcctccgaccgggaagtgcgaggctctgctctgtatgaggcctctactacctacctgtcctccagcctgtt | |
| | tctgtcccctgtgatcatgaacaagtgctcccagggcgcagtggccggcgaacctagacagatccctaagatccagaact | |
| | tcacccggactcagaagtcctgtatcttctgcggcttcgccctgctgtcctacgacgagaaagaaggcctggaaaccacc | |
| | acctacatcaccagccaagaggtgcagaactccatcctgtccagcaattacttcgacttcgacaacctgcatgtgcacta | |
| | cctgctgctgactaccaacggcacagtgatggaaatcgccggcctgtacgaggaaagagcatccggcggaggttctggca | |
| | gtgctagtagtggcgcttccgcttctggctcctccggctctggatctggaagtggctcctctagtgcttccagcggcgct | |
| | atcacatgggtgcccaagcctaacgtggaagtgtggccagtggatcctcctccacctgtgaacttcaacaagaccgccga | |
| | gcaagaatacggggacaaagaagtgaaactgccccactggacccctacactgcacacctttcaggtgccccagaactaca | |
| | ccaaggccaactgcacctactgcaatacccgcgagtacacctttagctacaagggctgctgcttctacttcaccaagaag | |
| | aaacacacctggaacggctgctttcaggcctgcgctgagctgtacccctgtacctacttctacggccccacacctgacat | |
| | cctgccagtggtcaccagaaacctgaacgccatcgagtccctgtgggtcggagtgtacagagtcggcgaaggcaactgga | |
| | cctctctggatggcggcaccttcaaggtgtaccagatcttcggctcccattgcacctacgtgtccaagttctccaccgtg | |
| | cctgtgtctcaccacgagtgcagctttctgaagccctgcctgtgcgtgtcccagagatctaatagcggcggcagtggctc | |
| | tgcatctagtggcgcaagtgcttccggatcttccggctcaggcagtggttctggctctagctcagcttcttccggcgcaa | |
| | gttcaggcggtgcatctggtggatctggcggaagcggcggaggaagtggatctgcttctagcggagcatccgcctccgga | |
| | tcatcaggatctggatccggtagcggctccagctctgcaagtagtggtgcctcaagcggaggtgccagcggtggcagcgg | |
| | aggatccggcgaatctcaagttcggtcccagttcagcaaggatatcgaaaagctgctgaacgagcaagtgaacaaagaga | |
| | tgcagtccagcaacctgtatatgtccatgtccagctggtcctacacacactcccttgatggcgctggcctgttcctgttt | |
| | gaccacgccgctgaggaatacgagcacgccaagaaactgatcatcttcctgaacgagaacaacgtgcccgtgcagctgac | |
| | ctccatctctgcccctgagcacaagttcgagggcctgacacagatctttcagaaggcctacgaacacgagcagcacatca | |
| | gcgagtccatcaaccagatcgtggaccacgctatcaagtccaaggaccacgccaccttcaactttctgcagtggtacgtg | |
| | gccgagcagcatgaggaagaggtgctgttcaaggacatcctggacaagatcgagctgatcggccaagagaaccatggcct | |
| | gtacctggccgaccagtacgtgaagggaatcgccaagtctcggaagtcctga | |
| Exemplary | atggattctaagggctccagccagaagggctctagactgctgctgctcctggtggtgtctaacctcctgttgcctcaggg | 22 |
| polynucleotide | cgtgctggctgaagctgctctgctcgtgtgccagtacaccatccagagcctgatccatctgaccggcgaggaccctggct | |
| encoding a | tcttcaacgtggaaatccctgagttccctttctaccctacctgcaacgtgtgcaccgccgacgtgaacgtgaccatcaac | |
| gp220/ferritin | tttgatgtcggcggcaagaagcaccagctggacctggattttggccagctgacccctcacaccaaggccgtgtatcaacc | |
| nanoparticle | tagaggcgcctttggcggctccgagaacgccaccaatctgtttctgctggaactgctcggagctggcgagctggctctga | |
| (SEQ ID NO: | ccatgcggtctaagaaactgcccatcaatgtgaccacaggcgaggaacagcaggtttccctggaatccgtggacgtgtac | |
| 14) | ttccaagacgtgttcggcaccatgtggtgccaccacgccgagatgcagaaccccgtgtatctgatccccgagacagtgcc | |
| | ctacatcaagtgggacaactgcaactccaccaacatcaccgccgtcgtcagagcccaaggcctggatgttacactgcccc | |
| | tgagcctgcctacctccgctcaggactctaacttctccgtgaaaaccgagatgctgggcaacgagatcgacatcgagtgc | |
| | atcatggaagatggcgagatctcccaggtgctgcccggcgacaacaagttcaatatcacctgttccggctacgagtctca | |
| | cgtgccatctggcggcatcctgacctctacaagccctgtggctacccctattcctggcaccggctacgcttactccctga | |
| | gactgacacctcggcctgtgtccagattcctgggcaacaactccatcctgtacgtgttctactccggcaacggccctaag | |
| | gcttctggcggcgattactgcatccagtccaacatcgtgttctccgacgagatccccgccagccaggatatgcctaccaa | |
| | caccaccgatatcacctacgtgggcgacaacgccacctactccgtgcctatggtcacctccgaggatgccaactctccca | |
| | acgtgaccgtgacagccttttgggcctggcctaacaacaccgagacagacttcaagtgcaagtggaccctgaccagcggc | |
| | accccatctggctgcgagaatatctctggcgccttcgcctctaaccggaccttcgacatcaccgtgtctggcctgggaac | |
| | cgctcctaagacactgatcatcacccggaccgccacaaatgctaccaccaccactcacaaagtgatcttcagcaaggccc | |
| | ctgagggcagcgaatctcaagtgcggcagcagttctccaaggacatcgagaagctgctgaacgagcaagtgaacaaagag | |
| | atgcagtcctccaacctgtatatgtccatgtcctcttggagctacacccacagcctggatggcgctggcctgttcctgtt | |
| | tgatcacgccgctgaggaatacgagcacgccaagaagctgatcatcttcctgaacgagaacaacgtgccagtgcagctga | |
| | cctccatctctgccccagagcacaagttcgagggcctgacacagatcttccagaaggcctacgaacacgaacagcacatc | |
| | tccgagtccatcaacaatatcgtggaccacgccattaagtgcaaggatcacgccaccttcaactttctgcagtggtacgt | |
| | ggccgagcagcacgaggaagaggtgctgtttaaggacatcctggacaagatcgagctgatcggcaatgagaaccacggcc | |
| | tgtacctggccgaccagtacgtgaagggaatcgccaagagccggaagtcctga | |
| Exemplary H. | FSKDIEKLLNEQVNKEMQSSNLYMSMSSWSYTHSLDGAGLFLFDHAAEEYEHAKKLIIFLNENNVPVQLTSISAPEHKFE | 23 |
| pylori | GLTQIFQKAYEHEQHISESINQIVDHAIKSKDHATFNFLQWYVAEQHEEEVLFKDILDKIELIGQENHGLYLADQYVKGI | |
| ferritin | AKSRKS | |
| Exemplary | ESQVRSQFSKDIEKLLNEQVNKEMQSSNLYMSMSSWSYTHSLDGAGLFLFDHAAEEYEHAKKLIIFLNENNVPVQLTSIS | 24 |
| Bullfrog H. | APEHKFEGLTQIFQKAYEHEQHISESINQIVDHAIKSKDHATFNFLQWYVAEQHEEEVLFKDILDKIELIGQENHGLYLA | |
| pylori | DQYVKGIAKSRKS | |
| ferritin | | |
| Gp220 with | EAALLVCQYTIQSLIHLTGEDPGFFNVEIPEFPFYPTCNVCTADVNVTINFDVGGKKHQLDLDFGQLTPHTKAVYQPRGA | 25 |
| ferritin of | FGGSENATNLFLLELLGAGELALTMRSKKLPINVTTGEEQQVSLESVDVYFQDVFGTMWCHHAEMQNPVYLIPETVPYIK | |
| SEQ ID NO: 24 | WDNCNSTNITAVVRAQGLDVTLPLSLPTSAQDSNFSVKTEMLGNEIDIECIMEDGEISQVLPGDNKFNITCSGYESHVPS | |
| | GGILTSTSPVATPIPGTGYAYSLRLTPRPVSRFLGNNSILYVFYSGNGPKASGGDYCIQSNIVFSDEIPASQDMPTNTTD | |
| | ITYVGDNATYSVPMVTSEDANSPNVYVTAFWAWPNNTETDFKCKWTLTSGTPSGCENISGAFASNRTFDITVSGLGTAPK | |
| | TLIITRTANNATTTTHKVIFSKAPEGSESQVRSQFSKDIEKLLNEQVNKEMQSSNLYMSMSSWSYTHSLDGAGLFLFDHA | |
| | AEEYEHAKKLIIFLNENNVPVQLTSISAPEHKFEGLTQIFQKAYEHEQHISESINQIVDHAIKSKDHATFNFLQWYVAEQ | |
| | HEEEVLFKDILDKIELIGQENHGLYLADQYVKGIAKSRKS | |
| 4 amino acid | GGGS | 26 |
| linker | | |
| 8 amino acid | GGGSGGGS | 27 |
| linker | | |
| 20 amino acid | GGGSGGGSGGGSGGGSGGGS | 28 |
| linker | | |
| 47 amino acid | SGGGSGSASSGASASGSSCSGSGSGSSSASSGASSGGASGGGSGGSG | 29 |
| linker | | |
| 46 amino acid | GGSGSASSGASASGSSNGSGSGSGSNSSASSGASSGGASGGSGGSG | 30 |
| linker | | |
| 16 amino acid | GGGGSGGGGSGGGGSG | 31 |
| linker | | |
| 28 amino acid | GGSGSGSNSSASSGASSGGASGGSGGSG | 32 |
| linker | | |
| 26 amino acid | GGSGSASAEAAAKEAAAKAGGSGGSG | 33 |
| linker (FR1 | | |
| linker) | | |
| 32 amino acid | GGSGSASAEAAAKEAAAKEAAAKASGGSGGSG | 34 |
| linker (FR2 | | |
| linker) | | |
| 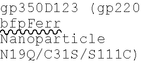 | 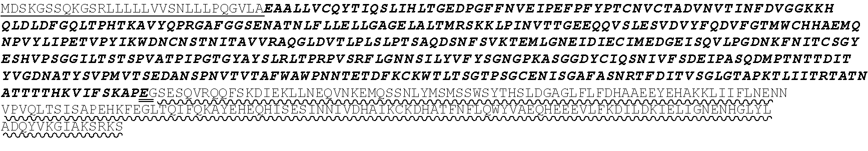 | 35 |
| C16 gL/gH/gp42/ ferritin including the leader of SEQ ID NO: 12 | 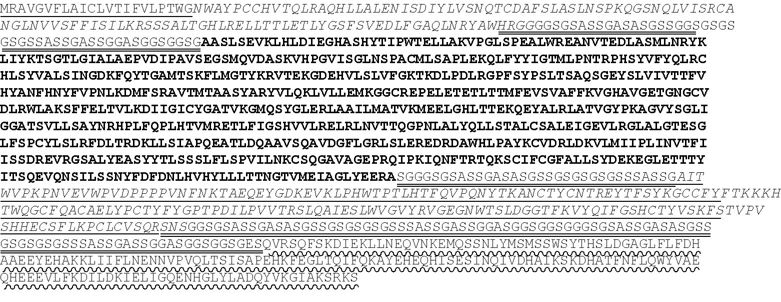 | 36 |
| C16 gL/gH/gp42/ ferritin including the leader of SEQ ID NO: 13 | 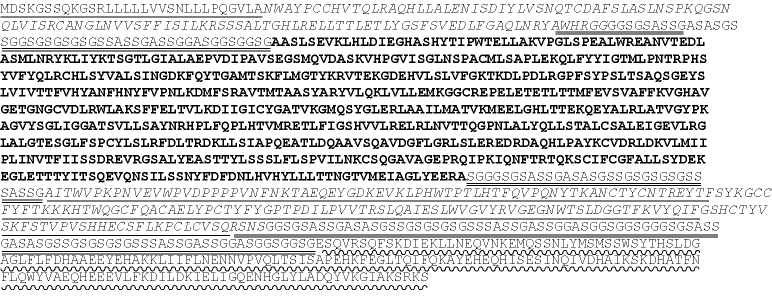 | 37 |
| C17 gL/gH/gp42/ ferritin including the leader of SEQ ID NO: 12 | 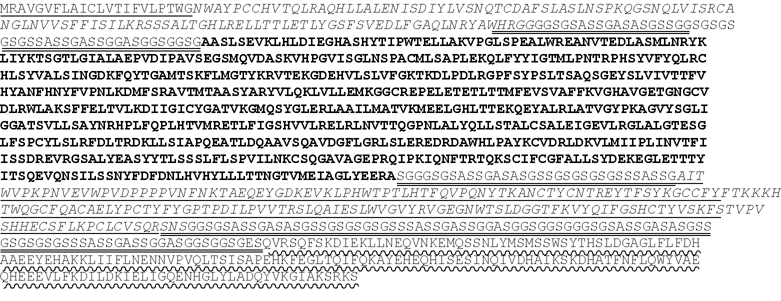 | 38 |
| C17 gL/gH/gp42/ ferritin including the leader of SEQ ID NO: 13 | 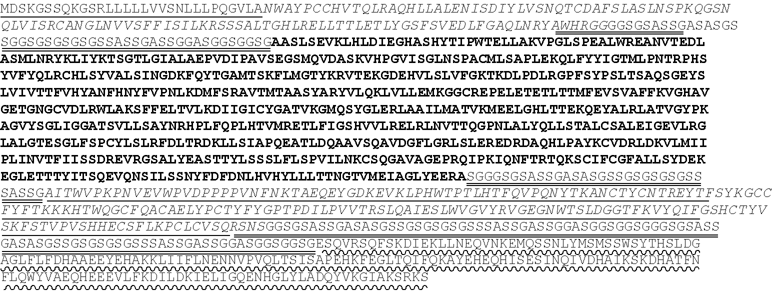 | 39 |
| C18 gL/gH/gp42/ ferritin including the leader of SEQ ID NO: 12 | 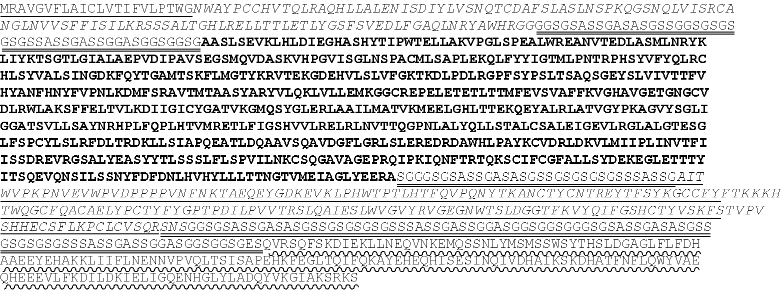 | 40 |
| C18 gL/gH/gp42/ ferritin including the leader of SEQ ID NO: 13 | 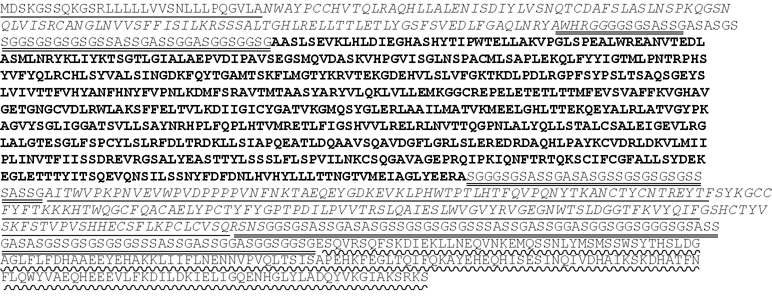 | 41 |
| Exemplary | FSKDIEKLLNEQVNKEMQSSNLYMSMSSWCYTHSLDGAGLFLFDHAAEEYEHAKKLIIFLNENNVPVQLTSISAPEHKFE | 42 |
| H. pylori | GLTQIFQKAYEHEQHISESINQIVDHAIKSKDHATFNFLQWYVAEQHEEEVLFKDILDKIELIGQENHGLYLADQYVKGI | |
| ferritin | AKSRKS | |
| Exemplary | FSKDIEKLLNEQVNKEMNSSNLYMSMSSWSYTHSLDGAGLFLFDHAAEEYEHAKKLIIFLNENNVPVQLTSISAPEHKFE | 43 |
| H. pylori | GLTQIFQKAYEHEQHISESINQIVDHAIKSKDHATFNFLQWYVAEQHEEEVLFKDILDKIELIGQENHGLYLADQYVKGI | |
| ferritin | AKSRKS | |

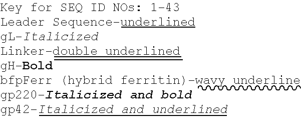

### EXAMPLES

The following examples are provided to illustrate certain disclosed embodiments and are not to be construed as limiting the scope of this disclosure in any way.

### Example 1: Generation of Host Cells Producing C16, C17, and C18 Constructs (Nanoparticles)

Fusion proteins (antigenic polypeptides) that elicit antibodies against EBV were developed. Self-assembling ferritin nanoparticles were developed that display EBV gH, gL, and gp42 polypeptides as a single chain.

CHO cells producing the EBV Ferritin gp42_gH_gL pre-candidates C16, C17, and C18 (SEQ ID NOs: 9-11, respectively, as provided in Table 2) were generated by expressing the respective recombinant fusion protein in the in-house derived CHO DXB11 8D6 host cell line. CHO cell pools were generated using Cell Line Development's CD (chemically defined), ADC (animal derived component)-free cell line development platform. CHO cell pools were generated and cryopreserved as research cell banks (RCBs). The pools were also assayed for protein titer.

### Materials and Methods

The following plasmid constructs were used in this Example: plasmid DNA encoding the codon optimized EBV Ferritin gp42_gH_gL_C16 (Thermo Fisher Scientific construct, lot #2647668); plasmid DNA encoding the codon optimized EBV Ferritin gp42_gH_gL_C17 (Thermo Fisher Scientific construct, lot #2647665); and plasmid DNA encoding the codon optimized EBV Ferritin gp42_gH_gL_C18: (Thermo Fisher Scientific construct, lot #2647662).

CHO DXB11 8D6 parental (host) cells were thawed, cultured, and transfected (via electroporation) with one of the above-mentioned plasmids, and the produced particles (C16, C17, and C18) were harvested. Prior to harvest, transfected cells were selected using methotrexate (MTX). Cells were passaged in CD CHO supplemented with 4 mM L-Gln and MTX (2 rounds of selection).

Cell culture raw materials used included CD CHO medium, L-Glutamine, CD DG44 medium, Pluronic F-68, DMSO, and Methotrexate. Cell culture media harvests were clarified by centrifugation. The clarified harvests were analyzed for EBV Ferritin gp42 titer using the Octet Red 96 (US Breakthrough Lab, Cambridge).

### Results and Discussion

EBV Ferritin gp42 Cell Line Development: Transfections and Generation of CHO Cell Pools

The EBV Ferritin gp42 gH_gL_C16, gp42 gH_gL_C17, and gp42 gH_gL_C18 amino acid sequences were sent to Thermo Fisher Scientific for DNA sequence optimization and synthesis. Codon optimization changed only the DNA sequence for the ORF, leaving the amino acid sequence unchanged. As part of the optimization of the ORF, a consensus Kozak sequence was included at the 5′ end of the coding sequence. Restriction endonuclease (RE) sites were added to the extreme 5′ and 3′ ends of the coding sequences to facilitate molecular cloning.

The optimized transgene expression cassette containing the ORFs and the customized flanking regions were subcloned into the vector backbone pGZ729 (see, for example, U.S. Pat. No. 10,317,329 B2 for the pGZ729 backbone). Transfection quality DNA was prepared for these plasmids.

Each plasmid DNA was linearized by restriction endonuclease digestion and prepared for transfection under sterile conditions. The linearized plasmid DNA was transfected into the CHO DXB11 8D6 parental cell line by electroporation. Five replicate pools of transfected cells were generated. Immediately following transfection, cells were seeded in the parental cell growth medium (CD DG44/4 mM L-glutamine/0.18% (v/v) Pluronic® F-68) for recovery.

Two days post-transfection, transfected cell pools began DHFR-based selection process (two rounds of MTX selection). Throughout the selection process, the cell count was very low and, therefore, pools had to be combined during passage. For example, Pool C16 1/2/3 is a pool formed by the combining of pools 1, 2, and 3. At the completion of each round of selection, all pools were cryopreserved. For determination of EBV Ferritin gp42 production levels, pools were seeded into batch cultures in spin tubes. The batch culture parameters are summarized in Table 3. Media harvests from day 5 were assayed for titer. The day 5 titer for each pool is shown in Table 4.

TABLE 3
Spin tube cell culture conditions for titer determination

| Seed Density | 1 × 10⁶ | vc/mL |
|---|---|---|
| Seed Volume | 10 | mL |
| Seed Medium | 70% CD CHO/30% Efficient Feed B, | |
| | supplemented with 4 mM L-glutamine | |
| Temperature | 37° | C. |
| CO₂ | 5% | |
| Relative Humidity | 80% | |
| Shaking Speed | 260 | RPM |
| Orbit diameter | 50 | mm |

TABLE 4
EBV Ferritin gp42 pool titers in 5-day batch culture.

Day 5 Titer (g/L)

| Pool replicate | 5 nM MTX | 50 nM MTX |
|---|---|---|
| C16 1/2/3 | 0.048 | 0.02 |
| C16 4/5 | 0.048 | 0.02 |
| C17 1/2/4 | 0.063 | 0.015 |
| C17 3/5 | 0.071 | 0.022 |
| C18 1/2/3 | 0.069 | 0.028 |
| C18 4/5 | 0.082 | 0.05 |

CHO pools expressing EBV Ferritin gp42 were generated using an ADC-free CHO cell line development platform and cryopreserved as RCBs. For each candidate molecule, the 5 nM MTX pools had higher titers than the 50 nM MTX pools (as determined by Octet analysis).

### Example 2: Generation of Host Cells Producing gp220-Ferritin Nanoparticles

CHO cells producing EBV Ferritin gp220 bfp_Ferr (LC81213) were generated by expressing the recombinant fusion protein in the in-house derived CHO DXB11 8D6 host cell line. The amino acid sequence for the gp220 bfp_Ferr protein was used to construct the corresponding expression vector for transfection. Cell lines were generated using Cell Line Development's CD (chemically defined), ADC (animal derived component)-free cell line development platform. CHO cell pools were generated and, subsequently, CHO clones were isolated and screened.

### Materials and Methods

Plasmid DNA encoding the codon optimized EBV Ferritin gp220_bfpFerr (Thermo Fisher Scientific construct, lot #2349061) was used in this Example. CHO DXB11 8D6 parental (host) cells were thawed, cultured, and transfected (via electroporation) with the above-mentioned plasmid, and the produced particle (SEQ ID NO: 14, as provided in Table 2) was harvested. Prior to harvest, transfected cells were selected using methotrexate (MTX). Cells were passaged in CD CHO supplemented with 4 mM L-Gln and MTX (2 rounds of selection). Specifically, cell culture raw materials used were: CD CHO medium, L-Glutamine, CD DG44 medium, Pluronic F-68, DMSO, Methotrexate, EfficientFeed B, DMEM:F12, and HEPES.

For flow cytometric sorting and analysis, FITC-labeled anti-CD52 was used. In FACS, the top CD52 reporter expressing cells were collected from pool(s) and 1 cell per well was deposited in 96-well plates. For clone screening, selection, and expansion, plated wells were imaged on day 0 and each day following cell deposition. Selected clones (using methotrexate as described above) were expanded using spin tube and shake flask cultures. Cell culture media harvests were clarified by centrifugation. The clarified harvests were analyzed for EBV Ferritin gp220 titer using the Octet Red 96 (US Breakthrough Lab, Cambridge).

### Results and Discussion

EBV Ferritin gp220 Cell Line Development: Transfections and Generation of CHO Cell Pools

The EBV Ferritin gp220 bfp_Ferr (LC81213) amino acid sequence was sent to Thermo Fisher Scientific for DNA sequence optimization and synthesis. Codon optimization changed only the DNA sequence for the ORF, leaving the amino acid sequence (SEQ ID NO: 35) unchanged. As part of the optimization of the ORF, a consensus Kozak sequence was included at the 5′ end of the coding sequence. Restriction endonuclease (RE) sites were added to the extreme 5′ and 3′ ends of the coding sequences to facilitate molecular cloning.

The optimized transgene expression cassette containing the ORF and the customized flanking regions was subcloned into the vector backbone pGZ729. Transfection quality DNA was prepared for this plasmid.

Each plasmid DNA was linearized by restriction endonuclease digestion and prepared for transfection under sterile conditions. The linearized plasmid DNA was transfected into the CHO DXB11 8D6 parental cell line by electroporation. Five replicate pools of transfected cells were generated. Immediately following transfection, cells were seeded in the parental cell growth medium (CD DG44/4 mM L-glutamine/0.18% (v/v) Pluronic® F-68) for recovery.

Two days post-transfection, transfected cell pools began DHFR-based selection process (two rounds of MTX selection). At the completion of each round of selection, all pools were cryopreserved. For determination of EBV Ferritin gp220 production levels, pools were seeded into batch cultures in spin tubes. The batch culture parameters are summarized in Table 3 above. Media harvests from Day 5 were assayed for titer. The Day 5 titer for each pool is shown in Table 5.

TABLE 5
EBV Ferritin gp220 pool titers in 5-day batch culture.

Day 5 Titer (g/L)

| Pool replicate | 5 nM MTX | 50 nM MTX |
|---|---|---|
| 1 | 0.08 | 0.085 |
| 2 | 0.065 | 0.08 |
| 3 | 0.138 | 0.139 |
| 4 | 0.017 | 0.012 |
| 5 | 0.018 | 0.156 |

### Generation of CHO Cell Clones

Pools 5, 3, and 2 (50 nM MTX stage) were chosen for clone isolation. One vial of each pool was thawed and expanded for clone generation. Cells were expanded in MTX-free medium (CD CHO supplemented with 4 mM L-glutamine) for 1 week prior to the sort day. For clone isolation, cells from each pool were incubated with an ADC-free FITC-labeled anti-CD52 antibody (CLD, Sanofi) and sorted using the BD Influx with the Automated Cell Deposition Unit (ACDU). Each sort targeted a top percentage of CD52 expressing cells for collection. Cells isolated by sorting from Pools 5, 3, and 2 were deposited (by the ACDU) at 1 cell per well in 96-well plates. Pool 5 generated clone sets A and H, Pool 3 generated clone sets B and I, and Pool 2 generated clone sets C and J.

The 96-well plates were imaged, fed with fresh cell culture medium, and subjected to flow cytometry screening of CD52 reporter expression. The steps in this process are described further below. Pre-deposition and within the first seven days after seeding the 96-well plates, the plates were imaged four times on the Cell Metric® CLD (Solentim) at the high resolution (2 m/pixel) setting for clonality documentation. These scans were performed on Day 0 (pre-deposition) and on Day 0 (post-deposition), Day 3, and Day 7. Wells were fed with growth medium, imaged for confluence, and screened by flow cytometry. For the C and J sets from the Pool 2 sort, no clones were observed in the 96-well plates, and therefore there were no clones generated from Pool 2.

Flow cytometry screening of the clones resulted in a Geometric Mean Fluorescence Intensity (GMFI) value for each clone based on its relative CD52 reporter expression. Clones were ranked based on their GMFI values, and the top clones from each set were selected for scale-up. After the preliminary lead candidate clones had been identified, the well images acquired by the Cell Metric® CLD were reviewed to assess monoclonality for the lead clones (as discussed further below).

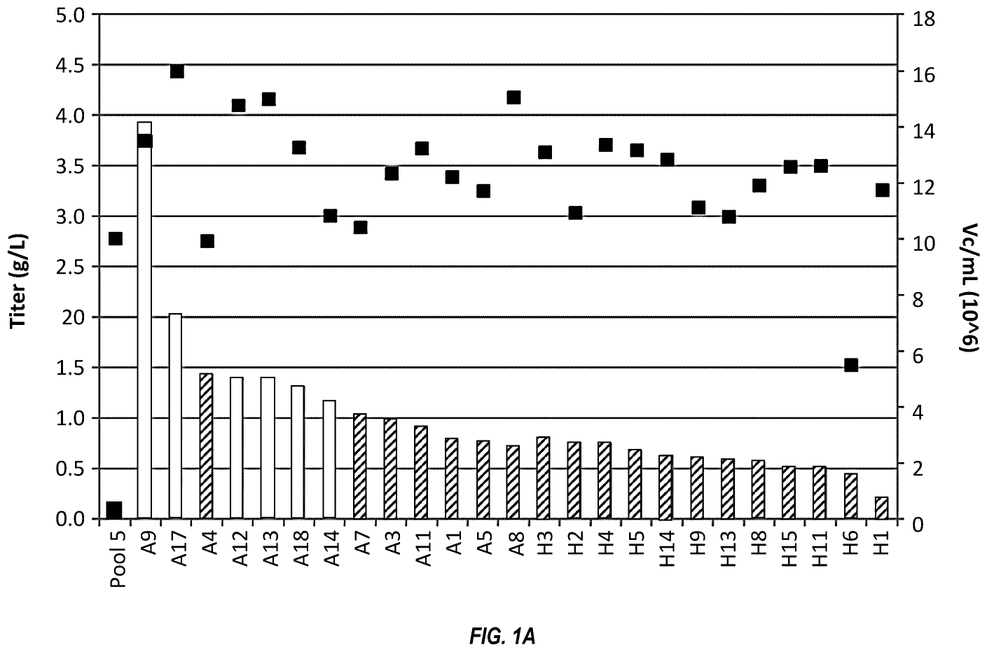

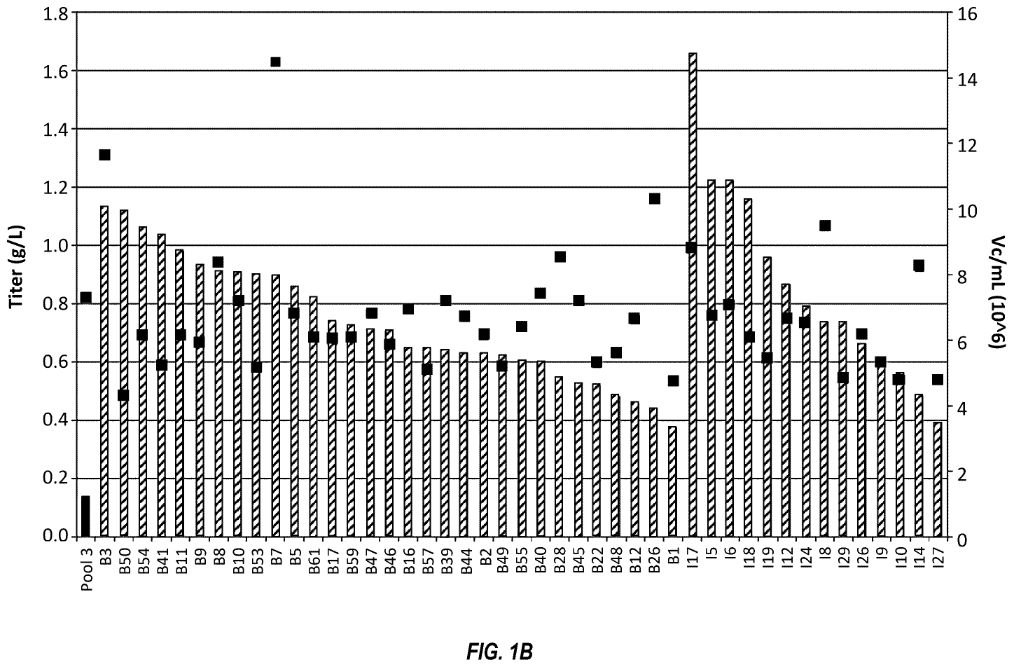

Clones were scaled up, seeded into batch culture, and cryopreserved. All clones completing the scale up process were evaluated in batch culture to determine EBV Ferritin gp220 titer. Each clone was seeded in a ST at a seeding density of 1×10⁶ vc/mL in 10 mL CD CHO/30% Efficient Feed B supplemented with 4 mM L-glutamine (see Table 3 for cell culture conditions). Cell counts and sample harvests were performed on Day 52, and the cultures were terminated. Results are shown in FIGS. 1A-B.

At the time of batch culture set-up, a research cell bank (RCB) was prepared for each clone (4 vials per clone). The cryopreservation medium was CD CHO supplemented with 4 mM L-glutamine and 7% (v/v) DMSO. For each clone, 1×10⁷ cells were cryopreserved per cryovial.

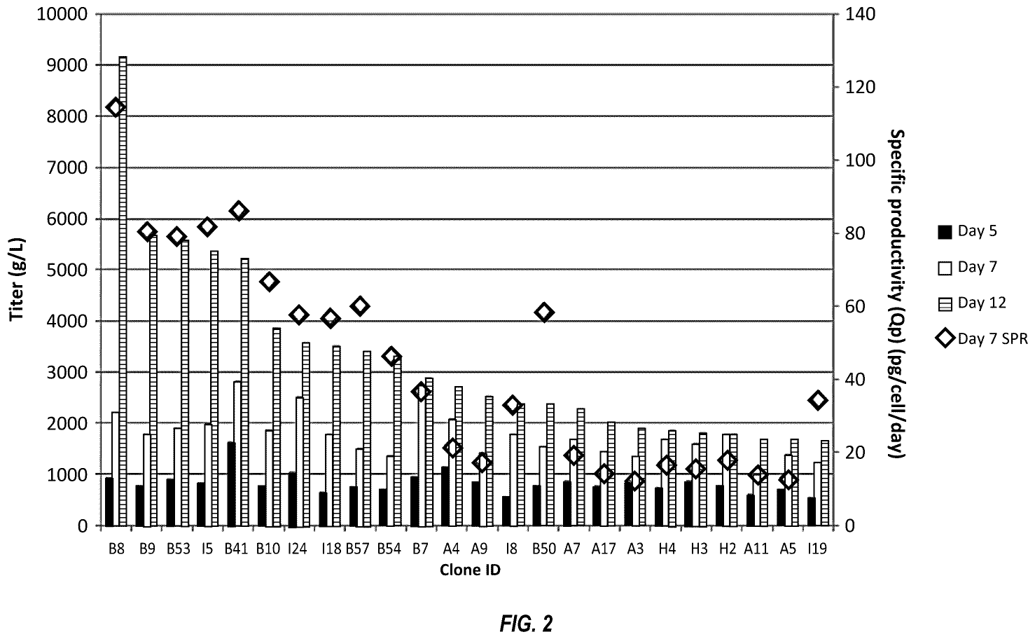

Based on the batch culture results, 24 clones were selected for fed batch screen in the Ambr®15 system. Based on the fed batch results (FIG. 2), six lead candidate clones were selected and one vial of each respective RCB was thawed for pre-MCB preparation. Table 6 lists the six lead candidate clones and the final titers (measured using the Octet method (Octet Red 96, US Breakthrough Lab, Cambridge)) from the fed batch clone screen.

TABLE 6
gp220-ferritin lead candidate clones.

| Clone ID | Day 12 Titer (g/L) |
|---|---|
| B8 | 9.2 |
| B9 | 5.7 |
| B41 | 5.2 |
| B53 | 5.6 |
| I5 | 5.4 |
| I18 | 3.5 |

The Cell Metric® CLD scan images from Days 0 to 7 were utilized for clonality documentation to aid in choosing the lead candidate clones to move forward. Only clones which clearly showed both a single cell on Day 0 of plating and growth emanating only from that single cell as a colony on the subsequent scan days (with no other growth evident in the well) were selected as candidates for further evaluation.

CHO pools expressing EBV Ferritin gp220 (bfpFerr) were generated using an ADC-free CHO cell line development platform. CHO pools were selected for reporter-based flow cytometric sorting to isolate clones, and CHO clones producing EBV Ferritin gp220 were generated. Batch culture evaluation of the clones was used to select 24 clones for a fed batch screen. Based on cell culture performance in the Ambr® fed batch screen, six clones were selected for preparation.

### Example 3: Analysis of C18 gL/gH/gp42 Nanoparticle Binding to Antibodies Confirming Antigen Epitopes

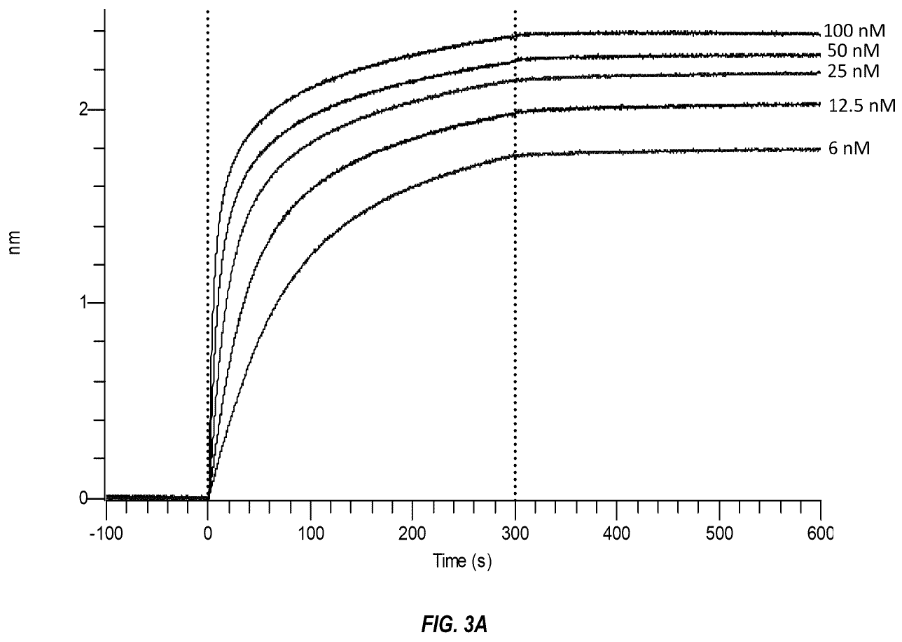

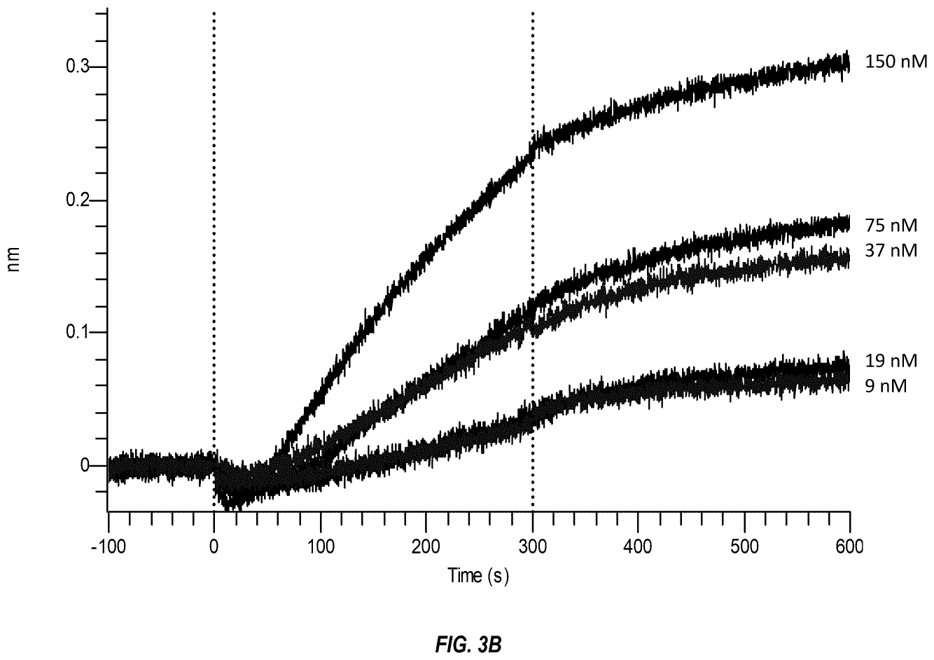

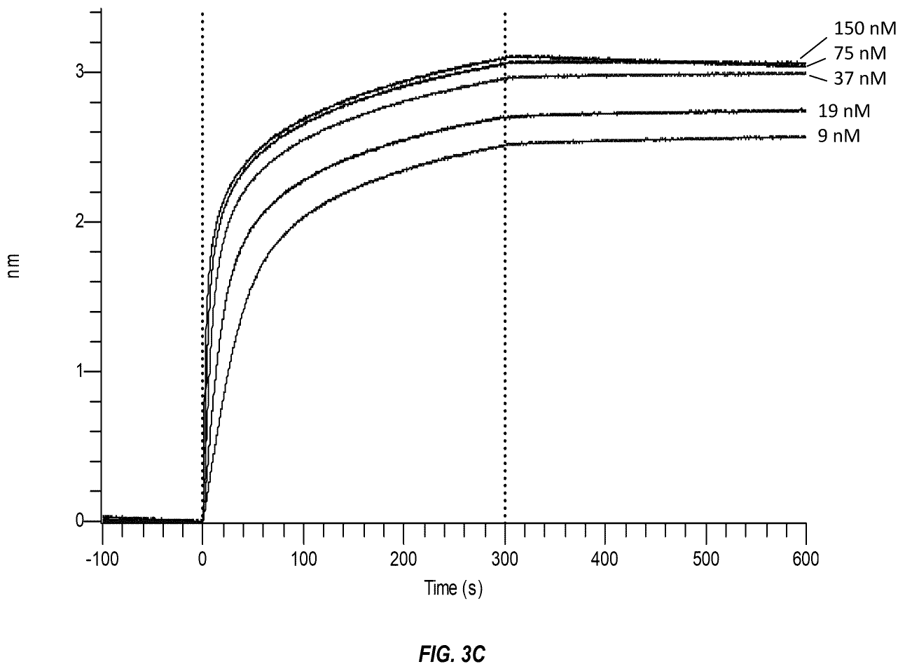

A gL/gH/gp42 NP construct (“C18”, encoding the amino acid sequence of SEQ ID NO: 40) was expressed in 293 expi cells and purified. Binding kinetics of the nanoparticles was assessed using a Sartorius Octet® R8 protein analysis system with either an AMQ anti-mouse FC sensor (Satorius 18-5022) or an AHC2 anti-human FC sensor (Satorius 18-5142). Antibodies (Ammol human mAB anti-gL/gH/gp42 (Creative Biolabs PABC-311), E1D1 mouse mAB anti-gL (Creative Biolabs PABC-524), or 1D8 human mAB anti-gL/gH (purified)) were loaded at 15 μg/ml onto the appropriate sensor. After equilibrating the sensors in kinetic buffer for 12 minutes, a regeneration cycle was run, followed by a baseline step. Antibody was loaded for 300 seconds followed by another baseline for 100 seconds. Antigen association was then measured for 300 seconds followed by a 300 second disassociation in baseline buffer. Reference wells of buffer were used to normalize the readings. FIGS. 3A-C show octet analysis of the C18 gL/gH/gp42 nanoparticle (SEQ ID NO: 11) binding to antibodies confirming antigen epitopes. FIGS. 3A-C show binding curves of the C18 gL/gH/gp42 nanoparticle using Ammol antibody, E1D1 antibody, and 1D8 antibody, respectively. Each antibody binds a different epitope on the C18 antigen. Ammol binds epitopes across gH, gL, and gp42; E1D1 binds an epitope in gL; and 1D8 binds epitopes in gH and gL. Binding curves showed strong interactions of these epitopes at multiple concentrations of antigen, confirming that the antigen structure was intact.

### Example 4: Immunization Against the C18 gL/gH/gp42 Nanoparticle

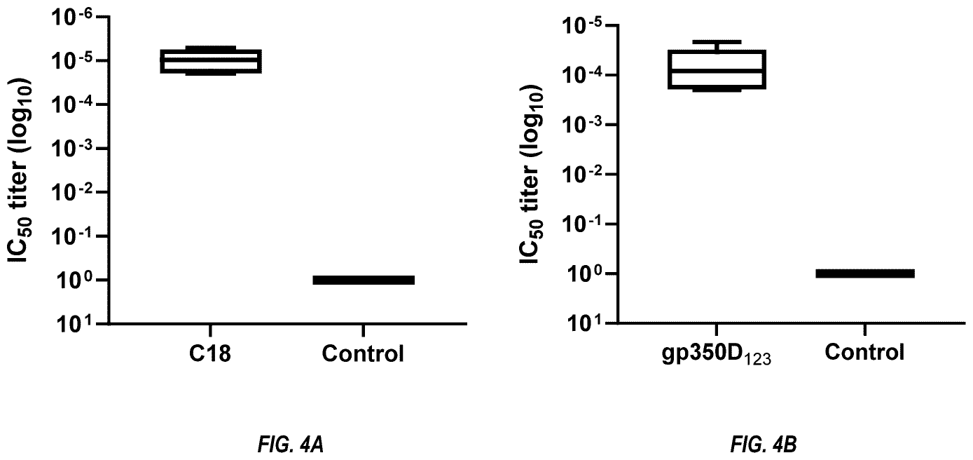

Mice were immunized with compositions comprising the C18 gL/gH/gp42 ferritin nanoparticle (SEQ ID NO: 11) or a gp350D123 ferritin nanoparticle (SEQ ID NO: 14). FIGS. 4A-4B show ELISAs of serum from mice immunized at day 0 and day 21 with 1 μg of either the C18 gL/gH/gp42 ferritin nanoparticle (FIG. 4A, SEQ ID NO: 11) or the gp350D123 ferritin nanoparticle (FIG. 4B, SEQ ID NO: 14). The control was the pre-bleed of the mice before immunization. There were 5 mice in each group. The ELISA plate was coated with 2 μg/ml of His-tagged monomer of the antigens. Mouse serum was diluted 5-fold in series starting at 1/10 dilution and an IC50 was determined from the curve. The mouse serum was detected using an HRP-conjugated Anti-mouse FC antibody (Jackson 115-035-071) at 1:5000 dilution and KPL SureBlue TMB substrate. Absorbance was measured at 650 on a plate reader. Results showed strong immune responses for both nanoparticles at 3 weeks following the second dose.

### Example 5: Electron Microscopy Imaging of the C18 gL/gH/gp42 Nanoparticle

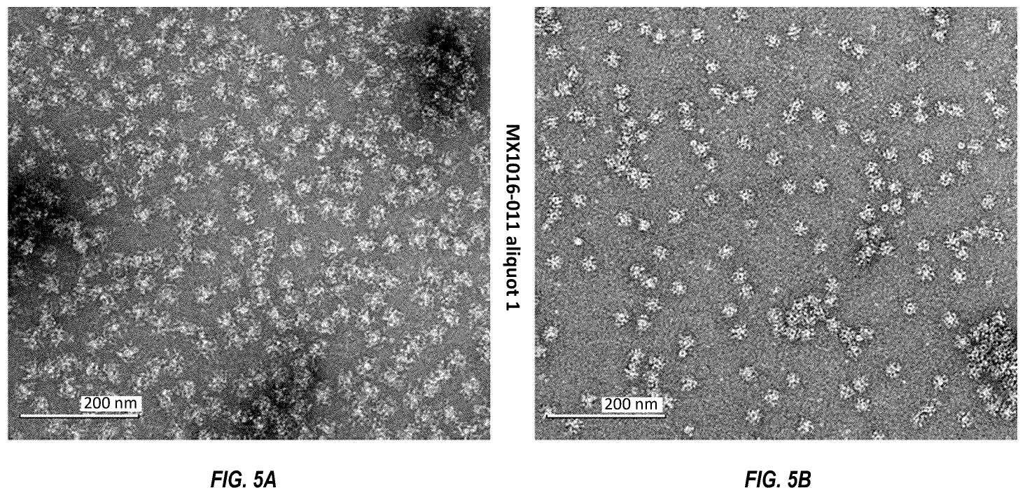

C18 gL/gH/gp42 ferritin nanoparticles (SEQ ID NO: 11) and gp350D123 ferritin nanoparticles (SEQ ID NO: 14) were imaged using electron microscopy negative stain analysis over a layer of continuous carbon supported by nitro-cellulose on a 400-mesh copper grid. The grids were prepared by applying 3 μL of sample suspension to a cleaned grid, blotting with filter paper, and immediately staining with uranyl formate. Electron microscopy was performed using a Thermo Fisher Scientific Glacios Cryo Transmission Electron Microscope (Cryo-TEM) operated at 200 kV and equipped with a Falcon 3 direct electron detector. Grids were clipped into cartridges, transferred into a cassette and then into the Glacios autoloader, all while maintaining the grids at cryogenic temperature (i.e., below −170° C.). Automated data collection was carried out using Leginon software (Suloway et al., Struct Biol, 2005, 151(1):41-60 and Cheng et al., Protein Sci, 2021, 30(1):136-150), where high magnification movies were acquired by selecting targets at a lower magnification. Images of each grid were acquired at multiple scales to assess the overall distribution of the specimen. After identifying potentially suitable target areas for imaging at lower magnifications, high magnification images were acquired at nominal magnifications of 73,000× (0.201 nm/pixel) and 28,000× (0.519 nm/pixel). The images were acquired at a nominal underfocus of −5.5 μm to −3.5 μm and electron doses of ˜10-25 e−/Å2. Samples were prepared and imaged by NIS Nanoimaging services. FIGS. 5A-B show the results of the electron microscopy negative stain analysis of the nanoparticles. FIG. 5A shows the gL/gH/gp42 C18 ferritin nanoparticle and FIG. 5B shows the gp350D123 ferritin nanoparticle. Uniform particle formation can be seen, with few aggregates.

1. A fusion protein comprising:
(a) an Epstein Barr virus (EBV) polypeptide comprising: (i) an EBV gH polypeptide and/or (ii) an EBV gL polypeptide; and
(b) a ferritin comprising the amino acid sequence of SEQ ID NO: 1 with at least one of the following substitutions: (i) a glutamine at a residue corresponding to position N102 of SEQ ID NO: 1; (ii) a glutamine at a residue corresponding to position N145 of SEQ ID NO: 1; or (iii) a combination thereof.

2. The fusion protein of claim 1, wherein the EBV polypeptide further comprises an EBV gp42 polypeptide.

3. (canceled)

4. The fusion protein of claim 1, wherein the ferritin comprises
(a) La glutamine at a residue corresponding to position N102 of SEQ ID NO: 1 and a glutamine at a residue corresponding to position N145 of SEQ ID NO: 1, optionally wherein the ferritin further comprises a glutamine at a residue corresponding to position N18 of SEQ ID NO: 1 and a serine at a residue corresponding to position C30 of SEQ ID NO: 1; and/or
(b) a bullfrog ferritin extension sequence is linked at the N-terminus of the ferritin, wherein the bullfrog ferritin extension sequence comprises the amino acid sequence of SEQ ID NO: 2.

5. (canceled)

6. The fusion protein of claim 2, wherein the EBV polypeptide comprises: (i) an EBV gp42 polypeptide, (ii) an EBV gH polypeptide, and (iii) an EBV gL polypeptide, optionally wherein the EBV gL polypeptide, the EBV gH polypeptide, the EBV gp42 polypeptide, and the ferritin are arranged in N-terminal to C-terminal order.

7. (canceled)

8. The fusion protein of claim 1, further comprising a linker between each EBV polypeptide.

9. The fusion protein of claim 8, wherein
(a) the linker between the EBV gL polypeptide and the EBV gH polypeptide is 44 to 48 amino acids in length, and the linker between the EBV gH polypeptide and the EBV gp42 polypeptide is 32 amino acids in length;
(b) the linker between the EBV gL polypeptide and the EBV gH polypeptide is 46 amino acids in length, and the linker between the EBV gH polypeptide and the EBV gp42 polypeptide is 32 amino acids length;
(c) the linker between the EBV gL polypeptide and the EBV gH polypeptide consists of an amino acid sequence that has at least 80% identity to SEQ ID NO: 3; and/or
(d) the EBV polypeptide further comprises an EBV gp42 polypeptide, and a linker separates the EBV gH polypeptide and the EBV gp42 polypeptide and consists of an amino acid sequence that has at least 80% identity to SEQ ID NO: 4.

10. (canceled)

11. (canceled)

12. The fusion protein of claim 2, further comprising a further linker that separates the ferritin and the EBV gp42 polypeptide.

13. The fusion protein of claim 12, wherein the further linker has a length of 88 amino acids, and/or the further linker consists of an amino acid sequence having at least 80% identity to SEQ ID NO: 5.

14. (canceled)

15. The fusion protein of claim 1, wherein the ferritin comprises the amino acid sequence of SEQ ID NO: 23.

16. The fusion protein of claim 1, wherein
(a) the EBV polypeptide further comprises an EBV gp42 polypeptide, and the EBV gp42 polypeptide comprises an amino acid sequence having at least 80%, 90%, 95%, 96%, 97%, 98%, or 99% identity to residues 856-1038 of any one of SEQ ID NOs: 9-11;
(b) the EBV gH polypeptide comprises an amino acid sequence having at least 80%, 90%, 95%, 96%, 97%, 98%, or 99% identity to residues 162-823 of any one of SEQ ID NOs: 9-11;
optionally wherein the EBV gH polypeptide comprises a leucine at a residue corresponding to position 189 of any one of SEQ ID NOs: 9-11; a glutamic acid at a residue corresponding to position 368 of any one of SEQ ID NOs: 9-11; and a glutamic acid at a residue corresponding to position 429 of any one of SEQ ID NOs: 9-11; and/or
(c) the EBV gL polypeptide comprises an amino acid sequence having at least 80%, at least 90%, at least 95%, at least 96%, at least 97%, at least 98%, or at least 99% identity to residues 1-115 of any one of SEQ ID NOs: 9-11; optionally wherein the EBV gL polypeptide comprises an alanine at a residue corresponding to position 36 of any one of SEQ ID NOs: 9-11; a glutamine at a residue corresponding to position 47 of any one of SEQ ID NOs: 9-11; and a glutamine at a residue corresponding to position 105 of any one of SEQ ID NOs: 9-11.

17. (canceled)

18. (canceled)

19. The fusion protein of claim 1, comprising a sequence having at least 90%, 95%, 96%, 97%, 98%, or 99% identity to (a) amino acid residues 1-1133 of any one of SEQ ID NOs: 9-11, (b) amino acid residues 1-1148 of any one of SEQ ID NOs: 36, 38, or 40, or (c) amino acid residues 1-1156 of any one of SEQ ID NOs: 37, 39, or 41.

20. The fusion protein of claim 1, comprising a mammalian leader sequence positioned at the N-terminus of the fusion protein, optionally wherein the fusion protein comprises an EBV gL polypeptide, an EBV gH polypeptide, an EBV gp42 polypeptide, and a ferritin, wherein the EBV gL polypeptide, the EBV gH polypeptide, the EBV gp42 polypeptide, and the ferritin are arranged in N-terminal to C-terminal order within the EBV polypeptide, and the mammalian leader sequence is positioned N-terminal to the EBV gL polypeptide.

21. The fusion protein of claim 20, wherein the mammalian leader sequence is selected from SEQ ID NO: 12 and SEQ ID NO: 13.

22. A fusion protein comprising:
(a) an Epstein Barr virus (EBV) polypeptide comprising an EBV gL polypeptide and an EBV gH polypeptide, wherein the EBV gL polypeptide comprises the sequence of residues 1-115 of SEQ ID NO: 11 and the EBV gH polypeptide comprises the sequence of residues 162-823 of SEQ ID NO: 11; optionally wherein a linker separates the EBV gL polypeptide and the EBV gH polypeptide and comprises the amino acid sequence of SEQ ID NO: 3;
(b) a ferritin polypeptide comprising the amino acid sequence of SEQ ID NO: 1, or a ferritin polypeptide comprising the amino acid sequence of SEQ ID NO: 1 and at least one mutation selected from one or more of a glutamine at a residue corresponding to position N18 of SEQ ID NO: 1; a serine at a residue corresponding to position C30 of SEQ ID NO: 1; a glutamine at a residue corresponding to position N102 of SEQ ID NO: 1; and a glutamine at a residue corresponding to position N145 of SEQ ID NO: 1; and
(c) a bullfrog ferritin extension sequence that is linked at the N-terminus of the ferritin, wherein the bullfrog ferritin extension sequence comprises the amino acid sequence of SEQ ID NO: 2, and further optionally a mammalian leader sequence positioned at the N-terminus of the fusion protein, wherein the mammalian leader sequence comprises the amino acid sequence of SEQ ID NO: 12 or SEQ ID NO: 13;
wherein the EBV gL polypeptide, the EBV gH polypeptide, and the ferritin are arranged in N-terminal to C-terminal order within the EBV polypeptide, and the optional mammalian leader sequence is positioned N-terminal to the EBV gL polypeptide.

23. The fusion protein of claim 1, wherein:
(a) the EBV polypeptide further comprises an EBV gp42 polypeptide, and wherein the EBV gL polypeptide comprises the amino acid sequence of residues 1-115 of SEQ ID NO: 11, the EBV gH polypeptide comprises the amino acid sequence of residues 162-823 of SEQ ID NO: 11, and the EBV gp42 polypeptide comprises the amino acid sequence of residues 856-1038 of SEQ ID NO: 11;
(b) the ferritin comprises or consists of the amino acid sequence of SEQ ID NO: 24; and
(c) optionally, the fusion protein comprises a mammalian leader sequence positioned at the N-terminus of the fusion protein, wherein the mammalian leader sequence comprises the amino acid sequence of SEQ ID NO: 12 or SEQ ID NO: 13;
wherein the EBV gL polypeptide, the EBV gH polypeptide, the EBV gp42 polypeptide and the ferritin are arranged in N-terminal to C-terminal order within the EBV polypeptide, and the optional mammalian leader sequence is positioned N-terminal to the EBV gL polypeptide.

24.-27. (canceled)

28. A fusion protein comprising or consisting of the amino acid sequence of any one of SEQ ID NOs: 9-11 or 36-41.

29. (canceled)

30. A fusion protein comprising (i) an EBV gp220 polypeptide that comprises an amino acid sequence having at least 80%, at least 85%, at least 90%, at least 95%, at least 96%, at least 97%, at least 98%, at least 99%, or 100% sequence identity to residues 1-425 of SEQ ID NO: 14, and (ii) a ferritin comprising the amino acid sequence of any one of SEQ ID NOs: 23, 24, 42, and 43; optionally wherein the amino acid sequence of the fusion protein consists of SEQ ID NO: 25 or 35.

31. (canceled)

32. A ferritin nanoparticle comprising the fusion protein of claim 1.

33. A composition or kit comprising a first ferritin nanoparticle of claim 32, and optionally a second ferritin nanoparticle comprising a second fusion protein comprising an EBV gp220 polypeptide and a ferritin.

34. The composition or kit of claim 33, wherein
(a) the EBV gp220 polypeptide comprises an amino acid sequence having at least 80%, at least 85%, at least 90%, at least 95%, at least 96%, at least 97%, at least 98%, at least 99%, or 100% sequence identity to residues 1-425 of SEQ ID NO: 14;
(b) the ferritin in the second fusion protein comprises a serine or a cysteine at a residue corresponding to position 110 of SEQ ID NO: 1; optionally wherein the ferritin comprises an amino acid sequence having at least 80% identity to residues 435-600 of SEQ ID NO:14;
(c) the second fusion protein comprises an amino acid sequence having at least 80%, at least 85%, at least 90%, at least 95%, at least 96%, at least 97%, at least 98%, at least 99%, or 100% sequence identity to SEQ ID NO: 14, optionally wherein position C544 of SEQ ID NO: 14 is substituted to a serine;
(d) the first ferritin nanoparticle comprises a fusion protein consisting of the amino acid sequence of any one of SEQ ID NOs: 9-11, and (a) the second ferritin nanoparticle comprises a second fusion protein consisting of the amino acid sequence of SEQ ID NO: 14, or (b) the second ferritin nanoparticle comprises a second fusion protein consisting of the amino acid sequence of SEQ ID NO: 14 with a substitution to a serine at position C544 of SEQ ID NO: 14; and/or
(e) the first ferritin nanoparticle comprises a fusion protein consisting of the amino acid sequence of SEQ ID NO: 11 and the second ferritin nanoparticle comprises a second fusion protein consisting of the amino acid sequence of SEQ ID NO: 14.

35.-38. (canceled)

39. A pharmaceutical composition comprising the fusion protein of claim 1; and a pharmaceutically acceptable carrier.

40. (canceled)

41. A bivalent vaccine comprising the composition of claim 33, wherein the fusion protein and the second fusion protein are present in a 1:1 ratio by weight or wherein the fusion protein and the second fusion protein are present in a 1:1 molar ratio.

42. (canceled)

43. A method of eliciting an immune response to EBV in a subject, protecting a subject against infection with EBV, or preventing an EBV-associated disease in a subject, comprising administering to the subject the fusion protein of claim 1.

44. A nucleic acid encoding the fusion protein of claim 1.

45. An expression vector comprising the nucleic acid of claim 44.

46. A host cell comprising the nucleic acid of claim 44.

47. A method of preparing the fusion protein according to claim 1, comprising: culturing a host cell under conditions whereby the fusion protein is expressed in the host cell, and recovering the fusion protein from the cultured host cell culture.

48.-53. (canceled)

54. A ferritin protein consisting of the amino acid sequence of SEQ ID NO: 24.
